(19) United States
(12) Patent Application Publication
(10) Pub. No.: US 2026/0049308 A1
(43) Pub. Date: Feb. 19, 2026

(54) COMPOSITIONS FOR AND METHODS OF EDITING THE GENOME

(71) Applicant: Duke University, Durham, NC (US)

(72) Inventors: Dwight Koeberl, Durham, NC (US); Benjamin Arnson, Durham, NC (US); Bryan Cullen, Durham, NC (US)

(21) Appl. No.: 19/185,073

(22) Filed: Apr. 21, 2025

Related U.S. Application Data
(63) Continuation of application No. 18/552,693.
(60) Provisional application No. 63/169,287, filed on Apr. 1, 2021.

(51) Int. Cl. C12N 15/11; A61K 48/00; A61P 3/00; C12N 9/22; C12N 15/86; C12N 15/90

(52) U.S. Cl. CPC C12N 15/111; A61K 48/005; A61P 3/00; C12N 9/22; C12N 15/86; C12N 15/907; C12N 2310/20; C12N 2750/14143; C12N 2830/008

(57) ABSTRACT

Disclosed herein are compositions for and methods of editing in vivo a defective gene such as GAA.

### CROSS-REFERENCE TO RELATED APPLICATIONS

This application is a is a § 371 U.S. National Stage of International Application PCT/US2022/-23056, filed 1 Apr. 2022 which claims priority to U.S. Provisional Application No. 63/169,287 filed 1 Apr. 2021, which are incorporated herein in their entireties.

### STATEMENT REGARDING FEDERAL FUNDING

This invention was made with Government support under Federal Grant No. R21AR073470 awarded by the National Institute of Arthritis and Musculoskeletal and Skin Diseases (NIH/NIAMS), R01DK105434-04 awarded by the National Institute of Diabetes and Digestive and Kidney Disease (NIH/NIDDK), and R01AR065873 awarded by the National Institute of Arthritis and Musculoskeletal and Skin Diseases (NIH/NIDDK). The Federal Government has certain rights to this invention.

### REFERENCE TO THE SEQUENCE LISTING

The application contains a Sequence Listing XML which has been submitted in XML format via EFS-Web and is hereby incorporated by reference in its entirety. The XML copy, created on 10 Nov. 2025 is named 210-132-CIP-SEQ.xml and is 217,130 bytes in size.

### BACKGROUND

Early treatment is needed to reverse the symptoms of Pompe disease (glycogen storage disease type II), a lysosomal storage disorder caused by acid α-glucosidase deficiency that leads to the accumulation of lysosomal glycogen and debilitating weakness. Patients with Pompe disease receive life-prolonging therapy with enzyme replacement therapy (ERT) consisting of recombinant human (rh) acid α-glucosidase (GAA); however, even treatment in the first days of life sometimes fails to prevent the muscle weakness of infantile-onset Pompe disease (IOPD) patients. Recently gene replacement therapy with adeno-associated virus (AAV) vectors has entered early-stage clinical trials in adults with Pompe disease. However, the youngest patients with IOPD will not be enrolled in clinical trials with gene replacement therapy, because efficacy will diminish due to the loss of episomal vector genomes from the growing liver. Moreover, the re-administration of AAV vectors is currently ineffective due to the formation of anti-AAV antibodies.

Consequently, there is a critical unmet need for the development of improved genetic therapies to stably treat Pompe disease and other disorders of metabolism early in life. In absence of an effective genetic therapy for infants with IOPD, this population will continue to experience progressive loss of muscle function. Consequently, the present disclosure provides compositions for and methods of repairing a defective GAA gene and treating a subject having Pompe disease, which can be used alone or in combination with other treatments.

### BRIEF DESCRIPTION OF THE FIGURES

FIG. 1A-FIG. 1F show the schematic of genome editing.

FIG. 1A shows two editing vectors, containing a EF1α promoter (Sanjana N E, et al. (2014) Nat Methods. 11:783-784) to drive Staph aureus Cas9 (SaCas9) expression and a U6 promoter to drive sgRNA expression (Mefferd A L, et al. (2015) RNA. 21:1683-1689). Alternatively, a glutamine tRNA gene promoter can drive sgRNA expression (Mefferd et al. 2015). Here, the repair template for IVS1 is 650 bp and 620 bp for ΔT525.

FIG. 1B shows a schematic of HDR at the double-stranded DNA break created to insert an EF1αpromoter (top) or a minimal G6PC promoter (bottom) to drive GAA expression from the ATG (start codon) of the GAA gene.

FIG. 1C shows successful editing at the IVS1 variant following transfection of patient fibroblasts, which also introduced an MfeI restriction site without changing the amino acid sequence. MfeI digestion revealed >52% modification through HDR. Transfection with a scrambled sgRNA revealed no cutting (last lane).

FIG. 1D shows that editing of the ΔT525 variant in transfected patient fibroblasts detected by HDR through cutting an introduced BamHI site and revealed >35% HDR. Transfection with a scrambled sgRNA revealed no cutting (last lane).

FIG. 1E shows IVS1 editing with rAAV9-CRISPR in cultured human cardiomyocytes and revealed up to 7.2% HDR following transduction with the rAAV9 vector. rAAV9-GFP (control) transduced cardiomyocytes had 0% HDR detected (last lane).

FIG. 1F shows western blotting that revealed GAA expression in patient fibroblasts edited to repair the ΔT525 variant by delivering both the sgRNA and SaCas9 (first 3 lanes).

FIG. 2A-FIG. 2C shows the editing the 1826DupA variant.

FIG. 2A shows that a G6PC minimal promoter driving human GAA in an AAV9 vector produced high GAA activity in the heart (left) and quadriceps (right) of GAA knockout mice, which was significantly higher than wildtype GAA activity.

FIG. 2B shows a single vector construct containing a CRISPR/Cas9 for editing the 1826DupA variant with the G6PC minimal promoter to drive SaCas9 expression.

FIG. 2C shows that the transfection of fibroblasts to demonstrate the integration of the repair template flanking the 1826DupA variant. The repair template introduced a HgaI restriction enzyme recognition site, and only cells that had undergone HDR to integrate the repair template would generate smaller DNA fragments upon HgaI restriction digestion (arrows). No HDR mediated integration of the repair template was observed for vectors containing the CRISPR/Cas9 that used either the CB promoter or CMV promoter instead of the G6PC promoter.

FIG. 3A-FIG. 3E show an editing strategy to create a liver depot for GAA production.

FIG. 3A show the donor vector contains homology from intron 1/exon 2 to drive HDR and integration of the transgene upstream from the GAA gene's start codon. The polyadenylation signal prevents transcription of mutant GAA from the GAA gene promoter. In both vectors, the promoter used to drive SaCas9 expression or GAA expression was either the CB (chicken beta-actin promoter/cytomegalovirus enhancer) previously demonstrated to drive high level GAA expression in muscle (Sun B, et al. (2005) Mol Ther. 11:57-65) or the LP1 (liver-specific promoter) that drives high level GAA expression in liver accompanied by cross-correction of muscle GAA deficiency (Franco L M, et al. (2005) Mol Ther. 12:876-884; Nathwani A C, et al. (2007) Blood. 109:1414-1421). FIG. 3B shows the second rAAV9 vector delivered CRISPR/Cas9 to cleave the PAM in intron 1, leading to transgene integration (FIG. 3C) and stable, high level expression of GAA (FIG. 3D and FIG. 3E). Specifically, FIG. 3C shows that the incorporation of the repair template occurred only when delivered with the Cas9/gRNA vector. When the cells were passaged, the repair template was still detectable in the genome. FIG. 3D shows GAA protein expression in the transfected cells. Here, cells transfected with the CB-GAA vector only showed a greater decrease in GAA protein levels when the cells were passaged. Cells transfected with both vectors, however, maintained GAA expression better when they were passaged. Here, insertion of the transgene stably corrects GAA deficiency throughout the tissues, due to the secretion of GAA from the liver and the mannose-6-phosphate receptor mediated uptake of GAA from blood in the other tissues. The LP1 promoter expresses GAA specifically in the liver to induce immune tolerance to GAA and prevent anti-GAA antibody formation, thereby increasing the efficacy of treatment in Pompe disease.

FIG. 4 is a map of the construct titled “delT525 623 bp-Repair U6-sgRNA CMV-SaCas9-HA-synPolyA”.

FIG. 5 is a map of the construct titled “delT525 400 bp-Repair GlntRNA-sgRNA CB-SaCas9-HA-synPolyA”.

FIG. 6 is a map of the construct titled “delT525 400 bp-Repair GlntRNA-sgRNA G6Pcmin303-SaCas9-HA-synPolyA”.

FIG. 7 is a map of the construct titled “IVS1 212 bp-RepairArms GlntRNA-sgRNA CMV-SaCas9-HA-synPolyA”.

FIG. 8 is a map of the construct titled “IVS1 210 bp-RepairArms GlntRNA-sgRNA EF1a-SaCas9-HA-synPolyA”.

FIG. 9 is a map of the construct titled “m1826DupA 404 bp-Repair GlntRNA-sgRNA1 CMV-SaCas9-HA-synPolyA”.

FIG. 10 is a map of the construct titled “m1826DupA 404 bp-Repair GlntRNA-sgRNA2 CMV-SaCas9-HA-synPolyA”.

FIG. 11 is a map of the construct titled “mIVS1 GlntRNA-sgRNA1 LP1-SaCas9-HA-synPolyA”.

FIG. 12 is a map of the construct titled “mIVS1 212 bp-RepairArms LP1-hGAA-hGHPolyA”.

FIG. 13 is a map of the construct titled “delT525 623 bp-Repair GlntRNA-sgRNA EF1a-SaCas9-HA-synPolyA”.

FIG. 14 is a map of the construct titled “IVS1 212 bp-RepairArms GlntRNA-sgRNA CB-SaCas9-HA-synPolyA”.

FIG. 15 is a map of the construct titled “IVS1 212 bp-RepairArms GlntRNA-sgRNA LP1-SaCas9-HA-synPolyA”.

FIG. 16 is a map of the construct titled “IVS1 212 bp-RepairArms GlntRNA-sgRNA G6Pcmin303-SaCas9-HA-synPolyA”.

FIG. 17 is a map of the construct titled “IVS1 212 bp-RepairArms-StopCodonsynPolyA GlntRNA-sgRNA G6Pcmin303-SaCas9-HA-synPolyA”.

FIG. 18 is a map of the construct titled “IVS1 LP1-SaCas9 human sgRNA”.

FIG. 19 is a map of the construct titled “IVS1 LP1-hGAA-hGH human homolgy arms”.

FIG. 20 is a map of the construct titled “mouse DupA G6Pc303 guide 1”.

FIG. 21 is a map of the construct titled “mouse DupA G6Pc303 guide 2”.

### BRIEF SUMMARY

Disclosed herein is an isolated nucleic acid molecule, comprising: a nucleic acid sequence encoding a repair template for Pompe disease.

Disclosed herein is an isolated nucleic acid molecule for repairing the IVS1 variant of Pompe disease, comprising a nucleic acid sequence encoding a repair template, wherein the repair template comprises a first sequence having homology to a sequence in intron 1 of GAA and a second sequence having homology to a sequence in exon 2 of GAA, wherein the homologous sequences flank a promoter; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is an isolated nucleic acid molecule for repairing the ΔT525 variant of Pompe disease, comprising a nucleic acid sequence encoding a repair template, wherein the repair template comprises a sequence having homology to a sequence in exon 2 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is an isolated nucleic acid molecule for repairing the 1826DupA variant of Pompe disease, comprising a nucleic acid sequence encoding a repair template, wherein the repair template comprises a sequence having homology to a sequence in exon 13 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is an isolated donor nucleic acid molecule, comprising a nucleic acid sequence encoding a repair template, a promoter operably linked to the repair template, and a poly A sequence.

Disclosed herein is an isolated CRISPR nucleic acid molecule comprising a nucleic acid sequence encoding a guide RNA (gRNA) operably linked to a first promoter and a nucleic acid sequence encoding a Cas9 nuclease operably linked to second promoter.

Disclosed herein is an isolated nucleic acid molecule for repairing the IVS1 variant of Pompe disease, comprising a nucleic acid sequence encoding a repair template, wherein the repair template comprises a first sequence having homology to a sequence in intron 1 of GAA and a second sequence having homology to a sequence in exon 2 of GAA, wherein the homologous sequences flank a promoter; a nucleic acid sequence for a promoter that is operably linked to a nucleic acid sequence encoding a guide RNA (gRNA); and a nucleic acid sequence encoding a SaCas9 nuclease and a promoter operably linked to the SaCas9.

Disclosed herein is an isolated nucleic acid molecule for repairing the ΔT525 variant of Pompe disease, comprising a nucleic acid sequence encoding a repair template, wherein the repair template comprises a sequence having homology to a sequence in exon 2 of GAA; a nucleic acid sequence for a promoter that is operably linked to a nucleic acid sequence encoding a guide RNA (gRNA); and a nucleic acid sequence encoding a SaCas9 nuclease and a promoter operably linked to the SaCas9.

Disclosed herein is an isolated nucleic acid molecule for repairing the 1826DupA variant of Pompe disease, comprising a nucleic acid sequence encoding a repair template, wherein the repair template comprises a sequence having homology to a sequence in exon 13 of GAA; a nucleic acid sequence for a promoter that is operably linked to a nucleic acid sequence encoding a guide RNA (gRNA); and a nucleic acid sequence encoding a SaCas9 nuclease and a promoter operably linked to the SaCas9.

Disclosed herein is an isolated donor nucleic acid molecule, comprising a nucleic acid sequence encoding a repair template, a promoter operably linked to the repair template, and a poly A sequence.

Disclosed herein is an isolated CRISPR nucleic acid molecule comprising a nucleic acid sequence encoding a guide RNA (gRNA) operably linked to a first promoter and a nucleic acid sequence encoding a SaCas9 nuclease operably linked to second promoter.

Disclosed herein is a gene editing system for stably integrating an alpha-glucosidase transgene into one or more cells, comprising a first vector comprising an isolated donor nucleic acid molecule; and a second vector comprising an isolated CRISPR nucleic acid molecule.

Disclosed herein is a vector comprising a disclosed isolated nucleic acid molecule. Disclosed herein is a vector comprising a disclosed isolated donor nucleic acid molecule. Disclosed herein is a vector comprising a disclosed isolated CRISPR nucleic acid molecule.

Disclosed herein is a method of repairing a defective GAA gene, the method comprising contacting cells with a disclosed isolated nucleic acid molecule, wherein, following expression of the nucleic acid molecule, the defective GAA gene is repaired in the cells.

Disclosed herein is a method of repairing a defective GAA gene, the method comprising contacting cells with a disclosed isolated donor nucleic acid molecule; and contacting the cells with the disclosed isolated CRISPR nucleic acid molecule; wherein, following expression of the nucleic acid molecules, the defective GAA gene is repaired in the cells.

Disclosed herein is a method of repairing a defective GAA gene, the method comprising administering to a subject a therapeutically effective amount of a disclosed vector comprising a disclosed isolated nucleic acid molecule, wherein, following expression of the nucleic acid molecule, the defective GAA gene is repaired in cells of the subject.

Disclosed herein is a method of repairing a defective GAA gene, the method comprising administering to a subject a therapeutically effective amount of a disclosed vector comprising a disclosed isolated donor nucleic acid molecule; and administering to the subject a disclosed vector comprising a disclosed isolated CRISPR nucleic acid molecule; wherein, following expression of the nucleic acid molecules, the defective GAA gene is repaired in cells of the subject.

Disclosed herein is a method of repairing a defective GAA gene, the method comprising administering to a subject a therapeutically effective amount of a disclosed vector comprising a disclosed isolated nucleic acid molecule for any mutation or any variant leading to Pompe disease, comprising a nucleic acid sequence encoding a repair template, wherein the repair template comprises a sequence having homology to a wild-type sequence of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9; wherein, following expression of the nucleic acid molecules, the defective GAA gene is repaired in cells of the subject.

Disclosed herein is a method of repairing a defective GAA gene, the method comprising administering to a subject a therapeutically effective amount of a disclosed vector comprising a disclosed isolated nucleic acid molecule for any mutation or any variant leading to Pompe disease, comprising a nucleic acid sequence encoding a repair template, wherein the repair template comprises a sequence having homology to a wild-type sequence of GAA; a nucleic acid sequence for a promoter that is operably linked to a nucleic acid sequence encoding a guide RNA (gRNA); and a nucleic acid sequence encoding a SaCas9 nuclease and a promoter operably linked to the SaCas9; wherein, following expression of the nucleic acid molecules, the defective GAA gene is repaired in cells of the subject.

Disclosed herein is a method of repairing a defective GAA gene, the method comprising administering to a subject a disclosed gene editing system, wherein, following expression of the nucleic acid molecules, the defective GAA gene is repaired in cells of the subject.

Disclosed herein is an in vivo method for treating a subject having Pompe disease, the method comprising administering to the subject a therapeutically effective amount of a disclosed vector comprising the isolated donor nucleic acid molecule; and administering to the subject therapeutically effective amount of a disclosed vector comprising the isolated CRISPR nucleic acid molecule; wherein the Cas9 creates a double-strand break (DSB) on both sides of a mutation in the GAA locus that results in a permanent integration of the repair template, thereby repairing the defect underlying Pompe disease.

Disclosed herein is an in vivo method for treating a subject having Pompe disease, the method comprising administering to the subject a therapeutically effective amount of a disclosed vector comprising the isolated donor nucleic acid molecule; and administering to the subject therapeutically effective amount of a disclosed vector comprising the isolated CRISPR nucleic acid molecule; wherein the SaCas9 creates a double-strand break (DSB) on both sides of a mutation in the GAA locus that results in a permanent integration of the repair template, thereby repairing the defect underlying Pompe disease.

Disclosed herein is an in vivo method for treating a subject having Pompe disease, the method comprising administering to the subject a therapeutically effective amount of a vector comprising an isolated donor nucleic acid molecule, comprising a nucleic acid sequence encoding a repair template, a promoter operably linked to the repair template, and a poly A sequence; and administering to the subject therapeutically effective amount of a vector comprising an isolated CRISPR nucleic acid molecule comprising a nucleic acid sequence encoding a guide RNA (gRNA) operably linked to a first promoter and a nucleic acid sequence encoding a Cas9 nuclease operably linked to second promoter; wherein the Cas9 creates a double-strand break (DSB) on both sides of a mutation in the GAA locus that results in a permanent integration of the repair template, thereby repairing the defect underlying Pompe disease.

Disclosed herein is an in vivo method for treating a subject having Pompe disease, the method comprising administering to the subject a therapeutically effective amount of a vector comprising an isolated donor nucleic acid molecule, comprising a nucleic acid sequence encoding a repair template, a promoter operably linked to the repair template, and a poly A sequence; and administering to the subject therapeutically effective amount of a vector comprising an isolated CRISPR nucleic acid molecule comprising a nucleic acid sequence encoding a guide RNA (gRNA) operably linked to a first promoter and a nucleic acid sequence encoding a SaCas9 nuclease operably linked to second promoter; wherein the SaCas9 creates a double-strand break (DSB) on both sides of a mutation in the GAA locus that results in a permanent integration of the repair template, thereby repairing the defect underlying Pompe disease.

Disclosed herein is a method of validating the efficacy of a gene editing system, the method comprising contacting cells with a disclosed isolated nucleic acid molecule; measuring the expression of GAA in the edited cells; and comparing the resulting GAA expression level in the edited cells to the GAA expression level in control cells, wherein the gene editing system is effective when the GAA expression in the edited cells is greater than the GAA expression level in control cells.

Disclosed herein is a method of validating the efficacy of a gene editing system, the method comprising contacting cells with a disclosed isolated nucleic acid molecule; measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

Disclosed herein is a method of validating the efficacy of a gene editing system in a subject, the method comprising administering to the subject a disclosed vector comprising a disclosed isolated nucleic acid molecule; obtaining a biological sample of cells targets for editing; measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

Disclosed herein is a method of validating the efficacy of a gene editing system in a subject, the method comprising administering to the subject a disclosed vector comprising a disclosed isolated nucleic acid molecule; obtaining a biological sample of cells targets for editing; measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

### DETAILED DESCRIPTION

The present disclosure describes formulations, compounded compositions, kits, capsules, containers, and/or methods thereof. It is to be understood that the inventive aspects of which are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention.

### A. Definitions

Before the present compounds, compositions, articles, systems, devices, and/or methods are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

This disclosure describes inventive concepts with reference to specific examples. However, the intent is to cover all modifications, equivalents, and alternatives of the inventive concepts that are consistent with this disclosure.

As used in the specification and the appended claims, the singular forms “a”, “an”, and “the” include plural referents unless the context clearly dictates otherwise.

The phrase “consisting essentially of” limits the scope of a claim to the recited components in a composition or the recited steps in a method as well as those that do not materially affect the basic and novel characteristic or characteristics of the claimed composition or claimed method. The phrase “consisting of” excludes any component, step, or element that is not recited in the claim. The phrase “comprising” is synonymous with “including”, “containing”, or “characterized by”, and is inclusive or open-ended. “Comprising” does not exclude additional, unrecited components or steps.

As used herein, when referring to any numerical value, the term “about” means a value falling within a range that is ±10% of the stated value.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

References in the specification and concluding claims to parts by weight of a particular element or component in a composition denotes the weight relationship between the element or component and any other elements or components in the composition or article for which a part by weight is expressed. Thus, in a compound containing 2 parts by weight component X and 5 parts by weight component Y, X and Y are present at a weight ratio of 2:5, and are present in such ratio regardless of whether additional components are contained in the compound.

As used herein, the terms “optional” or “optionally” means that the subsequently described event or circumstance can or cannot occur, and that the description includes instances where said event or circumstance occurs and instances where it does not. In an aspect, a disclosed method can optionally comprise one or more additional steps, such as, for example, repeating an administering step or altering an administering step.

As used herein, “isolated” refers to a nucleic acid molecule or a nucleic acid sequence that has been substantially separated, produced apart from, or purified away from other biological components in the cell or tissue of an organism in which the component occurs, such as other cells, chromosomal and extrachromosomal DNA and RNA, and proteins. Nucleic acids and proteins that have been “isolated” include nucleic acids and proteins purified by standard purification methods. The term also embraces nucleic acids and proteins prepared by recombinant expression in a host cell as well as chemically synthesized nucleic acids and proteins. Isolated proteins or nucleic acids, or cells containing such, in some examples are at least 50% pure, such as at least 75%, at least 80%, at least 90%, at least 95%, at least 98%, or at least 100% pure.

As used herein, the term “subject” refers to the target of administration, e.g., a human being. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). Thus, the subject of the herein disclosed methods can be a vertebrate, such as a mammal, a fish, a bird, a reptile, or an amphibian. Alternatively, the subject of the herein disclosed methods can be a human, non-human primate, horse, pig, rabbit, dog, sheep, goat, cow, cat, guinea pig, or rodent. The term does not denote a particular age or sex, and thus, adult and child subjects, as well as fetuses, whether male or female, are intended to be covered. In an aspect, a subject can be a human patient. In an aspect, a subject can have a disease or disorder, be suspected of having a disease or disorder, or be at risk of developing a disease or disorder (e.g., Pompe disease or a genetic disease or disorder).

As used herein, a “regulatory element” can refer to promoters, enhancers, internal ribosomal entry sites (IRES), and other expression control elements (e.g., transcription termination signals, such as polyadenylation signals and poly-U sequences). Regulatory elements can include those that direct constitutive expression of a nucleotide sequence in many types of host cells and those that direct expression of the nucleotide sequence only in certain host cells (e.g., tissue-specific regulatory sequences).

As used herein, the term “diagnosed” means having been subjected to an examination by a person of skill, for example, a physician, and found to have a condition that can be diagnosed or treated by one or more of the disclosed isolated nucleic acid molecules, disclosed vectors, disclosed pharmaceutical formulations, or a combination thereof, or by one or more of the disclosed methods. For example, “diagnosed with a disease or disorder” means having been subjected to an examination by a person of skill, for example, a physician, and found to have a condition (e.g., Pompe disease or a genetic disease or disorder) that can be treated by one or more of the disclosed isolated nucleic acid molecules, disclosed vectors, disclosed pharmaceutical formulations, or a combination thereof, or by one or more of the disclosed methods. For example, “suspected of having a disease or disorder” can mean having been subjected to an examination by a person of skill, for example, a physician, and found to have a condition (e.g., Pompe disease or a genetic disease or disorder) that can likely be treated by one or more of by one or more of the disclosed isolated nucleic acid molecules, disclosed vectors, disclosed pharmaceutical formulations, or a combination thereof, or by one or more of the disclosed methods. In an aspect, an examination can be physical, can involve various tests (e.g., blood tests, genotyping, biopsies, etc.) and assays (e.g., enzymatic assay), or a combination thereof.

A “patient” refers to a subject afflicted with a disease or disorder (e.g., Pompe disease or a genetic disease or disorder). In an aspect, a patient can refer to a subject that has been diagnosed with or is suspected of having a disease or disorder. In an aspect, a patient can refer to a subject that has been diagnosed with or is suspected of having a disease or disorder and is seeking treatment or receiving treatment for a disease or disorder.

As used herein, the phrase “identified to be in need of treatment for a disease or disorder,” or the like, refers to selection of a subject based upon need for treatment of the disease or disorder. For example, a subject can be identified as having a need for treatment of a disease or disorder (e.g., such as a GSD or Pompe Disease) based upon an earlier diagnosis by a person of skill and thereafter subjected to treatment for the genetic disease or disorder. In an aspect, the identification can be performed by a person different from the person making the diagnosis. In an aspect, the administration can be performed by one who performed the diagnosis.

As used herein, “inhibit,” “inhibiting”, and “inhibition” mean to diminish or decrease an activity, level, response, condition, severity, disease, or other biological parameter. This can include, but is not limited to, the complete ablation of the activity, level, response, condition, severity, disease, or other biological parameter. This can also include, for example, a 10% inhibition or reduction in the activity, level, response, condition, severity, disease, or other biological parameter as compared to the native or control level (e.g., a subject not having a disease or disorder like Pompe disease or a genetic disease or disorder). Thus, in an aspect, the inhibition or reduction can be a 10%, 20%, 30%, 40%, 50%, 60%, 70%, 80%, 90%, 100%, or any amount of reduction in between as compared to native or control levels. In an aspect, the inhibition or reduction can be 10-20%, 20-30%, 30-40%, 40-50%, 50-60%, 60-70%, 70-80%, 80-90%, or 90-100% as compared to native or control levels. In an aspect, the inhibition or reduction can be 0-25%, 25-50%, 50-75%, or 75-100% as compared to native or control levels. In an aspect, a native or control level can be a pre-disease or pre-disorder level.

The words “treat” or “treating” or “treatment” include palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In an aspect, the terms cover any treatment of a subject, including a mammal (e.g., a human), and includes: (i) preventing the undesired physiological change, disease, pathological condition, or disorder from occurring in a subject that can be predisposed to the disease but has not yet been diagnosed as having it; (ii) inhibiting the physiological change, disease, pathological condition, or disorder, i.e., arresting its development; or (iii) relieving the physiological change, disease, pathological condition, or disorder, i.e., causing regression of the disease. For example, in an aspect, treating a disease or disorder can reduce the severity of an established a disease or disorder in a subject by 1%-100% as compared to a control (such as, for example, an individual not having Pompe disease or a genetic disease or disorder). In an aspect, treating can refer to a 1%, 2%, 3%, 4%, 5%, 6%, 7%, 8%, 9%, 10%, 20%, 30%, 40%, 50%, 60%, 70%, 80%, 90%, or 100% reduction in the severity of a disease or disorder (such as a genetic disease or disorder). For example, treating a disease or disorder can reduce one or more symptoms of a disease or disorder in a subject by 1%-100% as compared to a control (such as, for example, an individual not having a genetic disease or disorder). In an aspect, treating can refer to 1%, 2%, 3%, 4%, 5%, 6%, 7%, 8%, 9%, 10%, 20%, 30%, 40%, 50%, 60%, 70%, 80%, 90%, 100% reduction of one or more symptoms of an established a disease or disorder (such as Pompe disease). It is understood that treatment does not necessarily refer to a cure or complete ablation or eradication of a disease or disorder. However, in an aspect, treatment can refer to a cure or complete ablation or eradication of a disease or disorder.

As used herein, the term “prevent” or “preventing” or “prevention” refers to precluding, averting, obviating, forestalling, stopping, or hindering something from happening, especially by advance action. It is understood that where reduce, inhibit, or prevent are used herein, unless specifically indicated otherwise, the use of the other two words is also expressly disclosed. In an aspect, preventing a disease or disorder having chromatin deregulation and/or chromatin dysregulation is intended. The words “prevent”, “preventing”, and “prevention” also refer to prophylactic or preventative measures for protecting or precluding a subject (e.g., an individual) not having a given a disease or disorder (such as Pompe disease or a genetic disease or disorder) or a related complication from progressing to that complication.

As used herein, the terms “administering” and “administration” refer to any method of providing one or more of the disclosed isolated nucleic acid molecules, disclosed vectors, disclosed pharmaceutical formulations, or a combination thereof to a subject. Such methods are well known to those skilled in the art and include, but are not limited to, the following: oral administration, transdermal administration, administration by inhalation, nasal administration, topical administration, in utero administration, intrahepatic administration, intravaginal administration, ophthalmic administration, intraaural administration, otic administration, intracerebral administration, rectal administration, sublingual administration, buccal administration, and parenteral administration, including injectable such as intravenous administration, intra-CSF administration, intra-arterial administration, intramuscular administration, and subcutaneous administration. Administration can also include hepatic intra-arterial administration or administration through the hepatic portal vein (HPV). Administration of a disclosed isolated nucleic acid molecule, a disclosed vector, a disclosed pharmaceutical composition, a disclosed therapeutic agent, a disclosed immune modulator, a disclosed proteasome inhibitor, a disclosed small molecule, and/or a disclosed endonuclease can comprise administration directly into the CNS or the PNS. Administration can be continuous or intermittent. Administration can comprise a combination of one or more route.

In an aspect, the skilled person can determine an efficacious dose, an efficacious schedule, and an efficacious route of administration for one or more of the disclosed isolated nucleic acid molecules, disclosed vectors, disclosed pharmaceutical formulations, or a combination thereof to treat or prevent a disease or disorder (such as genetic disease or disorder). In an aspect, the skilled person can also alter, change, or modify an aspect of an administering step to improve efficacy of one or more of the disclosed isolated nucleic acid molecules, disclosed vectors, disclosed pharmaceutical formulations, or a combination thereof.

By “determining the amount” is meant both an absolute quantification of a particular analyte (e.g., an mRNA sequence containing a particular tag) or a determination of the relative abundance of a particular analyte (e.g., an amount as compared to a mRNA sequence including a different tag). The phrase includes both direct or indirect measurements of abundance (e.g., individual mRNA transcripts may be quantified or the amount of amplification of an mRNA sequence under certain conditions for a certain period may be used a surrogate for individual transcript quantification) or both.

As used herein, “modifying the method” can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, by changing the duration of time one or more of the disclosed isolated nucleic acid molecules, disclosed vectors, disclosed pharmaceutical formulations, or a combination are administered to a subject, or by substituting for one or more of the disclosed components and/or reagents with a similar or equivalent component and/or reagent. The same applies to all disclosed therapeutic agents, immune modulators, immunosuppressive agents, proteosome inhibitors, etc.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents, or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. In an aspect, a pharmaceutical carrier employed can be a solid, liquid, or gas. In an aspect, examples of solid carriers can include lactose, terra alba, sucrose, talc, gelatin, agar, pectin, acacia, magnesium stearate, and stearic acid. In an aspect, examples of liquid carriers can include sugar syrup, peanut oil, olive oil, and water. In an aspect, examples of gaseous carriers can include carbon dioxide and nitrogen. In preparing a disclosed composition for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

As used herein, the term “excipient” refers to an inert substance which is commonly used as a diluent, vehicle, preservative, binder, or stabilizing agent, and includes, but is not limited to, proteins (e.g., serum albumin, etc.), amino acids (e.g., aspartic acid, glutamic acid, lysine, arginine, glycine, histidine, etc.), fatty acids and phospholipids (e.g., alkyl sulfonates, caprylate, etc.), surfactants (e.g., SDS, polysorbate, nonionic surfactant, etc.), saccharides (e.g., sucrose, maltose, trehalose, etc.) and polyols (e.g., mannitol, sorbitol, etc.). See, also, for reference, Remington's Pharmaceutical Sciences, (1990) Mack Publishing Co., Easton, Pa., which is hereby incorporated by reference in its entirety.

As used herein, “concurrently” means (1) simultaneously in time, or (2) at different times during the course of a common treatment schedule.

“Efficiency” when used in describing viral production, replication or packaging refers to useful properties of the method: in particular, the growth rate and the number of virus particles produced per cell. “High efficiency” production indicates production of at least 100 viral particles per cell; e.g., at least about 10,000 or at least about 100,000 particles per cell, over the course of the culture period specified.

The term “contacting” as used herein refers to bringing one or more of the disclosed isolated nucleic acid molecules, disclosed vectors, disclosed pharmaceutical formulations, or a combination thereof together with a target area or intended target area in such a manner that the one or more of the disclosed isolated nucleic acid molecules, disclosed vectors, disclosed pharmaceutical formulations, or a combination thereof exert an effect on the intended target or targeted area either directly or indirectly. A target area can comprise one or more cells, and in an aspect, one or more cells can be in a subject. A target area or intended target area can be one or more of a subject's organs (e.g., lungs, heart, liver, kidney, brain, etc.). In an aspect, a target area or intended target area can be any cell or any organ infected by a disease or disorder (such as a genetic disease or disorder). In an aspect, a target area or intended target area can be any organ, tissue, or cells that are affected by a disease or disorder (such as Pompe disease or a genetic disease or disorder).

As used herein, “determining” can refer to measuring or ascertaining the presence and severity of a disease or disorder, such as, for example, a genetic disease or disorder. Methods and techniques used to determine the presence and/or severity of a disease or disorder are typically known to the medical arts. For example, the art is familiar with the ways to identify and/or diagnose the presence, severity, or both of a disease or disorder (such as, for example, a genetic disease or disorder).

As used herein, “effective amount” and “amount effective” can refer to an amount that is sufficient to achieve the desired result such as, for example, the treatment and/or prevention of a disease or disorder (e.g., a genetic disease or disorder such as Pompe disease) or a suspected disease or disorder. As used herein, the terms “effective amount” and “amount effective” can refer to an amount that is sufficient to achieve the desired an effect on an undesired condition (e.g., a disease or disorder such as Pompe disease). For example, a “therapeutically effective amount” refers to an amount that is sufficient to achieve the desired therapeutic result or to have an effect on undesired symptoms, but is generally insufficient to cause adverse side effects. In an aspect, “therapeutically effective amount” means an amount of a disclosed isolated nucleic acid molecule, a disclosed vector, or a disclosed pharmaceutical formulation; that (i) treats the particular disease, condition, or disorder (e.g., a genetic disease or disorder such as Pompe disease), (ii) attenuates, ameliorates, or eliminates one or more symptoms of the particular disease, condition, or disorder e.g., a genetic disease or disorder such as Pompe disease), or (iii) delays the onset of one or more symptoms of the particular disease, condition, or disorder described herein (e.g., a genetic disease or disorder such as Pompe disease). The specific therapeutically effective dose level for any particular patient will depend upon a variety of factors including the disorder being treated and the severity of the disorder; the disclosed isolated nucleic acid molecules, disclosed vectors, disclosed pharmaceutical formulations employed; the disclosed methods employed; the age, body weight, general health, sex and diet of the patient; the time of administration; the route of administration; the rate of excretion of the disclosed isolated nucleic acid molecules, disclosed vectors, or disclosed pharmaceutical formulations employed; the duration of the treatment; drugs used in combination or coincidental with the disclosed isolated nucleic acid molecules, disclosed vectors, or disclosed pharmaceutical formulations employed, and other like factors well known in the medical arts. For example, it is well within the skill of the art to start doses of the disclosed isolated nucleic acid molecules, disclosed vectors, or disclosed pharmaceutical formulations at levels lower than those required to achieve the desired therapeutic effect and to gradually increase the dosage until the desired effect is achieved. If desired, then the effective daily dose can be divided into multiple doses for purposes of administration. Consequently, a single dose of the disclosed isolated nucleic acid molecules, disclosed vectors, or disclosed pharmaceutical formulations can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition, such as, for example, a disease or disorder due to a missing, deficient, and/or mutant protein or enzyme.

As used herein, “expression cassette” or “transgene cassette” can refer to a distinct component of vector DNA comprising a transgene and one or more regulatory sequences to be expressed by a transfected cell. Generally, an expression cassette or transgene cassette can comprise a promoter sequence, an open reading frame (i.e., the transgene such as, for example, an GAA or a portion thereof), and a 3′ untranslated region (e.g., a polyadenylation site).

As used herein, “small molecule” can refer to any organic or inorganic material that is not a polymer. Small molecules exclude large macromolecules, such as large proteins (e.g., proteins with molecular weights over 2,000, 3,000, 4,000, 5,000, 6,000, 7,000, 8,000, 9,000, or 10,000), large nucleic acids (e.g., nucleic acids with molecular weights of over 2,000, 3,000, 4,000, 5,000, 6,000, 7,000, 8,000, 9,000, or 10,000), or large polysaccharides (e.g., polysaccharides with a molecular weight of over 2,000, 3,000, 4,000, 5,000, 6,000, 7,000, 8,000, 9,000, or 10,000). In an aspect, a “small molecule”, for example, can be a drug that can enter cells easily because it has a low molecular weight. In an aspect, a small molecule can be used in conjunction with a disclosed composition in a disclosed method.

As used herein, “homology directed repair” or “HDR” can occur either non-conservatively or conservatively. The non-conservative method is composed of the single-strand annealing (SSA) pathway and is more error prone. The conservative methods, characterized by the accurate repair of the DSB by means of a homologous donor (e.g., sister chromatid, plasmid, etc.), are composed of three pathways: the classical double-strand break repair (DSBR), synthesis-dependent strand-annealing (SDSA), and break-induced repair (BIR). For example, in the classical DSBR pathway, the 3′ ends invade an intact homologous template to serve as a primer for DNA repair synthesis, ultimately leading to the formation of double Holliday junctions (dHJs). dHJs are four-stranded branched structures that form when elongation of the invasive strand “captures” and synthesizes DNA from the second DSB end. The individual HJs are resolved via cleavage in one of two ways. Each junction resolution could happen on the crossing strand (horizontally at the purple arrows) or on the non-crossing strand (vertically at the orange arrows). If resolved dissimilarly (e.g., one junction is resolved on the crossing strand and the other on the non-crossing strand), then a crossover event will occur; however, if both HJs are resolved in the same manner, this results in a non-crossover event. DSBR is semi-conservative, as crossover events are most common.

As used herein, “transduction” or “transducing” are terms referring to a process for the introduction of an exogenous polynucleotide, e.g., a transgene in rAAV vector, into a host cell leading to expression of the polynucleotide, e.g., the transgene in the cell. The process can include one or more of (i) endocytosis of the chimeric virus, (ii) escape from endosomes or other intracellular compartments in the cytosol of a cell, (iii) trafficking of the viral particle or viral genome to the nucleus, (iv) uncoating of the virus particles, and (v) generation of expressible double stranded AAV genome forms, including circular intermediates. The rAAV expressible double stranded form may persist as a nuclear episome or optionally may integrate into the host genome. Methods used for the introduction of the exogenous polynucleotide include well-known techniques such as transfection, lipofection, viral infection, transformation, and electroporation, as well as non-viral gene delivery techniques. The introduced polynucleotide may be stably or transiently maintained in the host cell.

As used herein, “operably linked” means that expression of a gene or a transgene is under the control of a promoter with which it is spatially connected. A promoter can be positioned 5′ (upstream) or 3′ (downstream) of a gene under its control. The distance between the promoter and a gene can be approximately the same as the distance between that promoter and the gene it controls in the gene from which the promoter is derived. As is known in the art, variation in this distance can be accommodated without loss of promoter function.

As used herein, “peptide,” “polypeptide,” and “protein” are used interchangeably, and refer to a compound comprised of amino acid residues covalently linked by peptide bonds. A protein must contain at least two amino acids and there is no limitation on the maximum number of amino acids that can comprise a protein's sequence. The term “peptide” can refer to a short chain of amino acids including, for example, natural peptides, recombinant peptides, synthetic peptides, or any combination thereof. Proteins and peptides can include, for example, biologically active fragments, substantially homologous polypeptides, oligopeptides, homodimers, heterodimers, variants of polypeptides, modified polypeptides, derivatives, analogs, and fusion proteins, among others.

A cell is said to be “stably” altered, transduced or transformed with a genetic sequence if the sequence is available to perform its function during extended culture of the cell in vitro. In some examples, such a cell is “inheritably” altered in that a genetic alteration is introduced which is also inheritable by progeny of the altered cell.

“Nucleic acid” or “oligonucleotide” or “polynucleotide” as used herein means at least two nucleotides covalently linked together. The depiction of a single strand can also define the sequence of the complementary strand. Thus, a nucleic acid can encompass the complementary strand of a depicted single strand. Many variants of a nucleic acid can be used for the same purpose as a given nucleic acid. Thus, a nucleic acid can encompass substantially identical nucleic acids and complements thereof. A single strand can provide a probe that can hybridize to a target sequence under stringent hybridization conditions. Thus, a nucleic acid can encompass a probe that hybridizes under stringent hybridization conditions. A nucleic acid can be single-stranded, or double-stranded, or can contain portions of both double-stranded and single-stranded sequence. The nucleic acid can be DNA, both genomic and cDNA, RNA, or a hybrid, where the nucleic acid can contain combinations of deoxyribo- and ribo-nucleotides, and combinations of bases including uracil, adenine, thymine, cytosine, guanine, inosine, xanthine hypoxanthine, isocytosine and isoguanine. Nucleic acids can be obtained by chemical synthesis methods or by recombinant methods. Also as used herein, the terms “nucleic acid,” “nucleic acid molecule,” “nucleic acid construct,” “nucleotide sequence”, and “polynucleotide” can refer to RNA or DNA that is linear or branched, single or double stranded, or a hybrid thereof. The term can encompass RNA/DNA hybrids. When dsRNA is produced synthetically, less common bases, such as inosine, 5-methylcytosine, 6-methyladenine, hypoxanthine and others can also be used for antisense, dsRNA, and ribozyme pairing. For example, polynucleotides that contain C-5 propyne analogues of uridine and cytidine have been shown to bind RNA with high affinity and to be potent antisense inhibitors of gene expression. Other modifications, such as modification to the phosphodiester backbone, or the 2′-hydroxy in the ribose sugar group of the RNA can also be made. A “synthetic” nucleic acid or polynucleotide, as used herein, refers to a nucleic acid or polynucleotide that is not found in nature but is constructed by the hand of man and therefore is not a product of nature.

A “polynucleotide” is a sequence of nucleotide bases, and may be RNA, DNA, or DNA-RNA hybrid sequences (including both naturally occurring and non-naturally occurring nucleotides).

A “fragment” or “portion” of a nucleotide sequence can be understood to mean a nucleotide sequence of reduced length relative (e.g., reduced by 1, 2, 3, 4, 5, 6, 7, 8, 9, 10, 11, 12, 13, 14, 15, 16, 17, 18, 19, 20 or more nucleotides) to a reference nucleic acid or nucleotide sequence and comprising, consisting essentially of, or consisting of a nucleotide sequence of contiguous nucleotides identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference nucleic acid or nucleotide sequence. Such a nucleic acid fragment or portion according to the disclosure can be, where appropriate, included in a larger polynucleotide of which it is a constituent. In an aspect, a fragment or portion of a nucleotide sequence or nucleic acid sequence can comprise the sequence encoding an exon having one or more mutations.

A “fragment” or “portion” of an amino acid sequence can be understood to mean an amino acid sequence of reduced length relative (e.g., reduced by 1, 2, 3, 4, 5, 6, 7, 8, 9, 10, 11, 12, 13, 14, 15, 16, 17, 18, 19, 20, 30, 40, 50, 60, 70, 80, 90, 100, 150, 200, 250, or more amino acids) to a reference amino acid sequence and comprising, consisting essentially of, or consisting of an amino acid sequence of contiguous amino acids identical or almost identical (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% identical) to the reference amino acid sequence. Such an amino acid fragment or portion according to the disclosure can be, where appropriate, included in a larger amino acid sequence of which it is a constituent.

A “heterologous” or a “recombinant” nucleotide or amino acid sequence as used interchangeably herein can refer to a nucleotide or an amino acid sequence not naturally associated with a host cell into which it is introduced, including non-naturally occurring multiple copies of a naturally occurring nucleotide or amino acid sequence.

“Complement” or “complementary” as used herein means a nucleic acid can mean Watson-Crick (e.g., A-T/U and C-G) or Hoogsteen base pairing between nucleotides or nucleotide analogs of nucleic acid molecules. “Complementarity” refers to a property shared between two nucleic acid sequences, such that when they are aligned antiparallel to each other, the nucleotide bases at each position will be complementary.

As used herein, “promoter” or “promoters” are known to the art. Depending on the level and tissue-specific expression desired, a variety of promoter elements can be used. A promoter can be tissue-specific or ubiquitous and can be constitutive or inducible, depending on the pattern of the gene expression desired. A promoter can be native (endogenous) or foreign (exogenous) and can be a natural or a synthetic sequence. By foreign or exogenous, it is intended that the transcriptional initiation region is not found in the wild-type host into which the transcriptional initiation region is introduced.

“Tissue-specific promoters” are known to the art and include, but are not limited to, neuron-specific promoters, muscle-specific promoters, liver-specific promoters, skeletal muscle-specific promoters, and heart-specific promoters.

“Liver-specific promoters” are known to the art and include, but are not limited to, the thyroxin binding globulin (TBG) promoter, the α1-microglobulin/bikunin enhancer/thyroid hormone-binding globulin promoter, the human albumin (hALB) promoter, the thyroid hormone-binding globulin promoter, the α-1-anti-trypsin promoter, the bovine albumin (bAlb) promoter, the murine albumin (mAlb) promoter, the human α1-antitrypsin (hAAT) promoter, the ApoEhAAT promoter comprising the ApoE enhancer and the hAAT promoter, the transthyretin (TTR) promoter, the liver fatty acid binding protein promoter, the hepatitis B virus (HBV) promoter, the DC172 promoter comprising the hAAT promoter and the α1-microglobulin enhancer, the DC190 promoter comprising the human albumin promoter and the prothrombin enhancer, or any other natural or synthetic liver-specific promoter. In an aspect, a liver specific promoter can comprise about 845-bp and comprise the thyroid hormone-binding globulin promoter sequences (2382 to 13), two copies of α1-microglobulin/bikunin enhancer sequences (22,804 through 22,704), and a 71-bp leader sequence as described by Ill C R, et al. (1997).

Ubiquitous/constitutive promoters” are known to the art and include, but are not limited to, a CMV major immediate-early enhancer/chicken beta-actin promoter, a cytomegalovirus (CMV) major immediate-early promoter, an Elongation Factor 1-α (EF1-α) promoter, a simian vacuolating virus 40 (SV40) promoter, an AmpR promoter, a PγK promoter, a human ubiquitin C gene (Ubc) promoter, a MFG promoter, a human beta actin promoter, a CAG promoter, a EGR1 promoter, a FerH promoter, a FerL promoter, a GRP78 promoter, a GRP94 promoter, a HSP70 promoter, a β-kin promoter, a murine phosphoglycerate kinase (mPGK) or human PGK (hPGK) promoter, a ROSA promoter, human Ubiquitin B promoter, a Rous sarcoma virus promoter, or any other natural or synthetic ubiquitous/constitutive promoters.

As used herein, an “inducible promoter” refers to a promoter that can be regulated by positive or negative control. Factors that can regulate an inducible promoter include, but are not limited to, chemical agents (e.g., the metallothionein promoter or a hormone inducible promoter), temperature, and light.

As used herein, the term “serotype” is a distinction used to refer to an AAV having a capsid that is serologically distinct from other AAV serotypes. Serologic distinctiveness can be determined by the lack of cross-reactivity between antibodies to one AAV as compared to another AAV. Such cross-reactivity differences are usually due to differences in capsid protein sequences/antigenic determinants (e.g., due to VP1, VP2, and/or VP3 sequence differences of AAV serotypes).

As used herein, “tropism” refers to the specificity of an AAV capsid protein present in an AAV viral particle, for infecting a particular type of cell or tissue. The tropism of an AAV capsid for a particular type of cell or tissue may be determined by measuring the ability of AAV vector particles comprising the hybrid AAV capsid protein to infect or to transduce a particular type of cell or tissue, using standard assays that are well-known in the art such as those disclosed in the examples of the present application. As used herein, the term “liver tropism” or “hepatic tropism” refers to the tropism for liver or hepatic tissue and cells, including hepatocytes.

“Sequence identity” and “sequence similarity” can be determined by alignment of two peptide or two nucleotide sequences using global or local alignment algorithms. Sequences may then be referred to as “substantially identical” or “essentially similar” when they are optimally aligned. For example, sequence similarity or identity can be determined by searching against databases such as FASTA, BLAST, etc., but hits should be retrieved and aligned pairwise to compare sequence identity. Two proteins or two protein domains, or two nucleic acid sequences can have “substantial sequence identity” if the percentage sequence identity is at least 70%, 75%, 80%, 85%, 90%, 95%, 98%, 99% or more, preferably 90%, 95%, 98%, 99% or more. Such sequences are also referred to as “variants” herein, e.g., other variants of a missing, deficient, and/or mutant protein or enzyme. It should be understood that sequence with substantial sequence identity do not necessarily have the same length and may differ in length. For example, sequences that have the same nucleotide sequence but of which one has additional nucleotides on the 3′- and/or 5′-side are 100% identical.

As used herein, “codon optimization” can refer to a process of modifying a nucleic acid sequence for enhanced expression in the host cells of interest by replacing one or more codons or more of the native sequence with codons that are more frequently or most frequently used in the genes of that host cell while maintaining the native amino acid sequence. Various species exhibit particular bias for certain codons of a particular amino acid. As contemplated herein, genes can be tailored for optimal gene expression in a given organism based on codon optimization. Codon usage tables are readily available, for example, at the “Codon Usage Database.” Many methods and software tools for codon optimization have been reported previously. (See, for example, genomes.urv.es/OPTIMIZER/).

As used herein, “RNA Editing” can refer to a type of genetic engineering in which an RNA molecule (or ribonucleotides of the RNA) is inserted, deleted, or replaced in the genome of an organism using engineered nucleases, which create site-specific strand breaks at desired locations in the RNA. The induced breaks are repaired resulting in targeted mutations or repairs.

As used herein, “CRISPR or clustered regularly interspaced short palindromic repeat” is an ideal tool for correction of genetic abnormalities as the system can be designed to target genomic DNA directly. Cas9 is well-known to the art. SaCas9 is well-known to the art (see, e.g., SEQ ID NO:19). The CRISPR/Cas methods disclosed herein, such as those that use a Cas9 or SaCas9. can be used to edit the sequence of one or more target RNAs, such as one associated with a disease or disorder disclosed herein (e.g., a genetic disease or disorder such as Pompe disease). For example, in an aspect, a nuclease mediated break in the stem cell DNA can allow for the insertion of one or multiple genes via homology directed repair.

As used herein, “immune tolerance,” “immunological tolerance,” and “immunotolerance” refers to a state of unresponsiveness or blunted response of the immune system to substances (e.g., a disclosed isolated nucleic acid molecule, a disclosed vector, a disclosed transgene product, a disclosed pharmaceutical formulation, a disclosed therapeutic agent, etc.) that have the capacity to elicit an immune response in a subject. Immune tolerance is induced by prior exposure to a specific antigen. Immune tolerance can be determined in a subject by measuring antibodies against a particular antigen or by liver-restricted transgene expression with a viral vector (such as, for example, AAV). Low or absent antibody titers over time is an indicator of immune tolerance.

For example, in some embodiments, immune tolerance can be established by having IgG antibody titers of less than or equal to about 12,000, 11,500, 11,000, 10,500, 10,000, 9,500, 9,000, 8,500, 8,000, 7,500, 7,000, 6,500, or 6,000 within following gene therapy (such as the administration of the transgene encoding, for example, a missing, deficient, and/or mutant protein or enzyme).

As known to the art, antibodies (Abs) can mitigate AAV infection through multiple mechanisms by binding to AAV capsids and blocking critical steps in transduction such as cell surface attachment and uptake, endosomal escape, productive trafficking to the nucleus, or uncoating as well as promoting AAV opsonization by phagocytic cells, thereby mediating their rapid clearance from the circulation. For example, in humans, serological studies reveal a high prevalence of NAbs in the worldwide population, with about 67% of people having antibodies against AAV1, 72% against AAV2, and approximately 40% against AAV serotypes 5 through 9. Vector immunogenicity represents a major challenge in re-administration of AAV vectors.

In an aspect, also disclosed herein are partial self-complementary parvovirus (e.g., a disclosed AAV) genomes, plasmid vectors encoding the parvovirus genomes, and parvovirus (e.g., a disclosed AAV) particles including such genomes. In an aspect, provided herein is a plasmid vector comprising a nucleotide sequence encoding a disclosed parvovirus genome such as for example, a disclosed AAV. In an aspect, provided herein is a partial self-complementary parvovirus genome including a payload construct, parvovirus ITRs flanking the payload construct, and a self-complementary region flanking one of the ITRs. A self-complementary region can comprise a nucleotide sequence that is complementary to the payload construct. A disclosed self-complementary region can have a length that is less the entire length of the payload construct.

In an aspect, a disclosed self-complementary region of a disclosed parvovirus genome can comprise a minimum length, while still having a length that is less the entire length of the payload construct. In an aspect, a disclosed self-complementary region can comprise at least 50 bases in length, at least 100 bases in length, at least 200 in length, at least 300 bases in length, at least 400 bases in length, at least 500 bases in length, at least 600 bases in length, at least 700 bases in length, at least 800 bases in length, at least 900 bases in length, or at least 1,000 bases in length.

In an aspect, a “self-complementary parvovirus genome” can be a single stranded polynucleotide having, in the 5′ to 3′ direction, a first parvovirus ITR sequence, a heterologous sequence (e.g., payload construct comprising, for example, a desired gene), a second parvovirus ITR sequence, a second heterologous sequence, wherein the second heterologous sequence is complementary to the first heterologous sequence, and a third parvovirus ITR sequence. In contrast to a self-complementary genome, a “partial self-complementary genome” does not include three parvovirus ITRs and the second heterologous sequence that is complementary to the first heterologous sequence has a length that is less than the entire length of the first heterologous sequence (e.g., payload construct). Accordingly, a partial self-complementary genome is a single stranded polynucleotide having, in the 5′ to 3′ direction or the 3′ to 5′ direction, a first parvovirus ITR sequence, a heterologous sequence (e.g., payload construct), a second parvovirus ITR sequence, and a self-complementary region that is complementary to a portion of the heterologous sequence and has a length that is less than the entire length the heterologous sequence.

As used herein, “immune-modulating” refers to the ability of a disclosed isolated nucleic acid molecules, a disclosed vector, a disclosed pharmaceutical formulation, or a disclosed agent to alter (modulate) one or more aspects of the immune system. The immune system functions to protect the organism from infection and from foreign antigens by cellular and humoral mechanisms involving lymphocytes, macrophages, and other antigen-presenting cells that regulate each other by means of multiple cell-cell interactions and by elaborating soluble factors, including lymphokines and antibodies, that have autocrine, paracrine, and endocrine effects on immune cells.

As used herein, “immune modulator” refers to an agent that is capable of adjusting a given immune response to a desired level (e.g., as in immunopotentiation, immunosuppression, or induction of immunologic tolerance). Examples of immune modulators include but are not limited to, a disclosed immune modulator can comprise aspirin, azathioprine, belimumab, betamethasone dipropionate, betamethasone valerate, bortezomib, bredinin, cyazathioprine, cyclophosphamide, cyclosporine, deoxyspergualin, didemnin B, fluocinolone acetonide, folinic acid, ibuprofen, IL6 inhibitors (such as sarilumab) indomethacin, inebilizumab, intravenous gamma globulin (IVIG), methotrexate, methylprednisolone, mycophenolate mofetil, naproxen, prednisolone, prednisone, prednisolone indomethacin, rapamycin, rituximab, sirolimus, sulindac, synthetic vaccine particles containing rapamycin (SVP-Rapamycin or ImmTOR), thalidomide, tocilizumab, tolmetin, triamcinolone acetonide, anti-CD3 antibodies, anti-CD4 antibodies, anti-CD19 antibodies, anti-CD20 antibodies, anti-CD22 antibodies, anti-CD40 antibodies, anti-FcRN antibodies, anti-IL6 antibodies, anti-IGF1R antibodies, an IL2 mutein, a BTK inhibitor, or a combination thereof. In an aspect, a disclosed immune modulator can comprise one or more Treg (regulatory T cells) infusions (e.g., antigen specific Treg cells to AAV). In an aspect, a disclosed immune modulator can be bortezomib or SVP-Rapamycin. In an aspect, an immune modulator can be administered by any suitable route of administration including, but not limited to, in utero, intra-CSF, intrathecally, intravenously, subcutaneously, transdermally, intradermally, intramuscularly, orally, transcutaneously, intraperitoneally (IP), or intravaginally. In an aspect, a disclosed immune modulator can be administered using a combination of routes. Administration can also include hepatic intra-arterial administration or administration through the hepatic portal vein (HPV). Administration of an immune modulator can be continuous or intermittent, and administration can comprise a combination of one or more routes.

As used herein, the term “immunotolerant” refers to unresponsiveness to an antigen (e.g., a vector, a therapeutic protein, a transgene product, etc.). An immunotolerant promoter can reduce, ameliorate, or prevent transgene-induced immune responses that can be associated with gene therapy. Assays known in the art to measure immune responses, such as immunohistochemical detection of cytotoxic T cell responses, can be used to determine whether one or more promoters can confer immunotolerant properties.

As used herein, the term “package insert” is used to refer to instructions customarily included in commercial packages of therapeutic products, that contain information about the indications, usage, dosage, administration, contraindications and/or warnings concerning the use of such therapeutic products.

As used herein, the term “in combination” in the context of the administration of other therapies (e.g., other agents) includes the use of more than one therapy (e.g., drug therapy). Administration “in combination with” one or more further therapeutic agents includes simultaneous (e.g., concurrent) and consecutive administration in any order. The use of the term “in combination” does not restrict the order in which therapies are administered to a subject. By way of non-limiting example, a first therapy (e.g., a disclosed isolated nucleic acid molecule, a disclosed vector, a disclosed pharmaceutical formulation, or a combination thereof) may be administered prior to (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 8 weeks, 8 weeks, 9 weeks, 10 weeks, 11 weeks, or 12 weeks), concurrently, or after (e.g., 1 minute, 15 minutes, 30 minutes, 45 minutes, 1 hour, 2 hours, 3 hours, 4 hours, 5 hours, 6 hours, 7 hours, 8 hours, 12 hours, 24 hours, 48 hours, 72 hours, 96 hours, 1 week, 2 weeks, 3 weeks, 4 weeks, 5 weeks, 6 weeks, 7 weeks, 8 weeks, 9 weeks, 10 weeks, 11 weeks, or 12 weeks or longer) the administration of a second therapy (e.g., agent) to a subject having or diagnosed with a disease or disorder (such as a genetic disease or disorder).

Disclosed are the components to be used to prepare the disclosed isolated nucleic acid molecules, disclosed vectors, or disclosed pharmaceutical formulations as well as the disclosed isolated nucleic acid molecules, disclosed vectors, or disclosed pharmaceutical formulations used within the methods disclosed herein. These and other materials are disclosed herein, and it is understood that when combinations, subsets, interactions, groups, etc. of these materials are disclosed that while specific reference of each various individual and collective combinations and permutation of these compounds cannot be explicitly disclosed, each is specifically contemplated and described herein. For example, if a particular compound is disclosed and discussed and a number of modifications that can be made to a number of molecules including the compounds are discussed, specifically contemplated is each and every combination and permutation of the compound and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the compositions of the invention. Thus, if there are a variety of additional steps that can be performed it is understood that each of these additional steps can be performed with any specific embodiment or combination of embodiments of the methods of the invention.

### B. Compositions for Gene Editing

### 1. Nucleic Acid Molecules

Disclosed herein is an isolated nucleic acid molecule, comprising: a nucleic acid sequence encoding a repair template for Pompe disease.

In an aspect, a disclosed repair template can be for the IVS1 variant of Pompe disease, for the ΔT525 variant of Pompe disease, or for the 1826DupA variant of Pompe disease. In an aspect, a disclosed repair template can be for any mutation in the gene encoding alpha glucosidase (GAA) or can be for one or more mutations in the gene encoding alpha glucosidase (GAA).

In an aspect, a disclosed repair template comprises about 300 bp to about 700 bp. In an aspect, a disclosed repair template can comprise about 400 bp, or about 600 bp, or about 650 bp.

In an aspect, a disclosed repair template can comprise the sequence set forth in SEQ ID NO: 26, SEQ ID NO:27, and SEQ ID NO:28, or a sequence having about 50%, about 60%, about 70% about 80%, about 90%, about 95%, or more identity to the sequence set forth in SEQ ID NO: 26, SEQ ID NO:27, and SEQ ID NO:28. In an aspect, a disclosed repair template can comprise a nucleic acid sequence encoding wild-type GAA or a portion thereof. In an aspect, a disclosed GAA can comprise the sequence set forth in SEQ ID NO:62, or a sequence having about 50%, about 60%, about 70% about 80%, about 90%, about 95%, or more identity to the sequence set forth in SEQ ID NO:62.

In an aspect, a disclosed repair template can comprise a restriction site. Restriction enzymes are known to the art.

For example, a disclosed repair template can comprise a first sequence having homology to a sequence in intron 1 of GAA and a second sequence having homology to a sequence in exon 2 of GAA. In an aspect, a disclosed first homologous sequence can comprise the sequence of a wild-type GAA or a portion thereof, and a disclosed second homologous sequence can comprise the sequence of a wild-type GAA or a portion thereof, or any combination thereof. In an aspect, a disclosed GAA can comprise the sequence set forth in SEQ ID NO:62.

In an aspect, a disclosed repair template can comprise a promoter sequence between the first homologous sequence and the second homology sequence. In an aspect, a disclosed promoter can comprise a CMV promoter, a EF1a promoter, a chicken beta-actin promoter, a liver-specific promoter, or a G6PC promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect, a disclosed first homologous sequence can comprise about 175 bp to about 225 bp or about 200 bp to about 220 bp. For example, in an aspect, a disclosed first homologous sequence can comprise the sequence set forth in SEQ ID NO:54, SEQ ID NO:55, SEQ ID NO:56, or SEQ ID NO:57. In an aspect, a disclosed second homologous sequence can comprise about 175 bp to about 225 bp or about 200 bp to about 220 bp. In an aspect, a disclosed second homologous can comprise the sequence set forth in SEQ ID NO:58, SEQ ID NO:59, SEQ ID NO: 60, or SEQ ID NO:61. In an aspect, a disclosed first homologous sequence can comprise the sequence of a wild-type GAA or a portion thereof, a disclosed second homologous sequence can comprise the sequence of a wild-type GAA or a portion thereof. In an aspect, a disclosed GAA can comprise the sequence set forth in SEQ ID NO:62.

In an aspect, a disclosed repair template can comprise a sequence having homology to a sequence in exon 2 of GAA. In an aspect, a disclosed repair template can comprise a sequence having homology to a sequence in exon 13 of GAA.

In an aspect, a disclosed isolated nucleic acid molecule can comprise a nucleic acid sequence encoding a promoter that is operably linked to a nucleic acid sequence encoding a guide RNA (gRNA) and drives the expression of the gRNA. In an aspect, a disclosed gRNA can target a PAM sequence at or near the IVS1 variant near the boundary between intron 1 and exon 2 in the GAA gene, a PAM sequence at or near the ΔT525 variant in Exon 2 in the GAA gene, or a PAM sequence at or near the m1826DupA variant in Exon 13 in the GAA gene. In an aspect, a disclosed gRNA can target a PAM sequence at or near any mutation in the GAA gene.

In an aspect, a disclosed gRNA can comprise the sequence set forth in any of SEQ ID NO: 29-SEQ ID NO:36.

In an aspect, a disclosed promoter operably linked to a disclosed gRNA can comprise a RNA polymerase III (Poly III) promoter. In an aspect, a disclosed Poly III promoter can comprise a U3 promoter, a U6 promoter, or a H1 promoter. In an aspect, a disclosed promoter operably linked to a disclosed gRNA can comprise a glutamine tRNA promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect, a disclosed promoter for the one or more disclosed guide RNA sequences can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced

In an aspect, a disclosed isolated nucleic acid molecule can comprise nucleic acid sequence encoding a Cas9 nuclease and a promoter operably linked to the Cas9. In an aspect, a disclosed Cas9 nuclease can comprise a SaCas9 nuclease. For example, in an aspect, a disclosed SaCas9 nuclease can comprise the sequence set forth in SEQ ID NO: 19 or a sequence having about 60%, about 65%, about 70%, about 75%, about 80%, about 85%, about 90%, about 95%, or more than 95% identity to the sequence set forth in SEQ ID NO: 19.

In an aspect, a disclosed Cas9 nuclease can create a double-strand break (DSB) at or near the IVS1 variant in the GAA locus that results in a permanent integration of the repair template, or a double-strand break (DSB) at or near the ΔT525 variant in Exon 2 of the GAA locus that results in a permanent integration of the repair template, a double-strand break (DSB) at or near the 1826DupA variant in Exon 13 of the GAA locus that results in a permanent integration of the repair template. In an aspect, a disclosed Cas9 nuclease can create a double-strand break (DSB) at or near any mutation in the GAA locus that results in a permanent integration of the repair template.

In an aspect, a disclosed promoter can drive expression of the Cas9 nuclease. In an aspect, a disclosed promoter that drives expression of the Cas9 nuclease can comprise a CMV promoter, a EF1a promoter, a chicken beta-actin promoter, a liver-specific promoter, or a G6PC promoter, or a minimal CMV promoter, a minimal EF1a promoter, a minimal chicken beta-actin promoter, a minimal liver-specific promoter, or a minimal G6PC promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect, a disclosed isolated nucleic acid molecule can comprise a nuclear localization signal (NLS). In an aspect, a disclosed NLS can comprise the sequence set forth in SEQ ID NO:50 or SEQ ID NO:51.

In an aspect, a disclosed isolated nucleic acid molecule can comprise one or more inverted terminal repeats (ITRs). In an aspect, the one or more disclosed ITRs can be derived from AAV2 or AAV9. In an aspect, a disclosed ITR can comprise the sequence set forth in any one of SEQ ID NO: 20, SEQ ID NO:21, or SEQ ID NO:22. In an aspect, a disclosed isolated nucleic acid molecule can comprise a polyA sequence. In an aspect, a disclosed polyA sequence can comprise the sequence set forth in SEQ ID NO:52 or SEQ ID NO:53. In an aspect, a disclosed isolated nucleic acid molecule can comprise one or more hemagglutinin (HA) tags. In an aspect, a disclosed HA tag can comprise the sequence set forth in SEQ ID NO:23 or SEQ ID NO:24. In an aspect, a disclosed isolated nucleic acid molecule can comprise a CRISPR/transactivating RNA (chRNA) sequence. In an aspect, a chRNA sequence can comprise the sequence set forth in SEQ ID NO: 25.

In an aspect, a disclosed isolated nucleic acid molecule can repair a defective GAA gene. In an aspect, a disclosed isolated nucleic acid molecule can restore one or more aspects of cellular homeostasis and/or cellular functionality and/or metabolic dysregulation. In an aspect, a disclosed isolated nucleic acid molecule can improve the efficiency of gene editing. In an aspect, a disclosed isolated nucleic acid molecule can be used to stably integrate a transgene (such as, for example, GAA) into one or more disclosed cells. In an aspect, a disclosed isolated nucleic acid molecule can be used in a method of treating a Pompe patient or in a method of validating a gene editing system.

In an aspect, a disclosed isolated nucleic acid molecule can restore one or more aspects of cellular homeostasis and/or cellular functionality and/or metabolic dysregulation (such as, for example, glycogen accumulation). In an aspect, a disclosed isolated nucleic acid molecule can restore the functionality and/or structural integrity of a missing, deficient, and/or mutant protein or enzyme (such as, for example, a mutant GAA).

In an aspect, a disclosed nucleic acid sequence can have a coding sequence that is less than about 4.5 kilobases.

Disclosed herein is an isolated nucleic acid molecule for repairing the IVS1 variant of Pompe disease, comprising a nucleic acid sequence encoding a repair template, wherein the repair template comprises a first sequence having homology to a sequence in intron 1 of GAA and a second sequence having homology to a sequence in exon 2 of GAA, wherein the homologous sequences flank a promoter; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is an isolated nucleic acid molecule for repairing the ΔT525 variant of Pompe disease, comprising a nucleic acid sequence encoding a repair template, wherein the repair template comprises a sequence having homology to a sequence in exon 2 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is an isolated nucleic acid molecule for repairing the 1826DupA variant of Pompe disease, comprising a nucleic acid sequence encoding a repair template, wherein the repair template comprises a sequence having homology to a sequence in exon 13 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

In an aspect, a disclosed isolated nucleic molecule can comprise one or more HA tags, one or more ITRs, a polyA sequence, a nuclear location signal, or any combination thereof.

In an aspect, a disclosed repair template comprises about 300 bp to about 700 bp. In an aspect, a disclosed repair template can comprise about 400 bp, or about 600 bp, or about 650 bp.

In an aspect, a disclosed repair template can comprise the sequence set forth in SEQ ID NO: 26, SEQ ID NO:27, and SEQ ID NO:28, or a sequence having about 50%, about 60%, about 70% about 80%, about 90%, about 95%, or more identity to the sequence set forth in SEQ ID NO: 26, SEQ ID NO:27, and SEQ ID NO:28. In an aspect, a disclosed repair template can comprise a nucleic acid sequence encoding wild-type GAA or a portion thereof. In an aspect, a disclosed GAA can comprise the sequence set forth in SEQ ID NO:62, or a sequence having about 50%, about 60%, about 70% about 80%, about 90%, about 95%, or more identity to the sequence set forth in SEQ ID NO:62.

In an aspect, a disclosed repair template can comprise a restriction site. Restriction enzymes are known to the art.

For example, a disclosed repair template can comprise a first sequence having homology to a sequence in intron 1 of GAA and a second sequence having homology to a sequence in exon 2 of GAA. In an aspect, a disclosed first homologous sequence can comprise the sequence of a wild-type GAA or a portion thereof, and a disclosed second homologous sequence can comprise the sequence of a wild-type GAA or a portion thereof, or any combination thereof. In an aspect, a disclosed GAA can comprise the sequence set forth in SEQ ID NO:62.

In an aspect, a disclosed repair template can comprise a promoter sequence between the first homologous sequence and the second homology sequence. In an aspect, a disclosed promoter can comprise a CMV promoter, a EF1a promoter, a chicken beta-actin promoter, a liver-specific promoter, or a G6PC promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect, a disclosed first homologous sequence can comprise about 175 bp to about 225 bp or about 200 bp to about 220 bp. For example, in an aspect, a disclosed first homologous sequence can comprise the sequence set forth in SEQ ID NO:54, SEQ ID NO:55, SEQ ID NO:56, or SEQ ID NO:57. In an aspect, a disclosed second homologous sequence can comprise about 175 bp to about 225 bp or about 200 bp to about 220 bp. In an aspect, a disclosed second homologous can comprise the sequence set forth in SEQ ID NO:58, SEQ ID NO:59, SEQ ID NO: 60, or SEQ ID NO:61. In an aspect, a disclosed first homologous sequence can comprise the sequence of a wild-type GAA or a portion thereof, a disclosed second homologous sequence can comprise the sequence of a wild-type GAA or a portion thereof. In an aspect, a disclosed GAA can comprise the sequence set forth in SEQ ID NO:62.

In an aspect, a disclosed repair template can comprise a sequence having homology to a sequence in exon 2 of GAA. In an aspect, a disclosed repair template can comprise a sequence having homology to a sequence in exon 13 of GAA. (m1826DupA)

In an aspect, a disclosed isolated nucleic acid molecule can comprise a nucleic acid sequence encoding a promoter that is operably linked to a nucleic acid sequence encoding a guide RNA (gRNA) and drives the expression of the gRNA. In an aspect, a disclosed gRNA can target a PAM sequence at or near the IVS1 variant near the boundary between intron 1 and exon 2 in the GAA gene, a PAM sequence at or near the ΔT525 variant in Exon 2 in the GAA gene, or a PAM sequence at or near the m 1826DupA variant in Exon 13 in the GAA gene. In an aspect, a disclosed gRNA can target a PAM sequence at or near any mutation in the GAA gene.

In an aspect, a disclosed gRNA can comprise the sequence set forth in any of SEQ ID NO: 29-SEQ ID NO:36.

In an aspect, a disclosed promoter operably linked to a disclosed gRNA can comprise a RNA polymerase III (Poly III) promoter. In an aspect, a disclosed Poly III promoter can comprise a U3 promoter, a U6 promoter, or a H1 promoter. In an aspect, a disclosed promoter operably linked to a disclosed gRNA can comprise a glutamine tRNA promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect, a disclosed promoter for the one or more disclosed guide RNA sequences can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced

In an aspect, a disclosed isolated nucleic acid molecule can comprise nucleic acid sequence encoding a Cas9 nuclease and a promoter operably linked to the Cas9. In an aspect, a disclosed Cas9 nuclease can comprise a SaCas9 nuclease. For example, in an aspect, a disclosed SaCas9 nuclease can comprise the sequence set forth in SEQ ID NO: 19 or a sequence having about 60%, about 65%, about 70%, about 75%, about 80%, about 85%, about 90%, about 95%, or more than 95% identity to the sequence set forth in SEQ ID NO: 19.

In an aspect, a disclosed Cas9 nuclease can create a double-strand break (DSB) at or near the IVS1 variant in the GAA locus that results in a permanent integration of the repair template, or a double-strand break (DSB) at or near the ΔT525 variant in Exon 2 of the GAA locus that results in a permanent integration of the repair template, a double-strand break (DSB) at or near the 1826DupA variant in Exon 13 of the GAA locus that results in a permanent integration of the repair template. In an aspect, a disclosed Cas9 nuclease can create a double-strand break (DSB) at or near any mutation in the GAA locus that results in a permanent integration of the repair template.

In an aspect, a disclosed promoter can drive expression of the Cas9 nuclease. In an aspect, a disclosed promoter that drives expression of the Cas9 nuclease can comprise a CMV promoter, a EF1a promoter, a chicken beta-actin promoter, a liver-specific promoter, or a G6PC promoter, or a minimal CMV promoter, a minimal EF1a promoter, a minimal chicken beta-actin promoter, a minimal liver-specific promoter, or a minimal G6PC promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect, a disclosed isolated nucleic acid molecule can comprise a nuclear localization signal (NLS). In an aspect, a disclosed NLS can comprise the sequence set forth in SEQ ID NO:50 or SEQ ID NO:51. In an aspect, a disclosed isolated nucleic acid molecule can comprise one or more inverted terminal repeats (ITRs). In an aspect, the one or more disclosed ITRs can be derived from AAV2 or AAV9. In an aspect, a disclosed ITR can comprise the sequence set forth in any one of SEQ ID NO:20, SEQ ID NO:21, or SEQ ID NO:22. In an aspect, a disclosed isolated nucleic acid molecule can comprise a polyA sequence. In an aspect, a disclosed polyA sequence can comprise the sequence set forth in SEQ ID NO:52 or SEQ ID NO:53. In an aspect, a disclosed isolated nucleic acid molecule can comprise one or more hemagglutinin (HA) tags. In an aspect, a disclosed HA tag can comprise the sequence set forth in SEQ ID NO:23 or SEQ ID NO:24. In an aspect, a disclosed isolated nucleic acid molecule can comprise a CRISPR/transactivating RNA (chRNA) sequence. In an aspect, a chRNA sequence can comprise the sequence set forth in SEQ ID NO: 25.

In an aspect, a disclosed isolated nucleic acid molecule can restore one or more aspects of cellular homeostasis and/or cellular functionality and/or metabolic dysregulation. In an aspect, a disclosed isolated nucleic acid molecule can improve the efficiency of gene editing. In an aspect, a disclosed isolated nucleic acid molecule can be used to stably integrate a transgene (such as, for example, GAA) into one or more disclosed cells. In an aspect, a disclosed isolated nucleic acid molecule can be used in a method of treating a Pompe patient or in a method of validating a gene editing system.

In an aspect, a disclosed isolated nucleic acid molecule can restore one or more aspects of cellular homeostasis and/or cellular functionality and/or metabolic dysregulation (such as, for example, glycogen accumulation). In an aspect, a disclosed isolated nucleic acid molecule can restore the functionality and/or structural integrity of a missing, deficient, and/or mutant protein or enzyme (such as, for example, a mutant GAA).

In an aspect, a disclosed nucleic acid sequence to be trans-spliced can be CpG depleted and codon-optimized for expression in a human cell. In an aspect, “CpG-free” can mean completely free of CpGs or partially free of CpGs. In an aspect, “CpG-free” can mean “CpG-depleted”. In an aspect, “CpG-depleted” can mean “CpG-free”. In an aspect, “CpG-depleted” can mean completely depleted of CpGs or partially depleted of CpGs. In an aspect, “CpG-free” can mean “CpG-optimized” for a desired and/or ideal expression level. CpG depletion and/or optimization is known to the skilled person in the art.

Disclosed herein is an expression cassette, comprising a nucleic acid sequence encoding a repair template for Pompe disease.

Disclosed herein is an expression cassette for repairing the IVS1 variant of Pompe disease, comprising a nucleic acid sequence encoding a repair template, wherein the repair template comprises a first sequence having homology to a sequence in intron 1 of GAA and a second sequence having homology to a sequence in exon 2 of GAA, wherein the homologous sequences flank a promoter; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is an expression cassette for repairing the ΔT525 variant of Pompe disease, comprising a nucleic acid sequence encoding a repair template, wherein the repair template comprises a sequence having homology to a sequence in exon 2 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is an expression cassette for repairing the 1826DupA variant of Pompe disease, comprising a nucleic acid sequence encoding a repair template, wherein the repair template comprises a sequence having homology to a sequence in exon 13 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is an isolated donor nucleic acid molecule, comprising a nucleic acid sequence encoding a repair template, a promoter operably linked to the repair template, and a poly A sequence.

In an aspect of a disclosed isolated donor nucleic acid molecule, a disclosed repair template is for the IVS1 variant of Pompe disease, for the ΔT525 variant of Pompe disease, or for the 1826DupA variant of Pompe disease. In an aspect of a disclosed isolated donor nucleic acid molecule, a disclosed repair template is for any mutation in the gene encoding alpha glucosidase (GAA). In an aspect, a disclosed repair template is for one or more mutations in the gene encoding alpha glucosidase (GAA).

In an aspect of a disclosed isolated donor nucleic acid molecule, a disclosed repair template comprises about 300 bp to about 700 bp. In an aspect, a disclosed repair template comprises about 400 bp, about 600 bp, or about 650 bp.

In an aspect, a disclosed repair template can comprise the sequence set forth in SEQ ID NO: 26, SEQ ID NO:27, and SEQ ID NO:28, or a sequence having about 50%, about 60%, about 70% about 80%, about 90%, about 95%, or more identity to the sequence set forth in SEQ ID NO: 26, SEQ ID NO:27, and SEQ ID NO:28. In an aspect, a disclosed repair template can comprise a nucleic acid sequence encoding wild-type GAA or a portion thereof. In an aspect, a disclosed GAA can comprise the sequence set forth in SEQ ID NO:62, or a sequence having about 50%, about 60%, about 70% about 80%, about 90%, about 95%, or more identity to the sequence set forth in SEQ ID NO:62.

In an aspect of a disclosed isolated donor nucleic acid molecule, a disclosed repair template can comprise restriction site. Restriction sites are known to the art.

In an aspect of a disclosed isolated donor nucleic acid molecule, a disclosed repair template can comprise a first sequence having homology to a sequence in intron 1 of GAA and a second sequence having homology to a sequence in exon 2 of GAA. In an aspect of a disclosed isolated donor nucleic acid molecule, a disclosed repair template can comprise a sequence having homology to a sequence in exon 2 of GAA or can comprise a sequence having homology to a sequence in exon 13 of GAA.

In an aspect of a disclosed isolated donor nucleic acid molecule, a disclosed repair template can comprise a promoter sequence between the first homologous sequence and the second homology sequence.

In an aspect, a disclosed first homologous sequence can comprise about 175 bp to about 225 bp or about 200 bp to about 220 bp. For example, in an aspect, a disclosed first homologous sequence can comprise the sequence set forth in SEQ ID NO:54, SEQ ID NO:55, SEQ ID NO:56, or SEQ ID NO:57. In an aspect, a disclosed second homologous sequence can comprise about 175 bp to about 225 bp or about 200 bp to about 220 bp. In an aspect, a disclosed second homologous can comprise the sequence set forth in SEQ ID NO:58, SEQ ID NO:59, SEQ ID NO: 60, or SEQ ID NO:61.

In an aspect, a disclosed first homologous sequence can comprise the sequence of a wild-type GAA or a portion thereof. In an aspect, a disclosed GAA can comprise the sequence set forth in SEQ ID NO:62. In an aspect, a disclosed second homologous sequence can comprise the sequence of a wild-type GAA or a portion thereof. In an aspect, a disclosed GAA can comprise the sequence set forth in SEQ ID NO:62.

In an aspect, a disclosed isolated donor nucleic acid molecule can comprise one or more inverted terminal repeats (ITRs). In an aspect, a disclosed ITR can be derived from AAV2 or AAV9. In an aspect, a disclosed ITR can comprise the sequence set forth in any one of SEQ ID NO: 20, SEQ ID NO:21, or SEQ ID NO:22. In an aspect, a disclosed isolated donor nucleic acid molecule can comprise a polyA sequence. In an aspect, a disclosed polyA sequence can comprise the sequence set forth in SEQ ID NO:52 or SEQ ID NO:53.

Disclosed herein is an isolated CRISPR nucleic acid molecule comprising a nucleic acid sequence encoding a guide RNA (gRNA) operably linked to a first promoter and a nucleic acid sequence encoding a Cas9 nuclease operably linked to second promoter.

In an aspect of a disclosed isolated CRISPR nucleic acid molecule, a disclosed gRNA can target a PAM sequence at or near the IVS1 variant near the boundary between intron 1 and exon 2 in the GAA gene. In an aspect, a disclosed gRNA can target a PAM sequence at or near the ΔT525 variant in Exon 2 in the GAA gene. In an aspect, a disclosed gRNA can comprise the sequence set forth in any of SEQ ID NO:29-SEQ ID NO:36.

In an aspect of a disclosed isolated CRISPR nucleic acid molecule, a disclosed first promoter operably linked to the gRNA can comprise a RNA polymerase III (Poly III) promoter. In an aspect, a disclosed Poly III promoter can comprise a U3 promoter, a U6 promoter, or a H1 promoter. In an aspect, a disclosed first promoter operably linked to the gRNA can comprise a glutamine tRNA promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect of a disclosed isolated CRISPR nucleic acid molecule, a disclosed Cas9 nuclease can comprise a SaCas9 nuclease. In an aspect, the sequence of a disclosed SaCas9 nuclease can comprise the sequence set forth in SEQ ID NO: 19 or a sequence having about 60%, about 65%, about 70%, about 75%, about 80%, about 85%, about 90%, about 95%, or more than 95% identity to the sequence set forth in SEQ ID NO:19.

In an aspect of a disclosed isolated CRISPR nucleic acid molecule, a disclosed second promoter operably linked to the Cas9 nuclease can comprise a CMV promoter, a EF1a promoter, a chicken beta-actin promoter, a liver-specific promoter, or a G6PC promoter. In an aspect, a disclosed promoter can comprise the sequence set forth in any one of SEQ ID NO:40-SEQ ID NO: 48, or a sequence having about 50%, about 60%, about 70% about 80%, about 90%, about 95%, or more identity to the sequence set forth in any one of SEQ ID NO:40-SEQ ID NO:48.

In an aspect, a disclosed isolated CRISPR nucleic acid molecule can comprise a nuclear localization signal (NLS). In an aspect, a disclosed NLS can comprise the sequence set forth in SEQ ID NO:50 or SEQ ID NO:51. In an aspect, a disclosed isolated CRISPR nucleic acid molecule can comprise one or more inverted terminal repeats (ITRs). In the aspect, a disclosed ITRs can be derived from AAV2 or AAV9. In an aspect, a disclosed ITR can comprise the sequence set forth in any one of SEQ ID NO:20, SEQ ID NO:21, or SEQ ID NO:22. In an aspect, a disclosed isolated CRISPR nucleic acid molecule can comprise a polyA sequence. In an aspect, a disclosed polyA sequence can comprise the sequence set forth in SEQ ID NO:52 or SEQ ID NO: 53. In an aspect, a disclosed isolated CRISPR nucleic acid molecule can comprise one or more hemagglutinin (HA) tags. In an aspect, a disclosed HA tag can comprise the sequence set forth in SEQ ID NO:23 or SEQ ID NO:24. In an aspect, a disclosed isolated nucleic acid molecule can comprise a CRISPR/transactivating RNA (chRNA) sequence. In an aspect, a chRNA sequence can comprise the sequence set forth in SEQ ID NO:25.

### 2. Gene Editing Systems

Disclosed herein is a gene editing system for stably integrating an alpha-glucosidase transgene into one or more cells, comprising a first vector comprising an isolated donor nucleic acid molecule; and a second vector comprising an isolated CRISPR nucleic acid molecule.

In an aspect, a disclosed donor nucleic acid molecule can comprise a nucleic acid sequence encoding a repair template, a promoter operably linked to the repair template, and a poly A sequence; and a disclosed CRISPR nucleic acid molecule can comprise a nucleic acid sequence encoding a guide RNA (gRNA) operably linked to a first promoter and a nucleic acid sequence encoding a Cas9 nuclease operably linked to second promoter.

In an aspect, a disclosed repair template can be for the IVS1 variant of Pompe disease, for the ΔT525 variant of Pompe disease, or for the 1826DupA variant of Pompe disease. In an aspect, a disclosed repair template can be for any mutation in the gene encoding alpha glucosidase (GAA). In an aspect, a disclosed repair template can be for any mutation in the gene encoding alpha glucosidase (GAA) or can be for one or more mutations in the gene encoding alpha glucosidase (GAA).

In an aspect, a disclosed repair template comprises about 300 bp to about 700 bp. In an aspect, a disclosed repair template can comprise about 400 bp, or about 600 bp, or about 650 bp.

In an aspect, a disclosed repair template can comprise the sequence set forth in SEQ ID NO: 26, SEQ ID NO:27, and SEQ ID NO:28, or a sequence having about 50%, about 60%, about 70% about 80%, about 90%, about 95%, or more identity to the sequence set forth in SEQ ID NO: 26, SEQ ID NO:27, and SEQ ID NO:28. In an aspect, a disclosed repair template can comprise a nucleic acid sequence encoding wild-type GAA or a portion thereof. In an aspect, a disclosed GAA can comprise the sequence set forth in SEQ ID NO:62, or a sequence having about 50%, about 60%, about 70% about 80%, about 90%, about 95%, or more identity to the sequence set forth in SEQ ID NO:62.

In an aspect, a disclosed repair template can comprise a restriction site. Restriction enzymes are known to the art.

For example, a disclosed repair template can comprise a first sequence having homology to a sequence in intron 1 of GAA and a second sequence having homology to a sequence in exon 2 of GAA. In an aspect, a disclosed first homologous sequence can comprise the sequence of a wild-type GAA or a portion thereof, and a disclosed second homologous sequence can comprise the sequence of a wild-type GAA or a portion thereof, or any combination thereof. In an aspect, a disclosed GAA can comprise the sequence set forth in SEQ ID NO:62.

In an aspect, a disclosed repair template can comprise a promoter sequence between the first homologous sequence and the second homology sequence. In an aspect, a disclosed promoter can comprise a CMV promoter, a EF1a promoter, a chicken beta-actin promoter, a liver-specific promoter, or a G6PC promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect, a disclosed first homologous sequence can comprise about 175 bp to about 225 bp or about 200 bp to about 220 bp. For example, in an aspect, a disclosed first homologous sequence can comprise the sequence set forth in SEQ ID NO:54, SEQ ID NO:55, SEQ ID NO:56, or SEQ ID NO:57. In an aspect, a disclosed second homologous sequence can comprise about 175 bp to about 225 bp or about 200 bp to about 220 bp. In an aspect, a disclosed second homologous can comprise the sequence set forth in SEQ ID NO:58, SEQ ID NO:59, SEQ ID NO: 60, or SEQ ID NO:61. In an aspect, a disclosed first homologous sequence can comprise the sequence of a wild-type GAA or a portion thereof, a disclosed second homologous sequence can comprise the sequence of a wild-type GAA or a portion thereof. In an aspect, a disclosed GAA can comprise the sequence set forth in SEQ ID NO:62. In an aspect, a disclosed repair template can comprise a sequence having homology to a sequence in exon 2 of GAA. In an aspect, a disclosed repair template can comprise a sequence having homology to a sequence in exon 13 of GAA.

In an aspect of a disclosed gene editing system, a disclosed CRISPR isolated nucleic acid molecule can comprise a nucleic acid sequence encoding a promoter that is operably linked to a nucleic acid sequence encoding a guide RNA (gRNA) and drives the expression of the gRNA. In an aspect, a disclosed gRNA can target a PAM sequence at or near the IVS1 variant near the boundary between intron 1 and exon 2 in the GAA gene, a PAM sequence at or near the ΔT525 variant in Exon 2 in the GAA gene, or a PAM sequence at or near the m1826DupA variant in Exon 13 in the GAA gene. In an aspect, a disclosed gRNA can target a PAM sequence at or near any mutation in the GAA gene.

In an aspect, a disclosed gRNA can comprise the sequence set forth in any of SEQ ID NO: 29-SEQ ID NO:36.

In an aspect, a disclosed promoter operably linked to a disclosed gRNA can comprise a RNA polymerase III (Poly III) promoter. In an aspect, a disclosed Poly III promoter can comprise a U3 promoter, a U6 promoter, or a H1 promoter. In an aspect, a disclosed promoter operably linked to a disclosed gRNA can comprise a glutamine tRNA promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect of a disclosed gene editing system, a disclosed CRISPR isolated nucleic acid molecule can comprise nucleic acid sequence encoding a Cas9 nuclease and a promoter operably linked to the Cas9. In an aspect, a disclosed Cas9 nuclease can comprise a SaCas9 nuclease. For example, in an aspect, a disclosed SaCas9 nuclease can comprise the sequence set forth in SEQ ID NO: 19 or a sequence having about 60%, about 65%, about 70%, about 75%, about 80%, about 85%, about 90%, about 95%, or more than 95% identity to the sequence set forth in SEQ ID NO: 19.

In an aspect, a disclosed Cas9 nuclease can create a double-strand break (DSB) at or near the IVS1 variant in the GAA locus that results in a permanent integration of the repair template, or a double-strand break (DSB) at or near the ΔT525 variant in Exon 2 of the GAA locus that results in a permanent integration of the repair template, a double-strand break (DSB) at or near the 1826DupA variant in Exon 13 of the GAA locus that results in a permanent integration of the repair template. In an aspect, a disclosed Cas9 nuclease can create a double-strand break (DSB) at or near any mutation in the GAA locus that results in a permanent integration of the repair template.

In an aspect, a disclosed promoter operably linked the Cas9 nuclease can drive expression of the Cas9 nuclease. In an aspect, a disclosed promoter that drives expression of the Cas9 nuclease can comprise a CMV promoter, a EF1a promoter, a chicken beta-actin promoter, a liver-specific promoter, or a G6PC promoter, or a minimal CMV promoter, a minimal EF1a promoter, a minimal chicken beta-actin promoter, a minimal liver-specific promoter, or a minimal G6PC promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO: 40-SEQ ID NO:48.

In an aspect of a disclosed gene editing system, a disclosed CRISPR nucleic acid molecule can comprise a nuclear localization signal (NLS). In an aspect, a disclosed NLS can comprise the sequence set forth in SEQ ID NO:50 or SEQ ID NO:51. In an aspect of a disclosed gene editing system, a disclosed CRISPR nucleic acid molecule can comprise one or more inverted terminal repeats (ITRs). In an aspect, a disclosed ITR can be derived from AAV2 or AAV9. In an aspect, a disclosed ITR can comprise the sequence set forth in any one of SEQ ID NO:20, SEQ ID NO:21, or SEQ ID NO:22. In an aspect of a disclosed gene editing system, a disclosed CRISPR nucleic acid molecule can comprise a polyA sequence. In an aspect, a disclosed polyA sequence can comprise the sequence set forth in SEQ ID NO:52 or SEQ ID NO:53. In an aspect of a disclosed gene editing system, a disclosed CRISPR nucleic acid molecule can comprise one or more hemagglutinin (HA) tags. In an aspect, a disclosed HA tag can comprise the sequence set forth in SEQ ID NO:23 or SEQ ID NO:24. In an aspect, a disclosed isolated nucleic acid molecule can comprise a CRISPR/transactivating RNA (chRNA) sequence. In an aspect, a chRNA sequence can comprise the sequence set forth in SEQ ID NO:25.

In an aspect, a disclosed gene editing system can restore one or more aspects of cellular homeostasis and/or cellular functionality and/or metabolic dysregulation. In an aspect, a disclosed gene editing system can improve the efficiency of gene editing. In an aspect, a disclosed gene editing system can be used to stably integrate a transgene (such as, for example, GAA) into one or more disclosed cells. In an aspect, a disclosed gene editing system can be used in a method of treating a Pompe patient or in a method of validating a gene editing system.

In an aspect, a disclosed gene editing system can restore one or more aspects of cellular homeostasis and/or cellular functionality and/or metabolic dysregulation (such as, for example, glycogen accumulation). In an aspect, a disclosed gene editing system can restore the functionality and/or structural integrity of a missing, deficient, and/or mutant protein or enzyme (such as, for example, a mutant GAA).

### 3. Vectors

Disclosed herein is a vector comprising a disclosed isolated nucleic acid molecule. Disclosed herein is a vector comprising a disclosed isolated donor nucleic acid molecule. Disclosed herein is a vector comprising a disclosed isolated CRISPR nucleic acid molecule. Disclosed herein is a vector comprising an isolated nucleic acid molecule comprising a nucleic acid sequence encoding a repair template for any mutation in the alpha glucosidase (GAA) gene.

Disclosed herein is a vector comprising an isolated nucleic acid molecule for repairing the IVS1 variant of Pompe disease, comprising a nucleic acid sequence encoding a repair template, wherein the repair template comprises a first sequence having homology to a sequence in intron 1 of GAA and a second sequence having homology to a sequence in exon 2 of GAA, wherein the homologous sequences flank a promoter; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is a vector comprising an isolated nucleic acid molecule for repairing the ΔT525 variant of Pompe disease, comprising a nucleic acid sequence encoding a repair template, wherein the repair template comprises a sequence having homology to a sequence in exon 2 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is a vector comprising an isolated nucleic acid molecule for repairing the 1826DupA variant of Pompe disease, comprising a nucleic acid sequence encoding a repair template, wherein the repair template comprises a sequence having homology to a sequence in exon 13 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is a vector comprising an isolated nucleic acid molecule comprising a nucleic acid sequence encoding a repair template for Pompe disease.

Disclosed herein is a vector comprising an isolated nucleic acid molecule comprising a nucleic acid sequence encoding a repair template is for the IVS1 variant of Pompe disease, for the ΔT525 variant of Pompe disease, or for the 1826DupA variant of Pompe disease.

In an aspect of a disclosed vector, a disclosed repair template comprises about 300 bp to about 700 bp. In an aspect, a disclosed repair template can comprise about 400 bp, or about 600 bp, or about 650 bp.

In an aspect, a disclosed repair template can comprise the sequence set forth in SEQ ID NO: 26, SEQ ID NO:27, and SEQ ID NO:28, or a sequence having about 50%, about 60%, about 70% about 80%, about 90%, about 95%, or more identity to the sequence set forth in SEQ ID NO: 26, SEQ ID NO:27, and SEQ ID NO:28. In an aspect, a disclosed repair template can comprise a nucleic acid sequence encoding wild-type GAA or a portion thereof. In an aspect, a disclosed GAA can comprise the sequence set forth in SEQ ID NO:62, or a sequence having about 50%, about 60%, about 70% about 80%, about 90%, about 95%, or more identity to the sequence set forth in SEQ ID NO:62.

In an aspect, a disclosed repair template can comprise a restriction site. Restriction enzymes are known to the art.

In an aspect of a disclosed vector, a disclosed repair template can comprise a nucleic acid sequence encoding wild-type GAA or a portion thereof. For example, a disclosed repair template can comprise a first sequence having homology to a sequence in intron 1 of GAA and a second sequence having homology to a sequence in exon 2 of GAA. In an aspect, a disclosed first homologous sequence can comprise the sequence of a wild-type GAA or a portion thereof, and a disclosed second homologous sequence can comprise the sequence of a wild-type GAA or a portion thereof, or any combination thereof. In an aspect, a disclosed GAA can comprise the sequence set forth in SEQ ID NO:62.

In an aspect, a disclosed repair template can comprise a promoter sequence between the first homologous sequence and the second homology sequence. In an aspect, a disclosed promoter can comprise a CMV promoter, a EF1a promoter, a chicken beta-actin promoter, a liver-specific promoter, or a G6PC promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect, a disclosed first homologous sequence can comprise about 175 bp to about 225 bp or about 200 bp to about 220 bp. For example, in an aspect, a disclosed first homologous sequence can comprise the sequence set forth in SEQ ID NO:54, SEQ ID NO:55, SEQ ID NO:56, or SEQ ID NO:57. In an aspect, a disclosed second homologous sequence can comprise about 175 bp to about 225 bp or about 200 bp to about 220 bp. In an aspect, a disclosed second homologous can comprise the sequence set forth in SEQ ID NO:58, SEQ ID NO:59, SEQ ID NO: 60, or SEQ ID NO:61. In an aspect, a disclosed first homologous sequence can comprise the sequence of a wild-type GAA or a portion thereof, a disclosed second homologous sequence can comprise the sequence of a wild-type GAA or a portion thereof. In an aspect, a disclosed GAA can comprise the sequence set forth in SEQ ID NO:62.

In an aspect, a disclosed repair template can comprise a sequence having homology to a sequence in exon 2 of GAA. In an aspect, a disclosed repair template can comprise a sequence having homology to a sequence in exon 13 of GAA.

In an aspect of a disclosed vector, a disclosed isolated nucleic acid molecule can comprise a nucleic acid sequence encoding a promoter that is operably linked to a nucleic acid sequence encoding a guide RNA (gRNA) and drives the expression of the gRNA. In an aspect, a disclosed gRNA can target a PAM sequence at or near the IVS1 variant near the boundary between intron 1 and exon 2 in the GAA gene, a PAM sequence at or near the ΔT525 variant in Exon 2 in the GAA gene, or a PAM sequence at or near the m1826DupA variant in Exon 13 in the GAA gene. In an aspect, a disclosed gRNA can target a PAM sequence at or near any mutation in the GAA gene.

In an aspect, a disclosed gRNA can comprise the sequence set forth in any of SEQ ID NO: 29-SEQ ID NO:36.

In an aspect, a disclosed promoter operably linked to a disclosed gRNA can comprise a RNA polymerase III (Poly III) promoter. In an aspect, a disclosed Poly III promoter can comprise a U3 promoter, a U6 promoter, or a H1 promoter. In an aspect, a disclosed promoter operably linked to a disclosed gRNA can comprise a glutamine tRNA promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect, a disclosed promoter for the one or more disclosed guide RNA sequences can be tissue-specific or ubiquitous and can be constitutive or inducible, depending on the pattern of the gene expression desired. A promoter can be native or foreign and can be a natural or a synthetic sequence. By foreign, it is intended that the transcriptional initiation region is not found in the wild-type host into which the transcriptional initiation region is introduced

In an aspect, a disclosed isolated nucleic acid molecule can comprise nucleic acid sequence encoding a Cas9 nuclease and a promoter operably linked to the Cas9. In an aspect, a disclosed Cas9 nuclease can comprise a SaCas9 nuclease. For example, in an aspect, a disclosed SaCas9 nuclease can comprise the sequence set forth in SEQ ID NO: 19 or a sequence having about 60%, about 65%, about 70%, about 75%, about 80%, about 85%, about 90%, about 95%, or more than 95% identity to the sequence set forth in SEQ ID NO: 19.

In an aspect, a disclosed Cas9 nuclease can create a double-strand break (DSB) at or near the IVS1 variant in the GAA locus that results in a permanent integration of the repair template, or a double-strand break (DSB) at or near the ΔT525 variant in Exon 2 of the GAA locus that results in a permanent integration of the repair template, a double-strand break (DSB) at or near the 1826DupA variant in Exon 13 of the GAA locus that results in a permanent integration of the repair template. In an aspect of a disclosed vector, a disclosed Cas9 nuclease can create a double-strand break (DSB) at or near any mutation in the GAA locus that results in a permanent integration of the repair template.

In an aspect of a disclosed vector, a disclosed promoter can drive expression of the Cas9 nuclease. In an aspect, a disclosed promoter that drives expression of the Cas9 nuclease can comprise a CMV promoter, a EF1a promoter, a chicken beta-actin promoter, a liver-specific promoter, or a G6PC promoter, or a minimal CMV promoter, a minimal EF1a promoter, a minimal chicken beta-actin promoter, a minimal liver-specific promoter, or a minimal G6PC promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO: 40-SEQ ID NO:48.

In an aspect of a disclosed vector, a disclosed isolated nucleic acid molecule can comprise a nuclear localization signal (NLS). In an aspect, a disclosed NLS can comprise the sequence set forth in SEQ ID NO:50 or SEQ ID NO:51. In an aspect of a disclosed vector, a disclosed isolated nucleic acid molecule can comprise one or more inverted terminal repeats (ITRs). In an aspect, the one or more disclosed ITRs can be derived from AAV2 or AAV9. In an aspect, a disclosed ITR can comprise the sequence set forth in any one of SEQ ID NO:20, SEQ ID NO:21, or SEQ ID NO: 22. In an aspect of a disclosed vector, a disclosed isolated nucleic acid molecule can comprise a polyA sequence. In an aspect, a disclosed poly A sequence can comprise the sequence set forth in SEQ ID NO:52 or SEQ ID NO:53. In an aspect of a disclosed vector, a disclosed isolated nucleic acid molecule can comprise one or more hemagglutinin (HA) tags. In an aspect, a disclosed HA tag can comprise the sequence set forth in SEQ ID NO:23 or SEQ ID NO:24. In an aspect, a disclosed isolated nucleic acid molecule can comprise a CRISPR/transactivating RNA (chRNA) sequence. In an aspect, a chRNA sequence can comprise the sequence set forth in SEQ ID NO: 25.

In an aspect, a disclosed vector can repair a defective GAA gene. In an aspect, a disclosed vector can restore one or more aspects of cellular homeostasis and/or cellular functionality and/or metabolic dysregulation. In an aspect, a disclosed vector can improve the efficiency of gene editing. In an aspect, a disclosed vector can be used to stably integrate a transgene (such as, for example, GAA) into one or more disclosed cells. In an aspect, a disclosed vector can be used in a method of treating a Pompe patient or in a method of validating a gene editing system.

In an aspect, a disclosed vector can restore one or more aspects of cellular homeostasis and/or cellular functionality and/or metabolic dysregulation (such as, for example, glycogen accumulation). In an aspect, a disclosed vector can restore the functionality and/or structural integrity of a missing, deficient, and/or mutant protein or enzyme (such as, for example, a mutant GAA).

In an aspect of a disclosed vector, a disclosed nucleic acid sequence can have a coding sequence that is less than about 4.5 kilobases.

In an aspect, a therapeutically effective amount of disclosed vector can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results. In an aspect, a therapeutically effective amount of disclosed vector can comprise a range determined by a skilled person.

In an aspect, a disclosed vector can repair a defective GAA gene. In an aspect, a disclosed vector can restore one or more aspects of cellular homeostasis and/or cellular functionality and/or metabolic dysregulation. In an aspect, a disclosed vector can improve the efficiency of gene editing. In an aspect, a disclosed vector can restore one or more aspects of cellular homeostasis and/or cellular functionality and/or metabolic dysregulation (such as, for example, glycogen accumulation). In an aspect, a disclosed vector can restore the functionality and/or structural integrity of a missing, deficient, and/or mutant protein or enzyme (such as, for example, a mutant GAA).

Disclosed herein is a vector comprising the sequence set forth in any one of SEQ ID NO:01-SEQ ID NO:18 or any portion thereof.

In an aspect, a disclosed vector can be a viral vector or a non-viral vector. In an aspect, a disclosed non-viral vector can be a polymer-based vector, a peptide-based vector, a lipid nanoparticle, a solid lipid nanoparticle, or a cationic lipid-based vector. In an aspect, a disclosed vector can comprise exosomes, extracellular vesicles, and virus like particles. In an aspect, a disclosed viral vector can be an adenovirus vector, an AAV vector, a herpes simplex virus vector, a retrovirus vector, a lentivirus vector, and alphavirus vector, a Flavivirus vector, a rhabdovirus vector, a measles virus vector, a Newcastle disease viral vector, a poxvirus vector, or a picornavirus.

In an aspect, a disclosed viral vector can be an adeno-associated virus (AAV) vector In an aspect, a disclosed AAV vector can include naturally isolated serotypes including, but not limited to, AAV1, AAV2, AAV3 (including 3a and 3b), AAV4, AAV5, AAV6, AAV7, AAV8, AAVrh8, AAV9, AAV10, AAVrh10, AAV11, AAV12, AAV13, AAVrh39, AAVrh43, AAVcy.7 as well as bovine AAV, caprine AAV, canine AAV, equine AAV, ovine AAV, avian AAV, primate AAV, non-primate AAV, and any other virus classified by the International Committee on Taxonomy of Viruses (ICTV) as an AAV. In an aspect, an AAV capsid can be a chimera either created by capsid evolution or by rational capsid engineering from a naturally isolated AAV variants to capture desirable serotype features such as enhanced or specific tissue tropism and/or a host immune response escape. Naturally isolated AAV variants include, but not limited to, AAV-DJ, AAV-HAE1, AAV-HAE2, AAVM41, AAV-1829, AAV2 Y/F, AAV2 T/V, AAV218, AAV2.5, AAV9.45, AAV9.61, AAV-B1, AAV-AS, AAV9.45A-String (e.g., AAV9.45-AS), AAV9.45Angiopep, AAV9.47-Angiopep, and AAV9.47-AS, AAV-PHP.B, AAV-PHP.eB, AAV-PHP.S, AAV-F, AAVcc.47, and AAVcc.81. In an aspect, a disclosed AAV vector can be AAV-Rh74 or a related variant (e.g., capsid variants like RHM4-1). In an aspect, a disclosed AAV vector can be rAAV9.

### 4. Formulations

Disclosed herein is a pharmaceutical formulation comprising a disclosed isolated nucleic acid molecule. Disclosed herein is a pharmaceutical formulation comprising a disclosed isolated nucleic acid molecule and a pharmaceutically acceptable carrier. Disclosed herein is a pharmaceutical formulation comprising a disclosed vector. Disclosed herein is a pharmaceutical formulation comprising a disclosed vector and a pharmaceutically acceptable carrier.

Disclosed herein is a pharmaceutical formulation comprising an isolated nucleic acid molecule for repairing the IVS1 variant of Pompe disease, comprising a nucleic acid sequence encoding a repair template, wherein the repair template comprises a first sequence having homology to a sequence in intron 1 of GAA and a second sequence having homology to a sequence in exon 2 of GAA, wherein the homologous sequences flank a promoter; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is a pharmaceutical formulation comprising an isolated nucleic acid molecule for repairing the ΔT525 variant of Pompe disease, comprising a nucleic acid sequence encoding a repair template, wherein the repair template comprises a sequence having homology to a sequence in exon 2 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is a pharmaceutical formulation comprising an isolated nucleic acid molecule for repairing the 1826DupA variant of Pompe disease, comprising a nucleic acid sequence encoding a repair template, wherein the repair template comprises a sequence having homology to a sequence in exon 13 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is a pharmaceutical formulation comprising a vector comprising an isolated nucleic acid molecule for repairing the IVS1 variant of Pompe disease, comprising a nucleic acid sequence encoding a repair template, wherein the repair template comprises a first sequence having homology to a sequence in intron 1 of GAA and a second sequence having homology to a sequence in exon 2 of GAA, wherein the homologous sequences flank a promoter; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is a pharmaceutical formulation comprising a vector comprising an isolated nucleic acid molecule for repairing the ΔT525 variant of Pompe disease, comprising a nucleic acid sequence encoding a repair template, wherein the repair template comprises a sequence having homology to a sequence in exon 2 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is a pharmaceutical formulation comprising a vector comprising an isolated nucleic acid molecule for repairing the 1826DupA variant of Pompe disease, comprising a nucleic acid sequence encoding a repair template, wherein the repair template comprises a sequence having homology to a sequence in exon 13 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9.

Disclosed herein is a pharmaceutical formulation comprising a gene editing system for stably integrating an alpha-glucosidase transgene into one or more cells, comprising a donor nucleic acid molecule; and a CRISPR nucleic acid molecule, wherein the donor nucleic acid molecule comprises a nucleic acid sequence encoding a repair template, a promoter operably linked to the repair template, and a poly A sequence; and the CRISPR nucleic acid molecule comprises a nucleic acid sequence encoding a guide RNA (gRNA) operably linked to a first promoter and a nucleic acid sequence encoding a Cas9 nuclease operably linked to second promoter.

Disclosed herein is a pharmaceutical formulation comprising a gene editing system for stably integrating an alpha-glucosidase transgene into one or more cells, comprising a first vector comprising an isolated donor nucleic acid molecule; and a second vector comprising an isolated CRISPR nucleic acid molecule, wherein the donor nucleic acid molecule comprises a nucleic acid sequence encoding a repair template, a promoter operably linked to the repair template, and a poly A sequence; and the CRISPR nucleic acid molecule comprises a nucleic acid sequence encoding a guide RNA (gRNA) operably linked to a first promoter and a nucleic acid sequence encoding a Cas9 nuclease operably linked to second promoter.

In an aspect, a disclosed pharmaceutical formulation can repair a defective GAA gene. In an aspect, a disclosed pharmaceutical formulation can restore one or more aspects of cellular homeostasis and/or cellular functionality and/or metabolic dysregulation. In an aspect, a disclosed pharmaceutical formulation can improve the efficiency of gene editing. In an aspect, a disclosed pharmaceutical formulation can be used to stably integrate a transgene (such as, for example, GAA) into one or more disclosed cells. In an aspect, a disclosed pharmaceutical formulation can be used in a method of treating a Pompe patient or in a method of validating a gene editing system.

In an aspect, a disclosed pharmaceutical formulation can restore one or more aspects of cellular homeostasis and/or cellular functionality and/or metabolic dysregulation (such as, for example, glycogen accumulation). In an aspect, a disclosed pharmaceutical formulation can restore the functionality and/or structural integrity of a missing, deficient, and/or mutant protein or enzyme (such as, for example, a mutant GAA).

In an aspect, a disclosed pharmaceutical formulation can comprise (i) one or more active agents, (ii) biologically active agents, (iii) one or more pharmaceutically active agents, (iv) one or more immune-based therapeutic agents, (v) one or more clinically approved agents, or (vi) a combination thereof. In an aspect, a disclosed composition can comprise one or more immune modulators. In an aspect, a disclosed composition can comprise one or more proteasome inhibitors. In an aspect, a disclosed composition can comprise one or more immunosuppressives or immunosuppressive agents. In an aspect, an immunosuppressive agent can be anti-thymocyte globulin (ATG), cyclosporine (CSP), mycophenolate mofetil (MMF), or a combination thereof. In an aspect, a disclosed formulation can comprise an anaplerotic agent (such as, for example, C7 compounds like triheptanoin or MCT).

In an aspect, a disclosed formulation can comprise a disclosed small molecule. In an aspect, a disclosed small molecule can assist in restoring the functionality and/or structural integrity of a missing, deficient, and/or mutant protein or enzyme.

In an aspect, any disclosed pharmaceutical formulation can comprise one or more excipients and/or pharmaceutically acceptable carriers. Excipients and/or pharmaceutically acceptable carriers are known to the art and are discussed supra.

### 5. Plasmids

Disclosed herein is a plasmid comprising one or more disclosed isolated nucleic acid molecules. Disclosed here are plasmids used in methods of making a disclosed composition such as, for example, a disclosed isolated nucleic acid molecule, a disclosed vector, or a disclosed pharmaceutical formulation. Plasmids and using plasmids are known to the art.

Disclosed herein is a plasmid comprising the sequence set forth in any one of SEQ ID NO: 01-SEQ ID NO:18. Disclosed herein is a plasmid comprising a sequence having at least 40%, 50%, 60%, 70%, 80%, 85%, 86%, 87%, 88%, 89%, 90%, 91%, 92%, 93%, 94%, 95%, 96%, 97%, 98%, 99% identity to the sequence set forth in any one of SEQ ID NO:01-SEQ ID NO:18 or a fragment thereof. Disclosed herein is a plasmid comprising a sequence having at least 40%-60%, at least 60%-80%, at least 80%-90%, or at least 90%-100% identity to the sequence set forth in any one of SEQ ID NO:01-SEQ ID NO: 18 or a fragment thereof.

In an aspect, a disclosed plasmid can restore one or more aspects of cellular homeostasis and/or cellular functionality and/or metabolic dysregulation. In an aspect, a disclosed plasmid can improve the efficiency of gene editing. In an aspect, a disclosed plasmid can be used to stably integrate a transgene (such as, for example, GAA) into one or more disclosed cells. In an aspect, a disclosed plasmid can be in a method of treating a Pompe patient or in a method of validating a gene editing system. In an aspect, a disclosed plasmid can restore one or more aspects of cellular homeostasis and/or cellular functionality and/or metabolic dysregulation (such as, for example, glycogen accumulation). In an aspect, a disclosed plasmid can restore the functionality and/or structural integrity of a missing, deficient, and/or mutant protein or enzyme (such as, for example, a mutant or defective GAA).

### 6. Cells

Disclosed herein are cells comprising a disclosed isolated nucleic acid molecule, a disclosed vector, and/or a disclosed plasmid. Disclosed herein are cells transduced by one or more disclosed viral vectors. Disclosed herein are cells transfected with one or more disclosed isolated nucleic acid molecules. Cells are known to the art and include liver, muscle, kidney, cardiac, and brain cells. In an aspect, a disclosed cell can be any cell having excessive glycogen or any cell affected by a defective GAA gene. In an aspect, a disclosed cell has been transfected with one or more nucleic acid sequences having the sequence set forth in any of SEQ ID NO:01-SEQ ID NO: 18. Techniques to achieve transfection and transduction are known to the art and using transfected or transduced cells are known to the art. In an aspect, disclosed herein are human immortalized cells lines transduced by one or more disclosed viral vectors or transfected with one or more disclosed isolated nucleic acids or disclosed plasmids. In an aspect, disclosed herein are human immortalized cells lines contacted with one or more disclosed pharmaceutical formulations. Disclosed herein are cells obtained for a subject treated with one or more disclosed isolated nucleic acid molecule, one or more disclosed vectors, one or more disclosed plasmids, or one or more disclosed pharmaceutical formulations.

### 7. Animals

Disclosed herein are animals treated with one or more disclosed isolated nucleic acid molecules, one or more disclosed vectors, one or more disclosed pharmaceutical formulations, and/or one or more disclosed plasmids (e.g., SEQ ID NO:01-SEQ ID NO:18). Transgenic animals are known to the art as are the techniques to generate transgenic animals.

### C. Methods of Repairing a Defective GAA Gene

Disclosed herein is a method of repairing a defective GAA gene, the method comprising contacting cells with a disclosed isolated nucleic acid molecule, wherein, following expression of the nucleic acid molecule, the defective GAA gene is repaired in the cells. Isolated nucleic acid molecules are disclosed supra.

Disclosed herein is a method of repairing a defective GAA gene, the method comprising contacting cells with a disclosed isolated donor nucleic acid molecule; and contacting the cells with the disclosed isolated CRISPR nucleic acid molecule; wherein, following expression of the nucleic acid molecules, the defective GAA gene is repaired in the cells. Isolated donor nucleic acid molecules are disclosed supra. Isolated CRISPR nucleic acid molecules are disclosed supra.

Disclosed herein is a method of repairing a defective GAA gene, the method comprising contacting cells with an isolated nucleic acid molecule for repairing the IVS1 variant of Pompe disease, comprising a nucleic acid sequence encoding a repair template, wherein the repair template comprises a first sequence having homology to a sequence in intron 1 of GAA and a second sequence having homology to a sequence in exon 2 of GAA, wherein the homologous sequences flank a promoter; a nucleic acid sequence for a promoter that is operably linked to a nucleic acid sequence encoding a guide RNA (gRNA); and a nucleic acid sequence encoding a Cas9 nuclease and a promoter operably linked to the Cas9, wherein, following expression of the nucleic acid molecule, the defective GAA gene is repaired in the cells.

Disclosed herein is a method of repairing a defective GAA gene, the method comprising contacting cells with an isolated nucleic acid molecule for repairing the ΔT525 variant of Pompe disease, comprising a nucleic acid sequence encoding a repair template, wherein the repair template comprises a sequence having homology to a sequence in exon 2 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9; wherein, following expression of the nucleic acid molecules, the defective GAA gene is repaired in the cells.

Disclosed herein is a method of repairing a defective GAA gene, the method comprising contacting cells with an isolated nucleic acid molecule for repairing the 1826DupA variant of Pompe disease, comprising a nucleic acid sequence encoding a repair template, wherein the repair template comprises a sequence having homology to a sequence in exon 13 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9; wherein, following expression of the nucleic acid molecules, the defective GAA gene is repaired in the cells.

Disclosed herein is a method of repairing a defective GAA gene, the method comprising administering to a subject a therapeutically effective amount of a disclosed vector comprising a disclosed isolated nucleic acid molecule, wherein, following expression of the nucleic acid molecule, the defective GAA gene is repaired in cells of the subject. Vectors and isolated nucleic acid molecules are disclosed supra.

Disclosed herein is a method of repairing a defective GAA gene, the method comprising administering to a subject a therapeutically effective amount of a disclosed vector comprising a disclosed isolated donor nucleic acid molecule; and administering to the subject a disclosed vector comprising a disclosed isolated CRISPR nucleic acid molecule; wherein, following expression of the nucleic acid molecules, the defective GAA gene is repaired in cells of the subject. Vectors are disclosed supra. Isolated donor nucleic acid molecules and isolated CRISPR nucleic acid molecules are disclosed supra.

Disclosed herein is a method of repairing a defective GAA gene, the method comprising administering to a subject a therapeutically effective amount of a disclosed vector comprising a disclosed isolated nucleic acid molecule for repairing the IVS1 variant of Pompe disease, comprising a nucleic acid sequence encoding a repair template, wherein the repair template comprises a first sequence having homology to a sequence in intron 1 of GAA and a second sequence having homology to a sequence in exon 2 of GAA, wherein the homologous sequences flank a promoter; a nucleic acid sequence for a promoter that is operably linked to a nucleic acid sequence encoding a guide RNA (gRNA); and a nucleic acid sequence encoding a Cas9 nuclease and a promoter operably linked to the Cas9, wherein, following expression of the nucleic acid molecule, the defective GAA gene is repaired in cells of the subject.

Disclosed herein is a method of repairing a defective GAA gene, the method comprising administering to a subject a therapeutically effective amount of a disclosed vector comprising a disclosed isolated nucleic acid molecule for repairing the ΔT525 variant of Pompe disease, comprising a nucleic acid sequence encoding a repair template, wherein the repair template comprises a sequence having homology to a sequence in exon 2 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9; wherein, following expression of the nucleic acid molecules, the defective GAA gene is repaired in cells of the subject.

Disclosed herein is a method of repairing a defective GAA gene, the method comprising administering to a subject a therapeutically effective amount of a disclosed vector comprising a disclosed isolated nucleic acid molecule for repairing the 1826DupA variant of Pompe disease, comprising a nucleic acid sequence encoding a repair template, wherein the repair template comprises a sequence having homology to a sequence in exon 13 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9; wherein, following expression of the nucleic acid molecules, the defective GAA gene is repaired in cells of the subject.

Disclosed herein is a method of repairing a defective GAA gene, the method comprising administering to a subject a therapeutically effective amount of a disclosed vector comprising a disclosed isolated nucleic acid molecule for any mutation or any variant leading to Pompe disease, comprising a nucleic acid sequence encoding a repair template, wherein the repair template comprises a sequence having homology to a wild-type sequence of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9; wherein, following expression of the nucleic acid molecules, the defective GAA gene is repaired in cells of the subject.

Disclosed herein is a method of repairing a defective GAA gene, the method comprising administering to a subject a disclosed gene editing system, wherein, following expression of the nucleic acid molecules, the defective GAA gene is repaired in cells of the subject. Vectors and isolated nucleic acid molecules are disclosed supra.

Disclosed herein is a method of repairing a defective GAA gene, the method comprising administering to a subject a disclosed vector comprising a disclosed gene editing system; wherein, following expression of the nucleic acid molecules, the defective GAA gene is repaired in cells of the subject. Vectors and isolated nucleic acid molecules are disclosed supra.

In an aspect, the disclosed cells can comprise brain cells, liver cells, muscle cells, or both. In an aspect, the disclosed cells can comprise any cells affected by a defective GAA gene. Defects in the GAA gene are known to the skilled person in the art. In an aspect, the disclosed cells can be any cells having a high and/or excess level of glycogen.

In an aspect, the disclosed cells can be in a subject. In an aspect, a disclosed subject can be diagnosed with or is suspected of having Pompe disease. In an aspect, a subject can have late onset Pompe disease or can have infantile onset Pompe disease.

In an aspect, a disclosed method of repairing a defective GAA gene can restore the functionality and/or structural integrity of a missing, deficient, and/or mutant protein or enzyme (such as, for example, GAA). In an aspect, a disclosed method can comprise restoring one or more aspects of cellular homeostasis and/or cellular functionality and/or metabolic dysregulation (such as, for example, glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, GAA).

In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a missing, deficient, and/or mutant protein or enzyme (such as, for example, GAA) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme such as, for example, GAA). In an aspect, restoration can be a partial or incomplete restoration. In an aspect, restoration can be complete or near complete restoration such that the level of expression, activity, and/or functionality is similar to that of a wild-type or control level.

In an aspect of a disclosed method of repairing a defective GAA gene, techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect of a disclosed method of repairing a defective GAA gene, a disclosed vector can be administered systemically or directly to the subject. In an aspect, a disclosed vector can be intravenously, subcutaneously, or intramuscularly administered to the subject.

In an aspect, a therapeutically effective amount of the vector can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method of repairing a defective GAA gene can comprise administering to the subject one or more additional therapeutic agents. In an aspect, a disclosed therapeutic agent can comprise enzyme replacement therapy, gene therapy, mRNA therapy, small molecule therapy, substrate reduction therapy, or any combination thereof.

In an aspect, a disclosed method of repairing a defective GAA gene can comprise treating the subject. In an aspect, treating the subject can comprise administering to the subject one or more agents that modulate the level of one or more differentially present cellular metabolites. In an aspect, treating the subject can comprise implementing a change in the subject's dietary intake of carbohydrates. Implementing a change in the subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject. In an aspect, treating the subject can comprise administering cornstarch to the subject, or administering glycoside to the subject, or administering one or more anaplerotic agents to the subject.

In an aspect of a disclosed method of repairing a defective GAA gene, a disclosed Cas9 nuclease can create a double-strand breaks (DSB) within or near the IVS1 variant in the GAA locus that results in a permanent integration of the repair template. In an aspect of a disclosed method of repairing a defective GAA gene, a disclosed Cas9 nuclease can create a double-strand break (DSB) on both sides of the ΔT525 variant in Exon 2 of the GAA locus that results in a permanent integration of the repair template. In an aspect of a disclosed method of repairing a defective GAA gene, a disclosed Cas9 nuclease can create a double-strand break (DSB) on both sides of the 1826DupA variant in Exon 13 of the GAA locus that results in a permanent integration of the repair template.

In an aspect, a disclosed method of repairing a defective GAA gene can improve the efficiency of gene editing. In an aspect, a disclosed method can be used to stably integrate a transgene (such as, for example, GAA) into one or more disclosed cells. In an aspect, a disclosed method can be used in a method of treating a Pompe patient or in a method of validating a gene editing system. In an aspect, a disclosed method can improve the efficiency of gene editing.

In an aspect, a disclosed method of repairing a defective GAA gene can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria. Such methods are known to the skilled person. In an aspect, a disclosed method can further comprise repeating a monitoring step.

In an aspect, a disclosed method of repairing a defective GAA gene can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of repairing a defective GAA gene can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of repairing a defective GAA gene can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of repairing a defective GAA gene can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method of repairing a defective GAA gene can further comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method of repairing a defective GAA gene can further comprise administering a β2 agonist. For example, in an aspect, a disclosed method can comprises administering a β2 agonist to increase the expression of one or more receptors for a lysosomal enzyme. In an aspect, β2 agonists include but are not limited to albuterol, clenbuterol, formoterol, indacaterol, olodaterol, salmeterol, vilanterol, and any combination thereof, growth hormones (e.g., human growth hormone), autocrine glycoprotein (e.g., Follistatin), or any combination thereof (see, e.g., U.S. Pat. No. 8,679,478 for a discussion of appropriate β2 agonists, which patent is incorporated by reference it its entirety for these teachings).

In an aspect, a disclosed method of repairing a defective GAA gene can further comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, one or more isolated nucleic acid molecules or one or more vectors can be administered concurrently or sequentially.

In an aspect, a disclosed method can further comprise diagnosing a subject with a genetic defect using one or more known methods to the skilled person, such as, for example, genotyping.

### D. Methods of Treating a Subject Having Pompe Disease

Disclosed herein is an in vivo method for treating a subject having Pompe disease, the method comprising administering to the subject a therapeutically effective amount of a disclosed vector comprising the isolated donor nucleic acid molecule; and administering to the subject therapeutically effective amount of a disclosed vector comprising the isolated CRISPR nucleic acid molecule; wherein the Cas9 creates a double-strand break (DSB) on both sides of a mutation in the GAA locus that results in a permanent integration of the repair template, thereby repairing the defect underlying Pompe disease.

Disclosed herein is an in vivo method for treating a subject having Pompe disease, the method comprising administering to the subject a therapeutically effective amount of a vector comprising an isolated donor nucleic acid molecule, comprising a nucleic acid sequence encoding a repair template, a promoter operably linked to the repair template, and a poly A sequence; and administering to the subject therapeutically effective amount of a vector comprising an isolated CRISPR nucleic acid molecule comprising a nucleic acid sequence encoding a guide RNA (gRNA) operably linked to a first promoter and a nucleic acid sequence encoding a Cas9 nuclease operably linked to second promoter; wherein the Cas9 creates a double-strand break (DSB) on both sides of a mutation in the GAA locus that results in a permanent integration of the repair template, thereby repairing the defect underlying Pompe disease.

Disclosed herein is an in vivo method for treating a subject having Pompe disease, the method comprising administering to the subject a therapeutically effective amount of a vector comprising an isolated nucleic acid molecule for repairing the IVS1 variant of Pompe disease, comprising a nucleic acid sequence encoding a repair template, wherein the repair template comprises a first sequence having homology to a sequence in intron 1 of GAA and a second sequence having homology to a sequence in exon 2 of GAA, wherein the homologous sequences flank a promoter; a nucleic acid sequence for a promoter that is operably linked to a nucleic acid sequence encoding a guide RNA (gRNA); and a nucleic acid sequence encoding a Cas9 nuclease and a promoter operably linked to the Cas9; wherein the Cas9 creates a double-strand break (DSB) on both sides of a mutation in the GAA locus that results in a permanent integration of the repair template, thereby repairing the defect underlying Pompe disease.

Disclosed herein is an in vivo method for treating a subject having Pompe disease, the method comprising administering to the subject therapeutically effective amount of a vector comprising an isolated nucleic acid molecule for repairing the ΔT525 variant of Pompe disease, comprising a nucleic acid sequence encoding a repair template, wherein the repair template comprises a sequence having homology to a sequence in exon 2 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9; wherein the Cas9 creates a double-strand break (DSB) on both sides of a mutation in the GAA locus that results in a permanent integration of the repair template, thereby repairing the defect underlying Pompe disease.

Disclosed herein is an in vivo method for treating a subject having Pompe disease, the method comprising administering to the subject therapeutically effective amount of a vector an isolated nucleic acid molecule for repairing the 1826DupA variant of Pompe disease, comprising a nucleic acid sequence encoding a repair template, wherein the repair template comprises a sequence having homology to a sequence in exon 13 of GAA; a nucleic acid sequence for a promoter that is operably linked to a nucleic acid sequence encoding a guide RNA (gRNA); and a nucleic acid sequence encoding a Cas9 nuclease and a promoter operably linked to the Cas9; wherein the Cas9 creates a double-strand break (DSB) on both sides of a mutation in the GAA locus that results in a permanent integration of the repair template, thereby repairing the defect underlying Pompe disease.

In an aspect, a disclosed in vivo method for treating a subject having Pompe disease can restore the functionality and/or structural integrity of a missing, deficient, and/or mutant protein or enzyme (such as, for example, GAA). In an aspect, a disclosed in vivo method for treating a subject having Pompe disease can comprise restoring one or more aspects of cellular homeostasis and/or cellular functionality and/or metabolic dysregulation (such as glycogen accumulation). In an aspect, restoring one or more aspect of cellular homeostasis and/or cellular functionality and/or metabolic dysregulation comprises restoring the functionality and/or structural integrity of a missing, deficient, and/or mutant protein or enzyme (such as, for example, GAA).

In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof.

In an aspect, restoring one or more aspects of cellular homeostasis can comprise improving, enhancing, restoring, and/or preserving one or more aspects of cellular structural and/or functional integrity. In an aspect, restoring the activity and/or functionality of a missing, deficient, and/or mutant protein or enzyme (such as, for example, GAA) can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme such as, for example, GAA). In an aspect, restoration can be a partial or incomplete restoration. In an aspect, restoration can be complete or near complete restoration such that the level of expression, activity, and/or functionality is similar to that of a wild-type or control level.

In an aspect of a disclosed in vivo method for treating a subject having Pompe disease, techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect, the disclosed cells can be in a subject. In an aspect, the disclosed cells can comprise brain cells, liver cells, muscle cells, or any combination thereof. In an aspect, the disclosed cells can comprise any cells affected by a defective GAA gene. Defects in the GAA gene are known to the skilled person in the art. In an aspect, the disclosed cells can be any cells having a high and/or excess level of glycogen.

In an aspect of a disclosed in vivo method for treating a subject having Pompe disease, a disclosed vector can be administered systemically or directly to the subject. In an aspect, a disclosed vector can be intravenously, subcutaneously, or intramuscularly administered to the subject.

In an aspect, a therapeutically effective amount of the vector can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed in vivo method for treating a subject having Pompe disease can comprise administering to the subject one or more additional therapeutic agents. In an aspect, a disclosed therapeutic agent can comprise enzyme replacement therapy, gene therapy, mRNA therapy, small molecule therapy, substrate reduction therapy, or any combination thereof.

In an aspect, a disclosed in vivo method for treating a subject having Pompe disease can comprise treating the subject. In an aspect, treating the subject can comprise administering to the subject one or more agents that modulate the level of one or more differentially present cellular metabolites. In an aspect, treating the subject can comprise implementing a change in the subject's dietary intake of carbohydrates. Implementing a change in the subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject. In an aspect, treating the subject can comprise administering cornstarch to the subject, or administering glycoside to the subject, or administering one or more anaplerotic agents to the subject.

In an aspect of a disclosed in vivo method for treating a subject having Pompe disease, a disclosed Cas9 nuclease can create a double-strand breaks (DSB) within or near the IVS1 variant in the GAA locus that results in a permanent integration of the repair template. In an aspect of a disclosed in vivo method for treating a subject having Pompe disease, a disclosed Cas9 nuclease can create a double-strand break (DSB) on both sides of the ΔT525 variant in Exon 2 of the GAA locus that results in a permanent integration of the repair template. In an aspect of a disclosed in vivo method for treating a subject having Pompe disease, a disclosed Cas9 nuclease can create a double-strand break (DSB) on both sides of the 1826DupA variant in Exon 13 of the GAA locus that results in a permanent integration of the repair template. In an aspect, a disclosed in vivo method for treating a subject having Pompe disease can improve the efficiency of gene editing. In an aspect, a disclosed method can be used to stably integrate a transgene (such as, for example, GAA) into one or more disclosed cells. In an aspect, a disclosed method can be used in a method of treating a Pompe patient or in a method of validating a gene editing system. In an aspect, a disclosed method can improve the efficiency of gene editing.

In an aspect, a disclosed in vivo method for treating a subject having Pompe disease can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria. Such methods are known to the skilled person. In an aspect, a disclosed method can further comprise repeating a monitoring step.

In an aspect, a disclosed in vivo method for treating a subject having Pompe disease can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed in vivo method for treating a subject having Pompe disease can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells after administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed in vivo method for treating a subject having Pompe disease can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed in vivo method for treating a subject having Pompe disease can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered to help with immune tolerance (e.g., antigen specific Treg cells to AAV).

In an aspect, a disclosed in vivo method for treating a subject having Pompe disease can further comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed in vivo method for treating a subject having Pompe disease can further comprise administering a β2 agonist. For example, in an aspect, a disclosed method can comprises administering a β2 agonist to increase the expression of one or more receptors for a lysosomal enzyme. In an aspect, β2 agonists include but are not limited to albuterol, clenbuterol, formoterol, indacaterol, olodaterol, salmeterol, vilanterol, and any combination thereof, growth hormones (e.g., human growth hormone), autocrine glycoprotein (e.g., Follistatin), or any combination thereof (see, e.g., U.S. Pat. No. 8,679,478 for a discussion of appropriate β2 agonists, which patent is incorporated by reference it its entirety for these teachings).

In an aspect, a disclosed in vivo method for treating a subject having Pompe disease can further comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, one or more isolated nucleic acid molecules or one or more vectors can be administered concurrently or sequentially.

In an aspect, a disclosed in vivo method for treating a subject having Pompe disease can further comprise diagnosing a subject with a genetic defect using one or more known methods to the skilled person, such as, for example, genotyping.

### E. Methods of Validating the Efficacy of a Gene Editing System

Disclosed herein is a method of validating the efficacy of a gene editing system, the method comprising contacting cells with a disclosed isolated nucleic acid molecule; measuring the expression of GAA in the edited cells; and comparing the resulting GAA expression level in the edited cells to the GAA expression level in control cells, wherein the gene editing system is effective when the GAA expression in the edited cells is greater than the GAA expression level in control cells.

Disclosed herein is a method of validating the efficacy of a gene editing system, the method comprising contacting cells with a disclosed isolated nucleic acid molecule; measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

Disclosed herein is a method of validating the efficacy of a gene editing system in a subject, the method comprising administering to the subject a disclosed vector comprising a disclosed isolated nucleic acid molecule; obtaining a biological sample of cells targets for editing; measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

Disclosed herein is a method of validating the efficacy of a gene editing system in a subject, the method comprising administering to the subject a disclosed vector comprising a disclosed isolated nucleic acid molecule; obtaining a biological sample of cells targets for editing; measuring the expression of a reporter gene in the edited cells; and comparing the resulting expression level of the reporter gene in the edited cells to the expression level of the reporter gene in control cells, wherein the gene editing system is effective when the expression level of the reporter gene in the edited cells is greater than the expression level of the reporter gene in control cells.

In a disclosed method, the disclosed control cells can comprise unedited cells. In an aspect, the disclosed control cells can comprise the subject's cells prior to administration of a disclosed vector, a disclosed nucleic acid molecule, or a disclosed pharmaceutical formulation. In an aspect, the disclosed control cells can comprise cells treated with a disclosed nucleic acid molecule or a disclosed vector having a scrambled gRNA.

In an aspect, measuring the expression of GAA and/or the reporter gene can comprise measuring the protein concentration of GAA and/or the reporter gene or measuring the mRNA level of GAA and/or the reporter gene. For example, in an aspect, measuring the protein concentration of GAA and/or the reporter gene comprises a protein chip analysis, an immunoassay, a ligand binding assay, a MALDI-TOF (Matrix Assisted Laser Desorption/Ionization Time of Flight Mass Spectrometry) analysis, a SELDI-TOF (Sulface Enhanced Laser Desorption/Ionization Time of Flight Mass Spectrometry) analysis, a radioimmunoassay, a radioimmunodiffusion assay, an octeroni immunodiffusion method, rocket immunoelectrophoresis, tissue immunostaining, a complement fixation assay, 2D by electrophoretic analysis, liquid chromatography-Mass Spectrometry (LC-MS), liquid chromatography-Mass Spectrometry/Mass Spectrometry (LC-MS/MS), Western blotting, ELISA (enzyme linked immunosorbent assay), or any combination thereof. Similarly, in an aspect, measuring the mRNA level of GAA and/or the reporter gene comprises a reverse transcription polymerase reaction (RT-PCR), a competitive reverse transcription polymerase reaction (Competitive RT-PCR), a real-time reverse transcription polymerization, an enzyme reaction (Real-time RT-PCR), an RNase protection assay (RPA), Northern blotting, a DNA chip, or any combination thereof.

Gene editing systems are disclosed supra. Isolated nucleic acid molecules are disclosed supra. Vectors comprising disclosed isolated nucleic acid molecules (including disclosed donor nucleic acid molecules and disclosed CRISPR nucleic acid molecules) are disclosed supra.

In an aspect, a disclosed donor nucleic acid molecule can comprise a nucleic acid sequence encoding a repair template, a promoter operably linked to the repair template, and a poly A sequence. In an aspect, a disclosed CRISPR nucleic acid molecule can comprise a nucleic acid sequence encoding a guide RNA (gRNA) operably linked to a first promoter and a nucleic acid sequence encoding a Cas9 nuclease operably linked to second promoter. In an aspect, a disclosed repair template can be for the IVS1 variant of Pompe disease, for the ΔT525 variant of Pompe disease, or for the 1826DupA variant of Pompe disease. In an aspect, a disclosed repair template can be for any mutation in the gene encoding alpha glucosidase (GAA). In an aspect, a disclosed repair template can be for any mutation in the gene encoding alpha glucosidase (GAA) or can be for one or more mutations in the gene encoding alpha glucosidase (GAA). In an aspect, a disclosed repair template can be for any mutation in the gene encoding alpha glucosidase (GAA). In an aspect, a disclosed repair template can be for any mutation in the gene encoding alpha glucosidase (GAA) or can be for one or more mutations in the gene encoding alpha glucosidase (GAA).

In an aspect, a disclosed repair template comprises about 300 bp to about 700 bp. In an aspect, a disclosed repair template can comprise about 400 bp, or about 600 bp, or about 650 bp.

In an aspect, a disclosed repair template can comprise the sequence set forth in SEQ ID NO: 26, SEQ ID NO:27, and SEQ ID NO:28, or a sequence having about 50%, about 60%, about 70% about 80%, about 90%, about 95%, or more identity to the sequence set forth in SEQ ID NO: 26, SEQ ID NO:27, and SEQ ID NO:28. In an aspect, a disclosed repair template can comprise a nucleic acid sequence encoding wild-type GAA or a portion thereof. In an aspect, a disclosed GAA can comprise the sequence set forth in SEQ ID NO:62, or a sequence having about 50%, about 60%, about 70% about 80%, about 90%, about 95%, or more identity to the sequence set forth in SEQ ID NO:62.

In an aspect, a disclosed repair template can comprise a restriction site. Restriction enzymes are known to the art.

For example, a disclosed repair template can comprise a first sequence having homology to a sequence in intron 1 of GAA and a second sequence having homology to a sequence in exon 2 of GAA. In an aspect, a disclosed first homologous sequence can comprise the sequence of a wild-type GAA or a portion thereof, and a disclosed second homologous sequence can comprise the sequence of a wild-type GAA or a portion thereof, or any combination thereof. In an aspect, a disclosed GAA can comprise the sequence set forth in SEQ ID NO:62.

In an aspect, a disclosed repair template can comprise a promoter sequence between the first homologous sequence and the second homology sequence. In an aspect, a disclosed promoter can comprise a CMV promoter, a EF1a promoter, a chicken beta-actin promoter, a liver-specific promoter, or a G6PC promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect, a disclosed first homologous sequence can comprise about 175 bp to about 225 bp or about 200 bp to about 220 bp. For example, in an aspect, a disclosed first homologous sequence can comprise the sequence set forth in SEQ ID NO:54, SEQ ID NO:55, SEQ ID NO:56, or SEQ ID NO:57. In an aspect, a disclosed second homologous sequence can comprise about 175 bp to about 225 bp or about 200 bp to about 220 bp. In an aspect, a disclosed second homologous can comprise the sequence set forth in SEQ ID NO:58, SEQ ID NO:59, SEQ ID NO: 60, or SEQ ID NO:61. In an aspect, a disclosed first homologous sequence can comprise the sequence of a wild-type GAA or a portion thereof, a disclosed second homologous sequence can comprise the sequence of a wild-type GAA or a portion thereof. In an aspect, a disclosed GAA can comprise the sequence set forth in SEQ ID NO:62.

In an aspect, a disclosed repair template can comprise a sequence having homology to a sequence in exon 2 of GAA. In an aspect, a disclosed repair template can comprise a sequence having homology to a sequence in exon 13 of GAA.

In an aspect of a disclosed gene editing system, a disclosed CRISPR isolated nucleic acid molecule can comprise a nucleic acid sequence encoding a promoter that is operably linked to a nucleic acid sequence encoding a guide RNA (gRNA) and drives the expression of the gRNA. In an aspect, a disclosed gRNA can target a PAM sequence at or near the IVS1 variant near the boundary between intron 1 and exon 2 in the GAA gene, a PAM sequence at or near the ΔT525 variant in Exon 2 in the GAA gene, or a PAM sequence at or near the m1826DupA variant in Exon 13 in the GAA gene. In an aspect, a disclosed gRNA can target a PAM sequence at or near any mutation in the GAA gene.

In an aspect, a disclosed gRNA can comprise the sequence set forth in any of SEQ ID NO: 29-SEQ ID NO:36.

In an aspect, a disclosed promoter operably linked to a disclosed gRNA can comprise a RNA polymerase III (Poly III) promoter. In an aspect, a disclosed Poly III promoter can comprise a U3 promoter, a U6 promoter, or a H1 promoter. In an aspect, a disclosed promoter operably linked to a disclosed gRNA can comprise a glutamine tRNA promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO:40-SEQ ID NO:48.

In an aspect of a disclosed gene editing system, a disclosed CRISPR isolated nucleic acid molecule can comprise nucleic acid sequence encoding a Cas9 nuclease and a promoter operably linked to the Cas9. In an aspect, a disclosed Cas9 nuclease can comprise a SaCas9 nuclease. For example, in an aspect, a disclosed SaCas9 nuclease can comprise the sequence set forth in SEQ ID NO: 19 or a sequence having about 60%, about 65%, about 70%, about 75%, about 80%, about 85%, about 90%, about 95%, or more than 95% identity to the sequence set forth in SEQ ID NO: 19.

In an aspect, a disclosed Cas9 nuclease can create a double-strand break (DSB) at or near the IVS1 variant in the GAA locus that results in a permanent integration of the repair template, or a double-strand break (DSB) at or near the ΔT525 variant in Exon 2 of the GAA locus that results in a permanent integration of the repair template, a double-strand break (DSB) at or near the 1826DupA variant in Exon 13 of the GAA locus that results in a permanent integration of the repair template. In an aspect, a disclosed Cas9 nuclease can create a double-strand break (DSB) at or near any mutation in the GAA locus that results in a permanent integration of the repair template.

In an aspect, a disclosed promoter operably linked the Cas9 nuclease can drive expression of the Cas9 nuclease. In an aspect, a disclosed promoter that drives expression of the Cas9 nuclease can comprise a CMV promoter, a EF1a promoter, a chicken beta-actin promoter, a liver-specific promoter, or a G6PC promoter, or a minimal CMV promoter, a minimal EF1a promoter, a minimal chicken beta-actin promoter, a minimal liver-specific promoter, or a minimal G6PC promoter. In an aspect, a disclosed promoter can comprise the sequence set forth in any one of SEQ ID NO:40-SEQ ID NO:48, or a sequence having about 50%, about 60%, about 70% about 80%, about 90%, about 95%, or more identity to the sequence set forth in any one of SEQ ID NO: 40-SEQ ID NO:48.

In an aspect of a disclosed gene editing system, a disclosed CRISPR nucleic acid molecule can comprise a nuclear localization signal (NLS). In an aspect, a disclosed NLS can comprise the sequence set forth in SEQ ID NO:50 or SEQ ID NO:51. In an aspect of a disclosed gene editing system, a disclosed CRISPR nucleic acid molecule can comprise one or more inverted terminal repeats (ITRs). In an aspect, a disclosed ITR can be derived from AAV2 or AAV9. In an aspect, a disclosed ITR can comprise the sequence set forth in any one of SEQ ID NO:20, SEQ ID NO:21, or SEQ ID NO:22. In an aspect of a disclosed gene editing system, a disclosed CRISPR nucleic acid molecule can comprise a poly A sequence. In an aspect, a disclosed polyA sequence can comprise the sequence set forth in SEQ ID NO:52 or SEQ ID NO:53. In an aspect of a disclosed gene editing system, a disclosed CRISPR nucleic acid molecule can comprise one or more hemagglutinin (HA) tags. In an aspect, a disclosed HA tag can comprise the sequence set forth in SEQ ID NO:23 or SEQ ID NO:24. In an aspect, a disclosed isolated nucleic acid molecule can comprise a CRISPR/transactivating RNA (chRNA) sequence. In an aspect, a chRNA sequence can comprise the sequence set forth in SEQ ID NO:25.

In an aspect, a disclosed gene editing system can restore one or more aspects of cellular homeostasis and/or cellular functionality and/or metabolic dysregulation. In an aspect, a disclosed gene editing system can improve the efficiency of gene editing. In an aspect, a disclosed gene editing system can be used to stably integrate a transgene (such as, for example, GAA) into one or more disclosed cells. In an aspect, a disclosed gene editing system can be used in a method of treating a Pompe patient or in a method of validating a gene editing system.

In an aspect, a disclosed gene editing system can restore one or more aspects of cellular homeostasis and/or cellular functionality and/or metabolic dysregulation (such as, for example, glycogen accumulation). In an aspect, a disclosed gene editing system can restore the functionality and/or structural integrity of a missing, deficient, and/or mutant protein or enzyme (such as, for example, a mutant GAA).

In an aspect, a disclosed method of validating the efficacy of a gene editing system can further comprise administering a β2 agonist. For example, in an aspect, a disclosed method can comprises administering a β2 agonist to increase the expression of one or more receptors for a lysosomal enzyme. In an aspect, β2 agonists include but are not limited to albuterol, clenbuterol, formoterol, indacaterol, olodaterol, salmeterol, vilanterol, and any combination thereof, growth hormones (e.g., human growth hormone), autocrine glycoprotein (e.g., Follistatin), or any combination thereof (see, e.g., U.S. Pat. No. 8,679,478 for a discussion of appropriate β2 agonists, which patent is incorporated by reference it its entirety for these teachings).

In an aspect, a disclosed method of validating the efficacy of a gene editing system can comprise modifying one or more aspects of the method. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, one or more isolated nucleic acid molecules or one or more vectors can be administered concurrently or sequentially.

### F. Methods of Treating and/or Preventing a Genetic Disease or Disorder

Disclosed herein is a method of repairing a genetic defect, the method comprising contacting cells with an isolated nucleic acid molecule, wherein, following expression of the nucleic acid molecule, the genetic defect is repaired in the cells.

Disclosed herein is a method of repairing a genetic defect, the method comprising contacting cells with an isolated donor nucleic acid molecule; and contacting the cells with an isolated CRISPR nucleic acid molecule; wherein, following expression of the nucleic acid molecules, the genetic defect is repaired in the cells.

Disclosed herein is a method of repairing a genetic defect, the method comprising administering to a subject a therapeutically effective amount of a disclosed vector comprising a disclosed isolated nucleic acid molecule, wherein, following expression of the nucleic acid molecule, the genetic defect gene is repaired in cells of the subject.

Disclosed herein is a method of repairing a genetic defect, the method comprising administering to a subject a therapeutically effective amount of a disclosed vector comprising a disclosed isolated donor nucleic acid molecule; and administering to the subject a disclosed vector comprising a disclosed isolated CRISPR nucleic acid molecule; wherein, following expression of the nucleic acid molecules, the genetic defect gene is repaired in cells of the subject.

Genetic diseases and disorders include, but are not limited to, diseases and disorders due to a defect in the following genes: ABCA1, ABCA12, ABCA13, ABCA2, ABCA3, ABCA4, ABCA5, ABCC1, ABCC2, ABCC6, ABCC8, ABCC9, ACAN, ADAMTS13, ADCY10, ADGRV1, AGL, AGRN, AHDC1, ALK, ALMS1, ALPK3, ALS2, ANAPC1, ANK1, ANK2, ANK3, ANKRD11, ANKRD26, APC, APC2, APOB, ARFGEF2, ARHGAP31, ARHGEF10, ARHGEF18, ARID1A, ARID1B, ARID2, ASH1L, ASPM, ASXL1, ASXL2, ASXL3, ATM, ATP7A, ATP7B, ATR, ATRX, BAZ1A, BAZ2B, BCOR, BCORL1, BDP1, BLM, BPTF, BRCA1, BRCA2, BRD4, BRWD3, C2CD3, C3, C5, CACNA1A, CACNA1B, CACNA1C, CACNA1D, CACNA1E, CACNA1F, CACNA1G, CACNA1H, CACNA1S, CAD, CAMTA1, CARMIL2, CC2D2A, CCDC88A, CCDC88C, CCNB3, CDH23, CDK13, CDK5RAP2, CELSR1, CEMIP2, CENPE, CENPF, CENPJ, CEP152, CEP164, CEP250, CEP290, CFAP43, CFAP44, CFAP65, CFTR/ABCC7, CHD1, CHD2, CHD3, CHD4, CHD7, CHD8, CIC, CIT, CLIP1, CLTC, CNOT1, CNTNAP1, COL11A1, COL11A2, COL12A1, COL17A1, COL18A1, COL1A1, COL1A2, COL27A1, COL2A1, COL3A1, COL4A1, COL4A2, COL4A3, COL4A4, COL4A5, COL4A6, COL5A1, COL5A2, COL6A3, COL7A1, CPAMD8, CPLANE1, CPS1, CPSF1, CRB1, CREBBP, CUBN, CUL7, CUX1, DCC, DCHS1, DEPDC5, DICER1, DIP2B, DLC1, DMD, DMXL2, DNAH1, DNAH11, DNAH17, DNAH2, DNAH5, DNAH7, DNAH8, DNAH9, DNMBP, DNMT1, DOCK2, DOCK3, DOCK6, DOCK7, DOCK8, DSCAM, DSP, DST, DUOX2, DYNC1H1, DYNC2H1, DYSF, EIF2AK4, EP300, EPG5, ERCC6, ERCC6L2, EXPH5, EYS, F5, F8, FANCA, FANCD2, FANCM, FAT1, FAT4, FBN1, FBN2, FLG, FLG2, FLNA, FLNB, FLNC, FLT4, FMN2, FN1, FRAS1, FREM1, FREM2, FSIP2, FYCO1, GLI2, GLI3, GPR179, GREB1L, GRIN2A, GRIN2B, GRIN2D, HCFC1, HECW2, HERC1, HERC2, HFM1, HIVEP1, HIVEP2, HMCN1, HSPG2, HTT, HUWE1, HYDIN, IFT140, IFT172, IGF1R, IGF2R, IGSF1, INSR, INTS1, IQSEC2, ITGB4, ITPR1, ITPR2, JMJD1C, KALRN, KANK1, KAT6A, KAT6B, KDM3B, KDM5B, KDM5C, KDM6A, KDM6B, KDR, KIAA0586, KIAA1109, KIAA1549, KIDINS220, KIF14, KIF1A, KIF1B, KIF21A, KIF26B, KIF7, KMT2A, KMT2B, KMT2C, KMT2D, KMT2E, KNL1, LAMA1, LAMA2, LAMA3, LAMA4, LAMA5, LAMB1, LAMB2, LAMC3, LCT, LOXHD1, LPA, LRBA, LRP1, LRP2, LRP4, LRP5, LRP6, LRPPRC, LRRK1, LRRK2, LTBP2, LTBP4, LYST, MACF1, MADD, MAGI2, MAP1B, MAP3K1, MAPK8IP3, MAPKBP1, MAST1, MBD5, MCM3AP, MED12, MED12L, MED13, MED13L, MED23, MEGF8, MET, MLH3, MPDZ, MSH6, MTOR, MYH10, MYH11, MYH14, MYH2, MYH3, MYH6, MYH7, MYH7B, MYH8, MYH9, MYLK, MY015A, MYO18B, MYO3A, MYOSA, MYOSB, MYO7A, MYO9A, NALCN, NBAS, NBEA, NBEAL2, NCAPD2, NCAPD3, NEB, NEXMIF, NEXMIF, NF1, NFASC, NHS, NIN, NIPBL, NLRP1, NOTCH1, NOTCH2, NOTCH3, NPHP4, NRXN1, NRXN3, NSD1, NSD2, NUP155, NUP188, NUP205, OBSCN, OBSL1, OTOF, OTOG, OTOGL, PARD3, PBRM1, PCDH15, PCLO, PCNT, PHIP, PI4KA, PIEZO1, PIEZO2, PIK3C2A, PIKFYVE, PKD1, PKD1L1, PKHD1, PLCE1, PLEC, PLEKHG2, PNPLA6, POGZ, POLA1, POLE, POLR1A, POLR2A, POLR3A, PRG4, PRKDC, PRPF8, PRR12, PRX, PTCH1, PTPN23, PTPRF, PTPRJ, PTPRQ, PXDN, QRICH2, RAB3GAP2, RAI1, RALGAPA1, RANBP2, RB1CC1, RELN, RERE, REV3L, RIC1, RIMS1, RIMS2, RNF213, ROBO1, ROBO2, ROBO3, ROS1, RP1, RP1L1, RTTN, RUSC2, RYR1, RYR2, SACS, SAMD9, SAMD9L, SBF2, SCAPER, SCN10A, SCN11A, SCN1A, SCN2A, SCN3A, SCN4A, SCN5A, SCN8A, SCN9A, SETBP1, SETD1A, SETD1B, SETD2, SETD5, SETX, SHANK2, SHANK3, SHROOM4, SI, SIPA1L3, SLIT2, SLX4, SMARCA2, SMARCA4, SMCHD1, SNRNP200, SON, SPEF2, SPEG, SPG11, SPTA1, SPTAN1, SPTB, SPTBN2, SPTBN4, SRCAP, STRC, SVIL, SYNE1, SYNGAP1, SYNJ1, SZT2, TAF1, TANC2, TCF20, TCOF1, TDRD9, TECPR2, TECTA, TENM3, TENM4, TET3, TEX14, TEX15, TG, THOC2, TMEM94, TNC, TNIK, TNR, TNRC6B, TNXB, TOGARAM1, TONSL, TRIO, TRIOBP, TRIP11, TRIP12, TRPM1, TRPM6, TRPM7, TRRAP, TSC2, TTC37, TTN, TUBGCP6, UBR1, UNC80, USH2A, USP9X, VCAN, VPS13A, VPS13B, VPS13C, VPS13D, VWF, WDFY3, WDR19, WDR62, WDR81, WNK1, WRN, ZFHX2, ZFYVE26, ZNF142, ZNF292, ZNF335, ZNF407, ZNF462, ZNF469, or a portion thereof.

Genetic diseases and disorders can also include, but are not limited to, diseases and disorders due to a defect in the following gene: dystrophin including mini- and micro-dystrophins (DMD); titin (TTN); titin cap (TCAP) α-sarcoglycan (SGCA), β-sarcoglycan (SGCB), γ-sarcoglycan (SGCG) or δ-sarcoglycan (SGCD); alpha-1-antitrypsin (A1-AT); myosin heavy chain 6 (MYH6); myosin heavy chain 7 (MYH7); myosin heavy chain 11 (MYH11); myosin light chain 2 (ML2); myosin light chain 3 (ML3); myosin light chain kinase 2 (MYLK2); myosin binding protein C (MYBPC3); desmin (DES); dynamin 2 (DNM2); laminin α2 (LAMA2); lamin A/C (LMNA); lamin B (LMNB); lamin B receptor (LBR); dysferlin (DYSF); emerin (EMD); insulin; blood clotting factors, including but not limited to, factor VIII and factor IX; erythropoietin (EPO); lipoprotein lipase (LPL); sarcoplasmic reticulum Ca2++-ATPase (SERCA2A), S100 calcium binding protein A1 (S100A1); myotubularin (MTM); DM1 protein kinase (DMPK); glycogen phosphorylase L (PYGL); glycogen phosphorylase, muscle associated (PYGM); glycogen synthase 1 (GYS1); glycogen synthase 2 (GYS2); α-galactosidase A (GLA); α-N-acetylgalactosaminidase (NAGA); acid α-glucosidase (GAA), sphingomyelinase phosphodiesterase 1 (SMPD1); lysosomal acid lipase (LIPA); collagen type I α1 chain (COL1A1); collagen type I α2 chain (COL1A2); collagen type III α1 chain (COL3A1); collagen type V α1 chain (COL5A1); collagen type V α2 chain (COL5A2); collagen type VI α1 chain (COL6A1); collagen type VI α2 chain (COL6A2); collagen type VI α3 chain (COL6A3); procollagen-lysine 2-oxoglutarate 5-dioxygenase (PLOD1); lysosomal acid lipase (LIPA); frataxin (FXN); myostatin (MSTN); β-N-acetyl hexosaminidase A (HEXA); β-N-acetylhexosaminidase B (HEXB); β-glucocerebrosidase (GBA); adenosine monophosphate deaminase 1 (AMPDI); β-globin (HBB); iduronidase (IDUA); iduronate 2-sulfate (IDS); troponin 1 (TNNI3); troponin T2 (TNNT2); troponin C (TNNC1); tropomyosin 1 (TPM1); tropomyosin 3 (TPM3); N-acetyl-α-glucosaminidase (NAGLU); N-sulfoglucosamine sulfohydrolase (SGSH); heparan-α-glucosaminide N-acetyltransferase (HGSNAT); integrin a 7 (IGTA7); integrin a 9 (IGTA9); glucosamine (N-acetyl)-6-sulfatase (GNS); galactosamine (N-acetyl)-6-sulfatase (GALNS); β-galactosidase (GLB1); β-glucuronidase (GUSB); hyaluronoglucosaminidase 1 (HYAL1); acid ceramidase (ASAHI); galactosylcermidase (GALC); cathepsin A (CTSA); cathepsin D (CTSA); cathepsin K (CTSK); GM2 ganglioside activator (GM2A); arylsulfatase A (ARSA); arylsulfatase B (ARSB); formylglycine-generating enzyme (SUMFI); neuraminidase 1 (NEU1); N-acetylglucosamine-1-phosphate transferase a (GNPTA); N-acetylglucosamine-1-phosphate transferase β (GNPTB); N-acetylglucosamine-1-phosphate transferase γ (GNPTG); mucolipin-1 (MCOLN1); NPC intracellular transporter 1 (NPC1); NPC intracellular transporter 2 (NPC2); ceroid lipofuscinosis 5 (CLN5); ceroid lipofuscinosis 6 (CLN6); ceroid lipofuscinosis 8 (CLN8); palmitoyl protein thioesterase 1 (PPT1); tripeptidyl peptidase 1 (TPP1); battenin (CLN3); DNAJ heat shock protein family 40 member C5 (DNAJC5); major facilitator superfamily domain containing 8 (MFSD8); mannosidase a class 2B member 1 (MAN2B1); mannosidase R (MANBA); aspartylglucosaminidase (AGA); α-L-fucosidase (FUCA1); cystinosin, lysosomal cysteine transporter (CTNS); sialin; solute carrier family 2 member 10 (SLC2A10); solute carrier family 17 member 5 (SLC17A5); solute carrier family 6 member 19 (SLC6A19); solute carrier family 22 member 5 (SLC22A5); solute carrier family 37 member 4 (SLC37A4); lysosomal associated membrane protein 2 (LAMP2); sodium voltage-gated channel α subunit 4 (SCN4A); sodium voltage-gated channel β subunit 4 (SCN4B); sodium voltage-gated channel α subunit 5 (SCN5A); sodium voltage-gated channel α subunit 4 (SCN4A); calcium voltage-gated channel subunit α1c (CACNA1C); calcium voltage-gated channel subunit α1s (CACNA1S); phosphoglycerate kinase 1 (PGK1); phosphoglycerate mutase 2 (PGAM2); amylo-α-1,6-glucosidase,4-α-glucanotransferase (AGL); potassium voltage-gated channel ISK-related subfamily member 1 (KCNE1); potassium voltage-gated channel ISK-related subfamily member 2 (KCNE2); potassium voltage-gated channel subfamily J member 2 (KCNJ2); potassium voltage-gated channel subfamily J member 5 (KCNJ5); potassium voltage-gated channel subfamily H member 2 (KCNH2); potassium voltage-gated channel KQT-like subfamily member 1 (KCNQ1); hyperpolarization-activated cyclic nucleotide-gated potassium channel 4 (HCN4); chloride voltage-gated channel 1 (CLCN1); carnitine palmitoyltransferase 1A (CPT1A); ryanodine receptor 1 (RYR1); ryanodine receptor 2 (RYR2); bridging integrator 1 (BIN1); LARGE xylosyl- and glucuronyltransferase 1 (LARGE1); docking protein 7 (DOK7); fukutin (FKTN); fukutin related protein (FKRP); selenoprotein N (SELENON); protein O-mannosyltransferase 1 (POMT1); protein O-mannosyltransferase 2 (POMT2); protein O-linked mannose N-acetylglucosaminyltransferase 1 (POMGNT1); protein O-linked mannose N-acetylglucosaminyltransferase 2 (POMGNT2); protein-O-mannose kinase (POMK); isoprenoid synthase domain containing (ISPD); plectin (PLEC); cholinergic receptor nicotinic epsilon subunit (CHRNE); choline O-acetyltransferase (CHAT); choline kinase β (CHKB); collagen like tail subunit of asymmetric acetylcholinesterase (COLQ); receptor associated protein of the synapse (RAPSN); four and a half LIM domains 1 (FHL1); β-1,4-glucuronyltransferase 1 (B4GAT1); β-1,3-N-acetylgalactosaminyltransferase 2 (B3GALNT2); dystroglycan 1 (DAGI); transmembrane protein 5 (TMEM5); transmembrane protein 43 (TMEM43); SECIS binding protein 2 (SECISBP2); glucosamine (UDP-N-acetyl)-2-epimerase/N-acetylmannosamine kinase (GNE); anoctamin 5 (ANO5); structural maintenance of chromosomes flexible hinge domain containing 1 (SMCHD1); lactate dehydrogenase A (LDHA); lactate dehydrogenase B (LHDB); calpain 3 (CAPN3); caveolin 3 (CAV3); tripartite motif containing 32 (TRIM32); CCHC-type zinc finger nucleic acid binding protein (CNBP); nebulin (NEB); actin, α1, skeletal muscle (ACTA1); actin, α1, cardiac muscle (ACTC1); actinin α2 (ACTN2); poly(A)-binding protein nuclear 1 (PABPN1); LEM domain-containing protein 3 (LEMD3); zinc metalloproteinase STE24 (ZMPSTE24); microsomal triglyceride transfer protein (MTTP); cholinergic receptor nicotinic α1 subunit (CHRNA1); cholinergic receptor nicotinic α2 subunit (CHRNA2); cholinergic receptor nicotinic α3 subunit (CHRNA3); cholinergic receptor nicotinic α4 subunit (CHRNA4); cholinergic receptor nicotinic α5 subunit (CHRNA5); cholinergic receptor nicotinic α6 subunit (CHRNA6); cholinergic receptor nicotinic α7 subunit (CHRNA7); cholinergic receptor nicotinic α8 subunit (CHRNA8); cholinergic receptor nicotinic α9 subunit (CHRNA9); cholinergic receptor nicotinic α10 subunit (CHRNA10); cholinergic receptor nicotinic β1 subunit (CHRNB1); cholinergic receptor nicotinic β2 subunit (CHRNB2); cholinergic receptor nicotinic β3 subunit (CHRNB3); cholinergic receptor nicotinic β4 subunit (CHRNB4); cholinergic receptor nicotinic γ subunit (CHRNG1); cholinergic receptor nicotinic a subunit (CHRND); cholinergic receptor nicotinic E subunit (CHRNE1); ATP binding cassette subfamily A member 1 (ABCA1); ATP binding cassette subfamily C member 6 (ABCC6); ATP binding cassette subfamily C member 9 (ABCC9); ATP binding cassette subfamily D member 1 (ABCD1); ATPase sarcoplasmic/endoplasmic reticulum Ca2+ transporting 1 (ATP2A1); ATM serine/threonine kinase (ATM); a tocopherol transferase protein (TTPA); kinesin family member 21A (KIF21A); paired-like homeobox 2a (PHOX2A); heparan sulfate proteoglycan 2 (HSPG2); stromal interaction molecule 1 (STIM1); notch 1 (NOTCHI); notch 3 (NOTCH3); dystrobrevin a (DTNA); protein kinase AMP-activated, noncatalytic γ2 (PRKAG2); cysteine- and glycine-rich protein 3 (CSRP3); viniculin (VCL); myozenin 2 (MyoZ2); myopalladin (MYPN); junctophilin 2 (JPH2); phospholamban (PLN); calreticulin 3 (CALR3); nexilin F-actin-binding protein (NEXN); LIM domain binding 3 (LDB3); eyes absent 4 (EYA4); huntingtin (HTT); androgen receptor (AR); protein tyrosine phosphate non-receptor type 11 (PTPN11); junction plakoglobin (JUP); desmoplakin (DSP); plakophilin 2 (PKP2); desmoglein 2 (DSG2); desmocollin 2 (DSC2); catenin α3 (CTNNA3); NK2 homeobox 5 (NKX2-5); A-kinase anchor protein 9 (AKAP9); A-kinase anchor protein 10 (AKAP10); guanine nucleotide-binding protein α-inhibiting activity polypeptide 2 (GNA12); ankyrin 2 (ANK2); syntrophin α-1 (SNTAT); calmodulin 1 (CALM1); calmodulin 2 (CALM2); HTRA serine peptidase 1 (HTRA1); fibrillin 1 (FBN1); fibrillin 2 (FBN2); xylosyltransferase 1 (XYLT1); xylosyltransferase 2 (XYLT2); tafazzin (TAZ); homogentisate 1,2-dioxygenase (HGD); glucose-6-phosphatase catalytic subunit (G6PC); 1,4-alpha-glucan enzyme 1 (GBE1); phosphofructokinase, muscle (PFKM); phosphorylase kinase regulatory subunit alpha 1 (PHKA1); phosphorylase kinase regulatory subunit alpha 2 (PHKA2); phosphorylase kinase regulatory subunit beta (PHKB); phosphorylase kinase catalytic subunit gamma 2 (PHKG2); phosphoglycerate mutase 2 (PGAM2); cystathionine-beta-synthase (CBS); methylenetetrahydrofolate reductase (MTHFR); 5-methyltetrahydrofolate-homocysteine methyltransferase (MTR); 5-methyltetrahydrofolate-homocysteine methyltransferase reductase (MTRR); methylmalonic aciduria and homocystinuria, cblD type (MMADHC); mitochondrial DNA, including, but not limited to mitochondrially encoded NADH: ubiquinone oxidoreductase core subunit 1 (MT-ND1); mitochondrially encoded NADH: ubiquinone oxidoreductase core subunit 5 (MT-ND5); mitochondrially encoded tRNA glutamic acid (MT-TE); mitochondrially encoded tRNA histadine (MT-TH); mitochondrially encoded tRNA leucine 1 (MT-TL1); mitochondrially encoded tRNA lysine (MT-TK); mitochondrially encoded tRNA serine 1 (MT-TS1); mitochondrially encoded tRNA valine (MT-TV); mitogen-activated protein kinase 1 (MAP2K1); B-Raf proto-oncogene, serine/threonine kinase (BRAF); raf-1 proto-oncogene, serine/threonine kinase (RAF1); growth factors, including, but not limited to insulin growth factor 1 (IGF-1); transforming growth factor β3 (TGFβ3); transforming growth factor β receptor, type I (TGFβR1); transforming growth factor β receptor, type II (TGFβR2), fibroblast growth factor 2 (FGF2), fibroblast growth factor 4 (FGF4), vascular endothelial growth factor A (VEGF-A), vascular endothelial growth factor B (VEGF-B); vascular endothelial growth factor C (VEGF-C), vascular endothelial growth factor D (VEGF-D), vascular endothelial growth factor receptor 1 (VEGFR1), and vascular endothelial growth factor receptor 2 (VEGFR2); interleukins; immunoadhesins; cytokines; and antibodies.

In an aspect, the disclosed cells can comprise brain cells, liver cells, muscle cells, or both. In an aspect, the disclosed cells can comprise any cells affected by a defective gene. Defective gene are known to the skilled person in the art.

In an aspect, the disclosed cells can be in a subject. In an aspect, a disclosed subject can be diagnosed with or is suspected of having a genetic disease or disorder. In an aspect, a subject can have or be suspected of having a disease or disorder that can be treated with gene therapy. Examples of such diseases or disorder can include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders, Alzheimer's disease (GDF; neprilysin), multiple sclerosis (β-interferon), Parkinson's disease (glial-cell line derived neurotrophic factor [GDNF]), Huntington's disease (RNAi to remove repeats), amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatin, myostatin propeptide, follistatin, activin type II soluble receptor, anti-inflammatory polypeptides such as the I-kappa B dominant mutant, sarcospan, utrophin, mini-utrophin, antisense or RNAi against splice junctions in the dystrophin gene to induce exon skipping (see, e.g., WO 2003/095647), antisense against U7 snRNAs to induce exon skipping (see, e.g., WO 2006/021724), and antibodies or antibody fragments against myostatin or myostatin propeptide) and Becker, Gaucher disease (glucocerebrosidase), Hurler's disease (α-L-iduronidase), adenosine deaminase deficiency (adenosine deaminase), glycogen storage diseases (e.g., Fabry disease [a-galactosidase] and Pompe disease [lysosomal acid α-glucosidase]) and other metabolic disorders, congenital emphysema (α1-antitrypsin), Lesch-Nyhan Syndrome (hypoxanthine guanine phosphoribosyl transferase), Niemann-Pick disease (sphingomyelinase), Tay Sachs disease (lysosomal hexosaminidase A), Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor I (I-1) and fragments thereof (e.g., IIC), serca2a, zinc finger proteins that regulate the phospholamban gene, Barkct, P2-adrenergic receptor, p2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as IRAP and TNFa soluble receptor), hepatitis (a-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like

In an aspect, a disclosed method of repairing a genetic defective can restore the functionality and/or structural integrity of a missing, deficient, and/or mutant protein or enzyme. In an aspect, a disclosed method can comprise restoring one or more aspects of cellular homeostasis and/or cellular functionality and/or metabolic dysregulation. In an aspect, restoring one or more aspect of cellular homeostasis and/or cellular functionality and/or metabolic dysregulation comprises restoring the functionality and/or structural integrity of a missing, deficient, and/or mutant protein or enzyme.

In an aspect, restoring one or more aspects of cellular homeostasis and/or cellular functionality can comprise one or more of the following: (i) correcting cell starvation in one or more cell types; (ii) normalizing aspects of the autophagy pathway (such as, for example, correcting, preventing, reducing, and/or ameliorating autophagy); (iii) improving, enhancing, restoring, and/or preserving mitochondrial functionality and/or structural integrity; (iv) improving, enhancing, restoring, and/or preserving organelle functionality and/or structural integrity; (v) correcting enzyme dysregulation; (vi) reversing, inhibiting, preventing, stabilizing, and/or slowing the rate of progression of the multi-systemic manifestations of a genetic disease or disorder; (vii) reversing, inhibiting, preventing, stabilizing, and/or slowing the rate of progression of a genetic disease or disorder, or (viii) any combination thereof. In an aspect, restoring one or more aspects of cellular homeostasis can comprise improving, enhancing, restoring, and/or preserving one or more aspects of cellular structural and/or functional integrity.

In an aspect, restoring the activity and/or functionality of a disclosed missing, deficient, and/or mutant protein or enzyme can comprise a 10%, 20%, 30%, 40%, 50%, 60%, 70%, 80%, 90%, 100%, or any amount of restoration when compared to a pre-existing level such as, for example, a pre-treatment level. In an aspect, the amount of restoration can be 10-20%, 20-30%, 30-40%, 40-50%, 50-60%, 60-70%, 70-80%, 80-90%, or 90-100% more than a pre-existing level such as, for example, a pre-treatment level. In an aspect, restoration can be measured against a control level or a reference level (e.g., determined, for example, using one or more subjects not having a missing, deficient, and/or mutant protein or enzyme). In an aspect, restoration can be a partial or incomplete restoration. In an aspect, restoration can be complete or near complete restoration such that the level of expression, activity, and/or functionality is similar to that of a wild-type or control level.

In an aspect of a disclosed method of repairing a disclosed defective gene, techniques to monitor, measure, and/or assess the restoring one or more aspects of cellular homeostasis and/or cellular functionality can comprise qualitative (or subjective) means as well as quantitative (or objective) means. These means are known to the skilled person.

In an aspect of a disclosed method of repairing a disclosed defective gene, a disclosed vector can be administered systemically or directly to the subject. In an aspect, a disclosed vector can be intravenously, subcutaneously, or intramuscularly administered to the subject.

In an aspect, a therapeutically effective amount of the vector can comprise a range of about 1×10¹⁰ vg/kg to about 2×10¹⁴ vg/kg. In an aspect, for example, a disclosed vector can be administered at a dose of about 1×10¹¹ to about 8×10¹³ vg/kg or about 1×10¹² to about 8×10¹³ vg/kg. In an aspect, a disclosed vector can be administered at a dose of about 1×10¹³ to about 6×10¹³ vg/kg. In an aspect, a disclosed vector can be administered at a dose of at least about 1×10¹⁰, at least about 5×10¹⁰, at least about 1×10¹¹, at least about 5×10¹¹, at least about 1×10¹², at least about 5×10¹², at least about 1×10¹³, at least about 5×10¹³, or at least about 1×10¹⁴ vg/kg. In an aspect, a disclosed vector can be administered at a dose of no more than about 1×10¹⁰, no more than about 5×10¹⁰, no more than about 1×10¹¹, no more than about 5×10¹¹, no more than about 1×10¹², no more than about 5×10¹², no more than about 1×10¹³, no more than about 5×10¹³, or no more than about 1×10¹⁴ vg/kg. In an aspect, a disclosed vector can be administered at a dose of about 1×10¹² vg/kg. In an aspect, a disclosed vector can be administered at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method of repairing a defective gene can comprise administering to the subject one or more additional therapeutic agents. In an aspect, a disclosed therapeutic agent can comprise enzyme replacement therapy, gene therapy, mRNA therapy, small molecule therapy, substrate reduction therapy, or any combination thereof.

In an aspect, a disclosed method of repairing a defective gene can comprise treating the subject. In an aspect, treating the subject can comprise administering to the subject one or more agents that modulate the level of one or more differentially present cellular metabolites. In an aspect, treating the subject can comprise implementing a change in the subject's dietary intake of carbohydrates. Implementing a change in the subject's dietary intake of carbohydrates can comprise adding carbohydrates to the subject's diet, or removing carbohydrates from the subject's diet, or changing the type of carbohydrates in the subject's diet, or changing the frequency of carbohydrates consumed by the subject. In an aspect, treating the subject can comprise administering cornstarch to the subject, or administering glycoside to the subject, or administering one or more anaplerotic agents to the subject.

In an aspect of a disclosed method of repairing a disclosed defective gene, a disclosed Cas9 nuclease can create a double-strand breaks (DSB) within or near the variant in the defective gene locus that results in a permanent integration of the repair template. In an aspect, a disclosed method of repairing a defective gene can improve the efficiency of gene editing. In an aspect, a disclosed method can be used to stably integrate a transgene into one or more disclosed cells. In an aspect, a disclosed method can improve the efficiency of gene editing.

In an aspect, a disclosed method of repairing a defective gene can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria. Such methods are known to the skilled person. In an aspect, a disclosed method can further comprise repeating a monitoring step.

In an aspect, a disclosed method of repairing a defective gene can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of repairing a defective gene can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of repairing a defective gene can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of repairing a defective gene can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method of repairing a defective GAA gene can further comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method of repairing a defective gene can further comprise administering a β2 agonist. For example, in an aspect, a disclosed method can comprises administering a β2 agonist to increase the expression of one or more receptors for a lysosomal enzyme. In an aspect, β2 agonists include but are not limited to albuterol, clenbuterol, formoterol, indacaterol, olodaterol, salmeterol, vilanterol, and any combination thereof, growth hormones (e.g., human growth hormone), autocrine glycoprotein (e.g., Follistatin), or any combination thereof (see, e.g., U.S. Pat. No. 8,679,478 for a discussion of appropriate β2 agonists, which patent is incorporated by reference it its entirety for these teachings).

In an aspect, a disclosed method of repairing a defective gene can further comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a disclosed method can further comprise diagnosing a subject with a genetic defect using one or more known methods to the skilled person, such as, for example, genotyping.

In an aspect of a disclosed method, contacting a cell can comprising methods known to the art. For example, contacting can comprise administering to a subject one or more disclosed compositions, disclosed isolated nucleic acid molecules, disclosed pharmaceutical formulations, and/or disclosed vectors.

In an aspect, administering can comprise intravenous, intraarterial, intramuscular, intraperitoneal, subcutaneous, intra-CSF, intrathecal, intraventricular, intrahepatic, hepatic intra-arterial, hepatic portal vein (HPV), or in utero administration. In an aspect, a disclosed composition, a disclosed isolated nucleic acid molecule, a disclosed pharmaceutical formulation, and/or a disclosed vector can be administered via intra-CSF administration in combination with RNAi, antisense oligonucleotides, miRNA, one or more small molecules, one or more therapeutic agents, one or more proteasome inhibitors, one or more immune modulators, and/or a gene editing system. In an aspect, a disclosed composition, a disclosed isolated nucleic acid molecule, a disclosed pharmaceutical formulation, and/or a disclosed vector can be administered via LNP administration. In an aspect, a disclosed composition, a disclosed isolated nucleic acid molecule, a disclosed pharmaceutical formulation, and/or a disclosed vector can be concurrently and/or serially administered to a subject via multiple routes of administration. For example, in an aspect, administering a disclosed nucleic acid molecule, a disclosed vector, and/or a disclosed pharmaceutical formulation can comprise intravenous administration and intra-cistern magna (ICM) administration. In an aspect, administering a disclosed composition, a disclosed isolated nucleic acid molecule, a disclosed pharmaceutical formulation, and/or a disclosed vector can comprise IV administration and intrathecal (ITH) administration. In an aspect, a disclosed method can employ multiple routes of administration to the subject. In an aspect, a disclosed method can employ a first route of administration that can be the same or different as a second and/or subsequent routes of administration.

In an aspect of a disclosed method of treating and/or preventing a genetic disease or disorder, a therapeutically effective amount of disclosed vector can be delivered to a subject via intravenous (IV) administration and can comprise a range of about 1×10¹⁰ vg/kg to about 2×10¹⁴ vg/kg. In an aspect, for example, a disclosed vector can be administered at a dose of about 1×10¹¹ vg/kg to about 8×10¹³ vg/kg or about 1×10¹² vg/kg to about 8×10¹³ vg/kg. In an aspect, a disclosed vector can be administered at a dose of about 1×10¹³ vg/kg to about 6×10¹³ vg/kg. In an aspect, a disclosed vector can be administered at a dose of at least about 1×10¹⁰ vg/kg, at least about 5×10¹⁰ vg/kg, at least about 1×10¹¹ vg/kg, at least about 5×10¹¹ vg/kg, at least about 1×10¹² vg/kg, at least about 5×10¹² vg/kg, at least about 1×10¹³ vg/kg, at least about 5×10¹³ vg/kg, or at least about 1×10¹⁴ vg/kg. In an aspect, a disclosed vector can be administered at a dose of no more than about 1×10¹⁰ vg/kg, no more than about 5×10¹⁰ vg/kg, no more than about 1×10¹¹ vg/kg, no more than about 5×10¹¹ vg/kg, no more than about 1×10¹² vg/kg, no more than about 5×10¹² vg/kg, no more than about 1×10¹³ vg/kg, no more than about 5×10¹³, or no more than about 1×10¹⁴ vg/kg. In an aspect, a disclosed vector can be administered to a subject at a dose of about 1×10¹² vg/kg. In an aspect, a disclosed vector can be administered to a subject at a dose of about 1×10¹¹ vg/kg. In an aspect, a disclosed vector can be administered in a single dose, or in multiple doses (such as 2, 3, 4, 5, 6, 7, 8, 9 or 10 doses) as needed for the desired therapeutic results.

In an aspect, a disclosed method of treating and/or preventing a genetic disease or disorder can further comprise administering to the subject a therapeutically effective amount of a therapeutic agent. A therapeutic agent can be any disclosed agent that effects a desired clinical outcome.

In an aspect, a disclosed method of treating and/or preventing a genetic disease or disorder can further comprise monitoring the subject for adverse effects. In an aspect, in the absence of adverse effects, the method can further comprise continuing to treat the subject. In an aspect, in the presence of adverse effects, the method can further comprise modifying the treating step. Methods of monitoring a subject's well-being can include both subjective and objective criteria (and are discussed supra). Such methods are known to the skilled person.

In an aspect, a disclosed method can further comprise administering to the subject a therapeutically effective amount of an agent that can correct one or more aspects of a dysregulated metabolic or enzymatic pathway. In an aspect, such an agent can comprise an enzyme for enzyme replacement therapy. In an aspect, a disclosed enzyme can replace any enzyme in a dysregulated or dysfunctional metabolic or enzymatic pathway. In an aspect, a disclosed method can comprise replacing one or more enzymes in a dysregulated or dysfunctional metabolic pathway.

In an aspect, a disclosed method of treating and/or preventing a genetic disease or disorder can further comprise administering one or more immune modulators. In an aspect, a disclosed immune modulator can be methotrexate, rituximab, intravenous gamma globulin, or bortezomib, or a combination thereof. In an aspect, a disclosed immune modulator can be bortezomib or SVP-Rapamycin. In an aspect, a disclosed immune modulator can be Tacrolimus. In an aspect, a disclosed immune modulator such as methotrexate can be administered at a transient low to high dose. In an aspect, a disclosed immune modulator can be administered at a dose of about 0.1 mg/kg body weight to about 0.6 mg/kg body weight. In an aspect, a disclosed immune modulator can be administered at a dose of about 0.4 mg/kg body weight. In an aspect, a disclosed immune modulator can be administered at about a daily dose of 0.4 mg/kg body weight for 3 to 5 or greater cycles, with up to three days per cycle. In an aspect, a disclosed immune modulator can be administered at about a daily dose of 0.4 mg/kg body weight for a minimum of 3 cycles, with three days per cycle. In an aspect, a person skilled in the art can determine the appropriate number of cycles. In an aspect, a disclosed immune modulator can be administered as many times as necessary to achieve a desired clinical effect.

In an aspect, a disclosed immune modulator can be administered orally about one hour before a disclosed therapeutic agent. In an aspect, a disclosed immune modulator can be administered subcutaneously about 15 minutes before a disclosed therapeutic agent. In an aspect, a disclosed immune modulator can be administered concurrently with a disclosed therapeutic agent. In an aspect, a disclosed immune modulator can be administered orally about one hour or a few days before a disclosed isolated nucleic acid molecule, a disclosed vector, a disclosed pharmaceutical formulation, or a combination thereof. In an aspect, a disclosed immune modulator can be administered subcutaneously about 15 minutes before or a few days before a disclosed isolated nucleic acid molecule, a disclosed vector, a disclosed pharmaceutical formulation, or a combination thereof. In an aspect, a disclosed immune modulator can be administered concurrently with a disclosed isolated nucleic acid molecule, a disclosed vector, a disclosed pharmaceutical formulation, or a combination thereof.

In an aspect, a disclosed method of treating and/or preventing a genetic disease or disorder can further comprise administering one or more proteasome inhibitors (e.g., bortezomib, carfilzomib, marizomib, ixazomib, and oprozomib). In an aspect, a proteasome inhibitor can be an agent that acts on plasma cells (e.g., daratumumab). In an aspect, an agent that acts on a plasma cell can be melphalan hydrochloride, melphalan, pamidronate disodium, carmustine, carfilzomib, carmustine, cyclophosphamide, daratumumab, doxorubicin hydrochloride liposome, doxorubicin hydrochloride liposome, elotuzumab, melphalan hydrochloride, panobinostat, ixazomib citrate, carfilzomib, lenalidomide, melphalan, melphalan hydrochloride, plerixafor, ixazomib citrate, pamidronate disodium, panobinostat, plerixafor, pomalidomide, pomalidomide, lenalidomide, selinexor, thalidomide, thalidomide, bortezomib, selinexor, zoledronic acid, or zoledronic acid.

In an aspect, a disclosed method of improving transgene stability can further comprise administering one or more proteasome inhibitors or agents that act on plasma cells prior to administering a disclosed isolated nucleic acid molecule, a disclosed vector, or a disclosed pharmaceutical formulation. In an aspect, a disclosed method can comprise administering one or more proteasome inhibitors or one or more agents that act on plasma cells concurrently with administering a disclosed isolated nucleic acid molecule, a disclosed vector, or a disclosed pharmaceutical formulation. In an aspect, a disclosed method can comprise administering one or more proteasome inhibitors or one or more agents that act on plasma cells subsequent to administering a disclosed isolated nucleic acid molecule, a disclosed vector, or a disclosed pharmaceutical formulation. In an aspect, a disclosed method can further comprise administering one or more proteasome inhibitors more than 1 time. In an aspect, a disclosed method can comprise administering one or more proteasome inhibitors repeatedly over time.

In an aspect, a disclosed method of treating and/or preventing a genetic disease or disorder can further comprise administering one or more immunosuppressive agents. In an aspect, an immunosuppressive agent can be, but is not limited to, azathioprine, methotrexate, sirolimus, anti-thymocyte globulin (ATG), cyclosporine (CSP), mycophenolate mofetil (MMF), steroids, or a combination thereof. In an aspect, a disclosed method can comprise administering one or more immunosuppressive agents more than 1 time. In an aspect, a disclosed method can comprise administering one or more one or more immunosuppressive agents repeatedly over time. In an aspect, a disclosed method can comprise administering a compound that targets or alters antigen presentation or humoral or cell mediated or innate immune responses.

In an aspect, a disclosed method of treating and/or preventing a genetic disease or disorder can further comprise administering a compound that exerts a therapeutic effect against B cells and/or a compound that targets or alters antigen presentation or humoral or cell mediated immune response. In an aspect, a disclosed compound can be rituximab, methotrexate, intravenous gamma globulin, anti CD4 antibody, anti CD2, an anti-FcRN antibody, a BTK inhibitor, an anti-IGF1R antibody, a CD19 antibody (e.g., inebilizumab), an anti-IL6 antibody (e.g., tocilizumab), an antibody to CD40, an IL2 mutein, or a combination thereof. Also disclosed herein are Treg infusions that can be administered as a way to help with immune tolerance (e.g., antigen specific Treg cells to AAV).

In an aspect, a disclosed method of treating and/or preventing a genetic disease or disorder can further comprise treating a subject that has developed or is likely to develop neutralizing antibodies (ABs) to a disclosed vector, a disclosed capsid, and/or a disclosed transgene. In an aspect, treating a subject that has developed or is likely to develop neutralizing antibodies can comprise plasmapheresis and immunosuppression. In an aspect, a disclosed method can comprise using immunosuppression to decrease the T cell, B cell, and/or plasma cell population, decrease the innate immune response, inflammatory response, and antibody levels in general. In an aspect, a disclosed method can comprise administering an IgG-degrading agent that depletes pre-existing neutralizing antibodies. In an aspect, a disclosed method can comprise administering to the subject IdeS or IdeZ, rapamycin, and/or SVP-Rapamycin. In an aspect, a disclosed method can comprise administering Tacrolimus. In an aspect, a disclosed IgG-degrading agent is bacteria-derived IdeS or IdeZ.

In an aspect, a disclosed method can comprise repeating a disclosed administering step such as, for example, repeating the administering of a disclosed isolated nucleic acid molecule, a disclosed vector, a disclosed pharmaceutical formulation, a disclosed therapeutic agent, a disclosed immune modulator, a disclosed proteasome inhibitor, a disclosed immunosuppressive agent, a disclosed compound that exerts a therapeutic effect against B cells and/or a disclosed compound that targets or alters antigen presentation or humoral or cell mediated immune response.

In an aspect, a disclosed method can further comprise administering a β2 agonist. For example, in an aspect, a disclosed method can comprises administering a β2 agonist to increase the expression of one or more receptors for a lysosomal enzyme. In an aspect, β2 agonists include but are not limited to albuterol, clenbuterol, formoterol, indacaterol, olodaterol, salmeterol, vilanterol, and any combination thereof, growth hormones (e.g., human growth hormone), autocrine glycoprotein (e.g., Follistatin), or any combination thereof (see, e.g., U.S. Pat. No. 8,679,478 for a discussion of appropriate β2 agonists, which patent is incorporated by reference it its entirety for these teachings).

In an aspect, a disclosed method can comprise modifying one or more of the disclosed steps. For example, modifying one or more of steps of a disclosed method can comprise modifying or changing one or more features or aspects of one or more steps of a disclosed method. For example, in an aspect, a method can be altered by changing the amount of one or more of the disclosed isolated nucleic acid molecules, disclosed vectors, disclosed pharmaceutical formulations, or a combination thereof administered to a subject, or by changing the frequency of administration of one or more of the disclosed isolated nucleic acid molecules, disclosed vectors, disclosed pharmaceutical formulations, or a combination thereof to a subject, or by changing the duration of time one or more of the disclosed isolated nucleic acid molecules, disclosed vectors, disclosed pharmaceutical formulations, or a combination are administered to a subject.

In an aspect, a method can be altered by changing the amount of one or more disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject, or by changing the frequency of administration of one or more of the disclosed therapeutic agents, disclosed immune modulators, disclosed proteasome inhibitors, disclosed immunosuppressive agents, disclosed compounds that exert therapeutic effect against B cells and/or disclosed compounds that targets or alters antigen presentation or humoral or cell mediated immune response administered to a subject.

In as aspect, a disclosed method can comprise concurrent administration of one or more of the following: one or more disclosed isolated nucleic acid molecules, one or more disclosed vectors, one or more disclosed pharmaceutical formulations, one or more disclosed therapeutic agents, one or more disclosed immune modulators, one or more disclosed proteasome inhibitors, one or more disclosed immunosuppressive agents, one or more disclosed compounds that exert therapeutic effect against B cells, one or more disclosed compounds that targets or alters antigen presentation or humoral or cell mediated immune response, or any combination thereof.

In an aspect, a disclosed immune modulator can be administered prior to or after the administration of a disclosed therapeutic agent.

In an aspect, one or more isolated nucleic acid molecules or one or more vectors can be administered concurrently or sequentially.

In an aspect, a disclosed method of treating and/or preventing a genetic disease or disorder can further comprise generating a disclosed isolated nucleic acid molecule. In an aspect, a disclosed method can further comprise generating a disclosed viral or non-viral vector. In an aspect, generating a disclosed viral vector can comprise generating an AAV vector or a recombinant AAV (such as those disclosed herein). In an aspect, a disclosed method can further comprise gene editing one or more relevant genes (such as, for example, a missing, deficient, and/or mutant protein or enzyme), wherein editing includes but is not limited to single gene knockout, loss of function screening of multiple genes at one, gene knockin, or a combination thereof.

### G. Kits

Disclosed herein is a kit comprising a disclosed isolated nucleic acid molecule, a disclosed vector, a disclosed pharmaceutical formulation, or any combination thereof. Disclosed herein is a kit comprising one or more disclosed isolated nucleic acid molecules, one or more disclosed vectors, one or more disclosed pharmaceutical formulations, or any combination thereof. In an aspect, a kit can comprise a disclosed isolated nucleic acid molecule, a disclosed vector, a disclosed pharmaceutical formulation, or a combination thereof, and one or more agents. “Agents” and “Therapeutic Agents” are known to the art and are described supra.

In an aspect, the one or more agents can treat, prevent, inhibit, and/or ameliorate one or more comorbidities in a subject. In an aspect, one or more active agents can treat, inhibit, prevent, and/or ameliorate cellular and/or metabolic complications related to a missing, deficient, and/or mutant protein or enzyme (such as, for example, GAA).

In an aspect, a disclosed kit can comprise at least two components constituting the kit. Together, the components constitute a functional unit for a given purpose (such as, for example, treating a subject diagnosed with or suspected of having a disease or disorder). Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation. In an aspect, a kit for use in a disclosed method can comprise one or more containers holding a disclosed isolated nucleic acid molecule, a disclosed vector, a disclosed pharmaceutical formulation, or a combination thereof, and a label or package insert with instructions for use. In an aspect, suitable containers include, for example, bottles, vials, syringes, blister pack, etc. The containers can be formed from a variety of materials such as glass or plastic. The container can hold a disclosed isolated nucleic acid molecule, a disclosed vector, a disclosed pharmaceutical formulation, or a combination thereof, and can have a sterile access port (for example the container may be an intravenous solution bag or a vial having a stopper pierceable by a hypodermic injection needle). The label or package insert can indicate that a disclosed isolated nucleic acid molecule, a disclosed vector, a disclosed pharmaceutical formulation, or a combination thereof can be used for treating, preventing, inhibiting, and/or ameliorating a disease or disorder or complications and/or symptoms associated with a disease or disorder. A kit can comprise additional components necessary for administration such as, e.g., other buffers, diluents, filters, needles, and syringes.

In an aspect, a disclosed kit can be used to repair a defective GAA gene or any genetic defect. In an aspect, a disclosed kit can be used to treat a subject having Pompe disease. In an aspect, a disclosed kit can be used to validating the efficacy of a gene editing system. In an aspect, a disclosed kit can be used to treat and/or prevent a genetic disease or disorder.

### EXAMPLES

Gene therapy to replace GAA with AAV vectors is expected to be less effective early in life due to the rapid loss of vector genomes during infancy. One of the main obstacles to the development of gene therapy targeted to the liver is the gradual loss of episomal AAV vector genomes, which is accelerated early in life (Cunningham S C, et al. (2008) Mol Ther. 16:1081-1088; Cunningham S C, et al. (2009) Mol Ther. 17:1340-1346; Wang L, et al. (2011) Mol Ther. 19:2012-2020; Lee E K, et al. (2012) Mol Ther. 20:1844-1851). Although AAV vectors have advanced to successful clinical trials based upon liver transgene expression (Nathwani A C, et al. (2014) N Engl J Med. 371:1994-2004), the loss of vector genomes has exceeded the rate expected solely from cell division in the liver (Cunningham S C, et al. (2008) Mol Ther. 16:1081-1088; Wang L, et al. (2011) Mol Ther. 19:2012-2020). Approaches to this problem have included higher vector dosages (Cunningham S C, et al. (2009) Mol Ther. 17:1340-1346; Cotugno G, et al. (2011) Mol Ther. 19:461-469), and early re-administration of the vector, prior to the formation of anti-AAV antibodies (Lee E K, et al. (2012) Mol Ther. 20:1844-1851). These approaches have not comprehensively addressed the negative effects on efficacy of the loss of vector genomes in animal models for genetic disease following neonatal treatment. However, the long-term benefits of gene therapy in infant mice with Pompe disease confirm the potential value of treatment early in life (Mah C, et al. (2005) Gene Ther. 12:1405-1409; Mah C, et al. (2007) Mol Ther. 15:501-507; Han S O, et al. (2020) Mol Ther Methods Clin Dev. 17:133-142).

Preclinical data have suggested the early treatment with gene therapy might be successful in Pompe disease. The long-term efficacy of liver depot gene therapy with AAV2/8-LSPhGAA was performed and directly compared the efficacy of a clinically appropriate dose of an rAAV8 vector in infant and adult GAA-KO mice (Han S O, et al. (2020) Mol Ther Methods Clin Dev. 17:133-142). AAV2/8-LSPhGAA (3×10¹⁰ vg/mouse) was administered to infant (10 day old) or adult (2-month-old) GAA knockout mice. Biochemical correction and muscle function were evaluated 50 weeks following intravenous administration of the same absolute vector dosages at 10 days or 2 months of age to assess the effects of gene therapy either early or later in life. Unsurprisingly, the degree of biochemical correction was greater in the adult-treated mice, which was due to the fact that AAV vector transduction was more stable in older animals that have completed the rapid growth phase of infancy. Improved wire hang test latency was observed for treated adults (<0.05), but not for infant mice. The relative vector dose for infants was approximately 3-fold higher than adults, when normalized to body weight at the time of vector administration (Han S O, et al. (2020) Mol Ther Methods Clin Dev. 17:133-142). Given these data, the dose requirement to achieve similar efficacy will be higher for the treatment of young patients, and the benefits from treatment early in life will be less from gene replacement. Genome editing has potential to stably transduce hepatocytes to improve the response to therapy early in life.

Single AAV vectors for genome editing are being developed using the small Cas9 from Staphylococcus aureus (SaCas9), which can be packaged along with a single guide RNA (sgRNA) and repair template in a single AAV vector for genome editing (Friedland A E, et al. (2015) Genome Biol. 16:257; Ran F A, et al. (2015) Nature. 520:186-191). This single vector strategy will increase the efficiency of gene correction by eliminating the need for transduction with a second vector to achieve genome editing. The relatively low efficiency of AAV transduction in skeletal muscle (<1 vector genome per nucleus) confirms the need for a single vector system to deliver CRISPR-Cas9 more efficiently during genome editing (Chen S J, et al. (2013) Hum Gene Ther Clin Dev. 24:154-160; Yi H, et al. (2017) Hum Gene Ther. 28:286-294). A recent study demonstrated that an rAAV9 vector transduced and edited satellite cells, as well as myocytes, to achieve a renewable population of corrected cells in muscle, increasing the stability of editing in skeletal muscle (Nance M E, et al. (2019) Mol Ther. 27:1568-1585). The development of single rAAV9 vector genome editing for muscle cells, including satellite cells, will stably correct Pompe disease with implications for inherited myopathies and muscular dystrophies.

This study sought to demonstrate the correction of pathogenic variants that cause Pompe disease in muscle, developing a therapeutic strategy to benefit patients with Pompe disease and potentially other muscle diseases (Smith E C, et al. (2011) Rheum Dis Clin North Am. 37:201-217). Vectors were developed to correct two common GAA variants that occur in the majority of patients with Pompe disease: (i) the c.-32-13T->G variant that is present in ˜90% of patients with late-onset Pompe disease, commonly known as the “IVS1” variant (45% of all variants) (Kroos M A, et al. (2007) Neurology. 68:110-115; Reuser A J, et al. (2019b) Hum Mutat. 40:2146-2164a); and (ii) the c.525delT (ΔT525) variant that is associated with infantile-onset Pompe disease and is common among Dutch patients (6% of all variants) (Van der K M, et al. (1994) Biochem Biophys Res Commun. 203:1535-1541). These variants can be corrected with genome editing to restore function of one variant on one allele, because Pompe disease is an autosomal recessive disorder and carriers have no known symptoms.

### Example 1

### Validation of Genome Editing at the IVS1 and ΔT525 to Correct Point Mutations

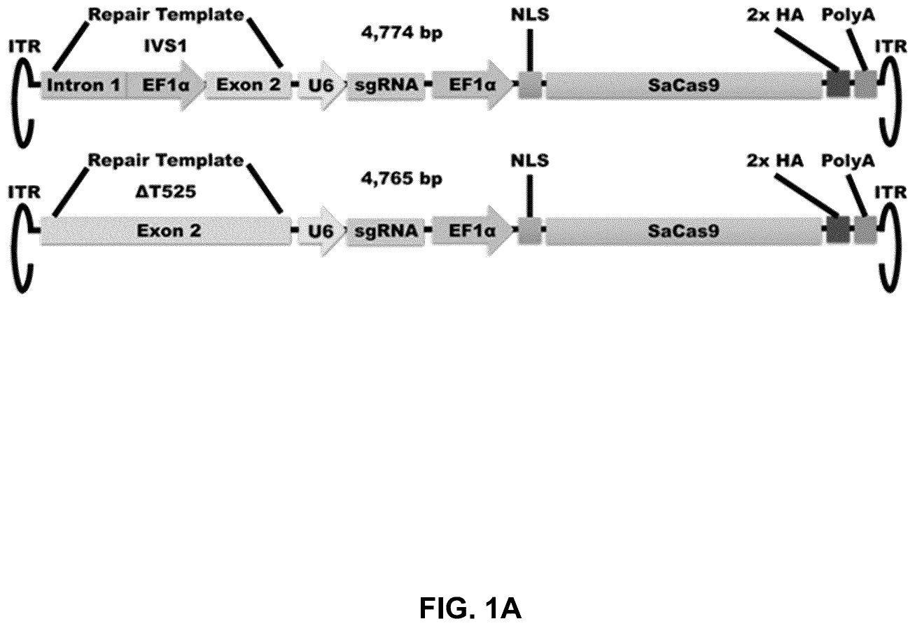

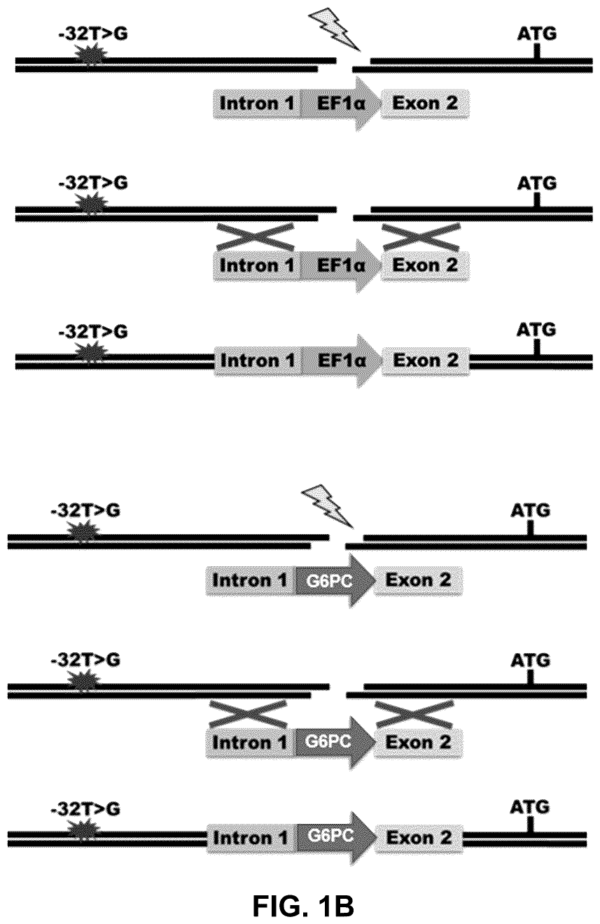

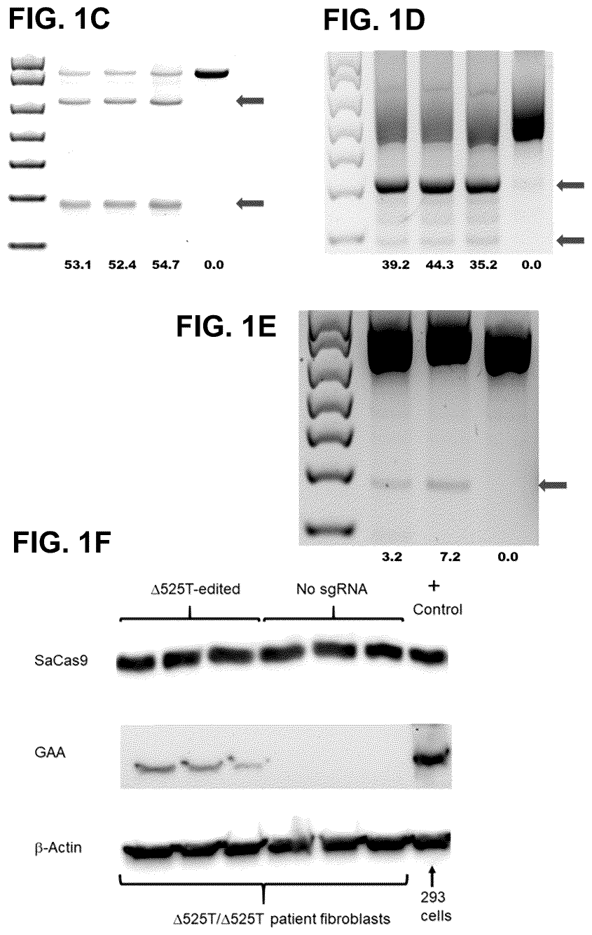

A CRISPR/Cas9 cassette that cleaved at the IVS1 variant near the intron 1/exon 2 boundary in the GAA gene was designed. (FIG. 1A). Three (3) protospacer adjacent motif (PAM) sequences near the IVS1 variant where SaCas9 will cleave were identified, and the corresponding sgRNAs to guide SaCas9 to the PAMs were incorporated into an AAV vector plasmid to achieve genome editing at that site. The sgRNA that achieved the highest rate of cutting was incorporated into a 4,774 bp AAV-CRISPR vector. (FIG. 1A). The vector contains a 650 bp repair template, including two small 215 bp homology arms derived from intron 1 and exon 2, which flank an EF1a promoter. The integrated EF1a promoter will drive high level GAA expression due to the position of the translational start site in exon 2 of the GAA gene. (FIG. 1A). The vector plasmid was transfected into Pompe disease patient fibroblasts, achieving HDR-mediated correction in 52% of alleles. (FIG. 1B). Furthermore, transduction of human induced pluripotent stem cell (hiPSC)-derived cardiomyocytes with this rAAV9-CRISPR vector induced HDR to >7%. (FIG. 1D). These data show the ability to initiate Cas9-mediated cleavage and donor template-mediated HDR using 215 bp homology arms. Similarly, two SaCas9 PAM sites near the ΔT525 variant in exon 2 of GAA were identified, and one of these has been incorporated into a 4,765 bp AAV-CRISPR vector. (FIG. 1A). The vector plasmid was transfected into Pompe disease patient fibroblasts, cleaving the target DNA and incorporating a 600 bp repair template, and introducing a BamHI restriction enzyme recognition site in >52% of alleles. (FIG. 1D). The repair template contains a mutated PAM site to prevent re-cleavage of the corrected gene. These data demonstrated the ability to initiate genome editing at multiple distinct variants in GAA, thereby achieving “personalized genome editing”.

### Example 2

### Use of a Muscle-Active Promoter to Advantageously Correct a Duplication

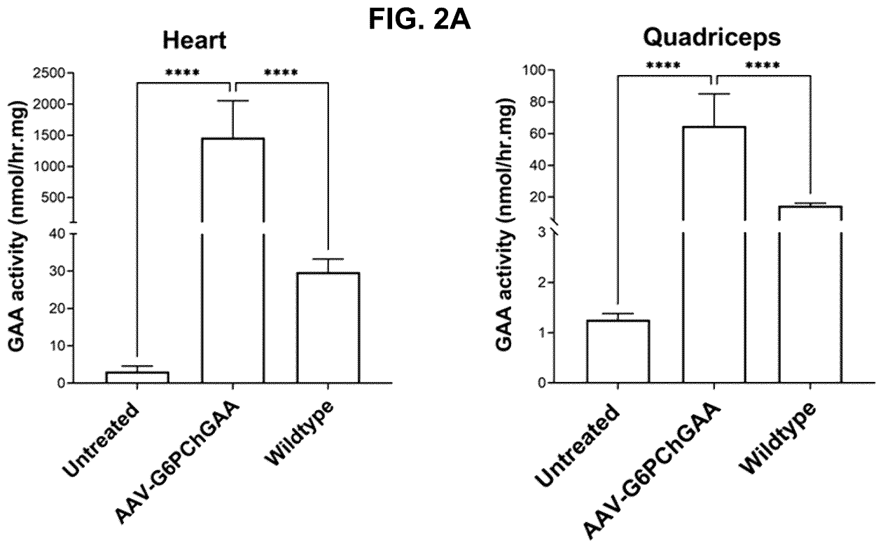

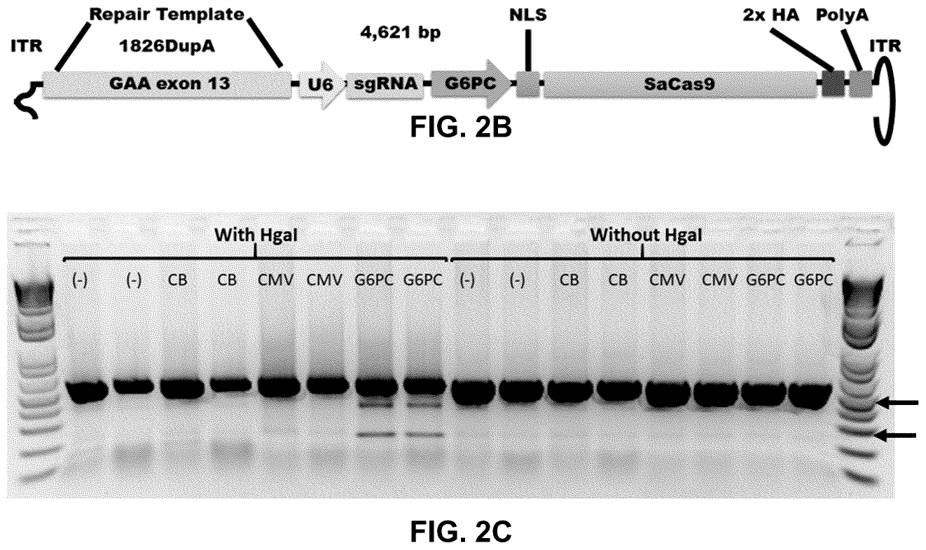

The human G6PC minimal 298 bp promoter had high activity in the heart and skeletal muscle when a transgene containing the G6PC minimal 298 bp promoter to drive human GAA expression was delivered by an AAV9 vector in GAA knockout mice with Pompe disease. (FIG. 2A). This was surprising because (i) this G6PC minimal promoter sequence previously demonstrated its ability to drive expression in hepatoma cells and (ii) it contains HNF1 and HNF5 sites associated with liver specific expression (Schmoll D, et al. (1996) FEBS Letters. 383:63-66). G6PC is primarily expressed in liver and kidneys, and bi-allelic pathogenic variants in G6PC cause glycogen storage disease type Ia (GSD Ia) that is characterized by glycogen accumulation in liver and kidney (Chen Y T, et al. (2001) Glycogen Storage Diseases, pp. 1521-1551 in The Metabolic and Molecular Bases of Inherited Disease, edited by C. R. Scriver, A. L. Beaudet, W. S. Sly and D. Valle. McGraw-Hill, New York). The activation of the G6PC minimal 298 bp promoter is based on the presence of “sequence from −161 to −152 [that] is a cAMP response element which is important for the regulation of transcription of the glucose 6-phosphatase gene by both cAMP and glucocorticoids” (Schmoll D, et al. (1999) Biochem J. 338:457-463). The absence of upstream sequences in the G6PC minimal 298 bp promoter helps to explain its activity in striated muscle. Given the demonstration that the G6PC minimal 298 bp promoter was active in liver and kidney (Luo X, et al. (2011) Mol Ther. 19:1961-1970), the G6PC minimal 298 bp promoter was expected to be active in liver, kidney, and striated muscle, at least. Therefore, the G6PC minimal 298 bp promoter represented a very small and highly muscle-active promoter that was optimized for transgene expression in the context of gene replacement therapy and genome editing in genetic diseases and other applications.

The ability of the (6PC minimal 298 bp promoter to drive Cas9 expression in the context of genome editing at a unique mutation causing Pompe disease (1826DupA) was evaluated. (FIG. 2B). The vector containing the (6PC promoter achieved the integration of the repair template through HDR whereas neither an analogous vector containing the CB promoter or a CMV promoter generated HDR. (FIG. 2C). These data support the ability of a single AAV vector containing a highly muscle active (6PC minimal promoter to express Cas9 to perform genome editing.

### Example 3

### Genome Editing to Create a GAA Depot

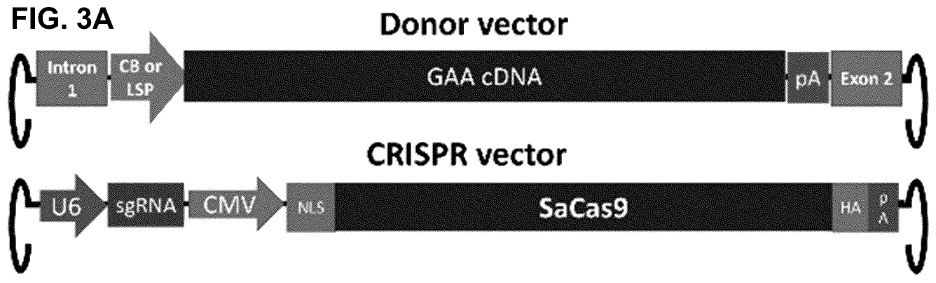

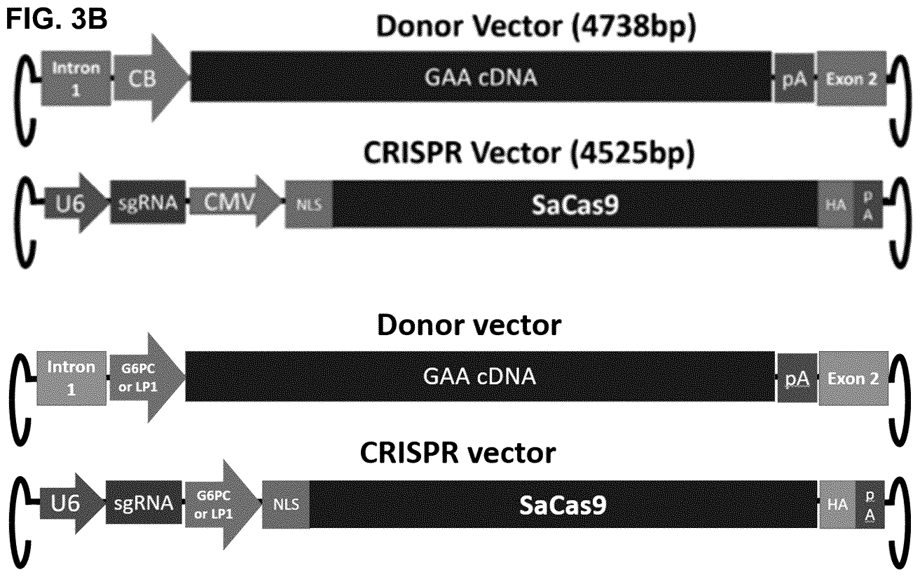

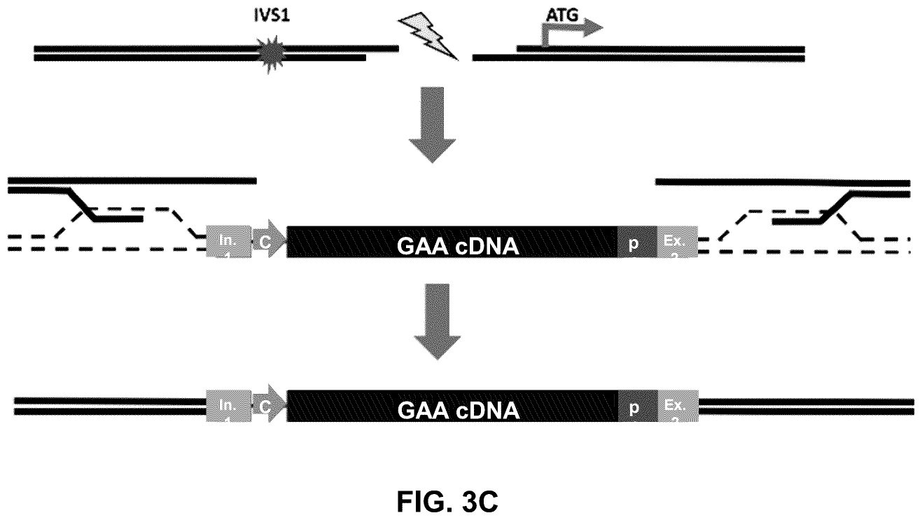

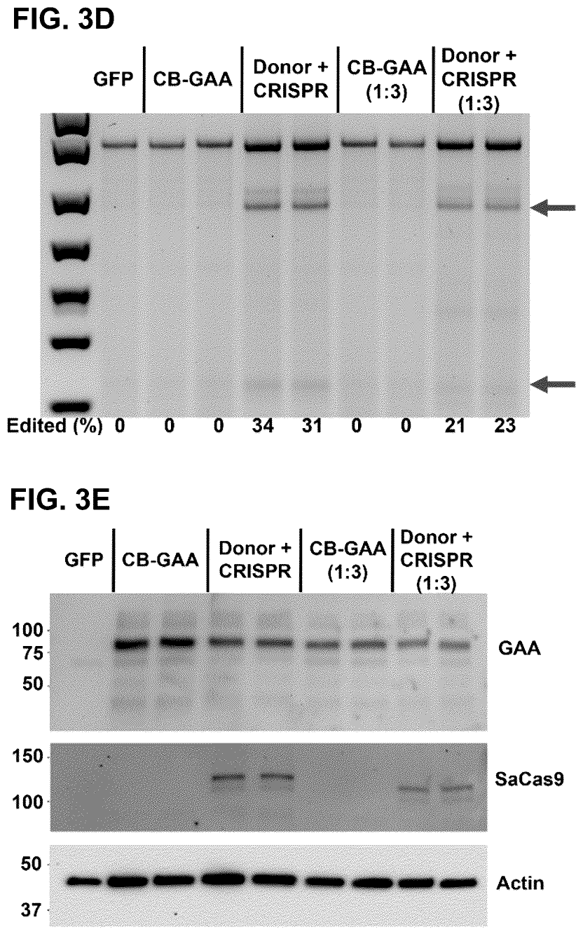

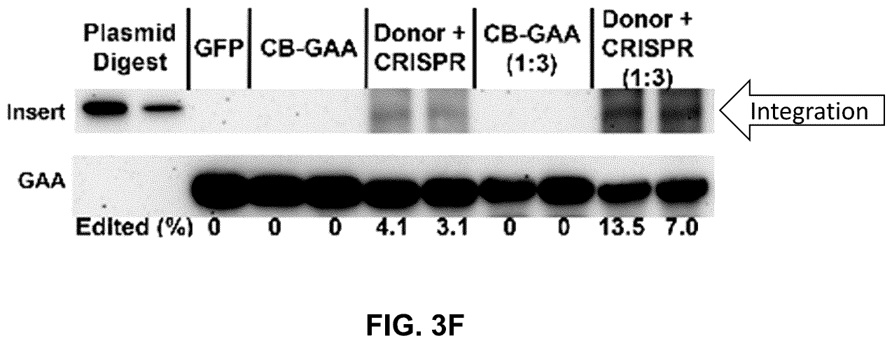

Very small 215 bp GAA homology arms targeted the IVS1 variant to insert an EF1a promoter through HDR, demonstrating the insertion of a heterologous sequence at the intron 1/exon 2 boundary. (FIG. 1A and FIG. 1B). This design was adapted to insert a high-activity transgene containing GAA based upon previous efficacious AAV vectors. (FIG. 3A). In this experiment, a second AAV vector delivered CRISPR/Cas9 to cleave near the IVS1 variant, upstream of the start codon. (FIG. 3B). The transgene was integrated through HDR to drive GAA expression, thereby creating a stable depot for GAA in the edited cells. Transfected patient fibroblasts were used to evaluate editing with these vectors. The CRISPR vector generated indels through non-homologous end joining (NHEJ) detected by the Surveyor assay, which demonstrated nuclease activity at the PAM site near IVS1. (FIG. 3C). The nuclease introduced double-stranded breaks leading to HDR-mediated transgene integration. (FIG. 3D). Cells containing integrated transgene expressed GAA and Cas9. Cell division slightly diluted GAA activity, which was consistent with the presence of unintegrated transgene, but following the Donor+CRISPR treatment, the transgene expression appeared to be more stable. (FIG. 3E). These data supported the ability to integrate a GAA transgene with genome editing to create a depot for GAA, which would treat Pompe disease regardless of the underlying variant and represents “universal genome editing”.

### Example 4

### Demonstration of Liver Depot Editing

Two constructs (SEQ ID NO:08 and SEQ ID NO:09) are tested in mice for the ability to demonstrate liver depot editing. The expression constructs are delivered by an AAV9 vector in GAA knockout mice with Pompe disease. These experiments demonstrate the feasibility of inserting an entire, functional transgene into a targeted region of the genome (i.e., the GAA cDNA into the endogenous mouse GAA locus). The resulting editing occurs primarily occur in liver (due to the liver promoter). GAA is expressed in the liver, thereby creating the depot that services to provide GAA to other tissues through blood.

### Example 5

### Generation of Constructs

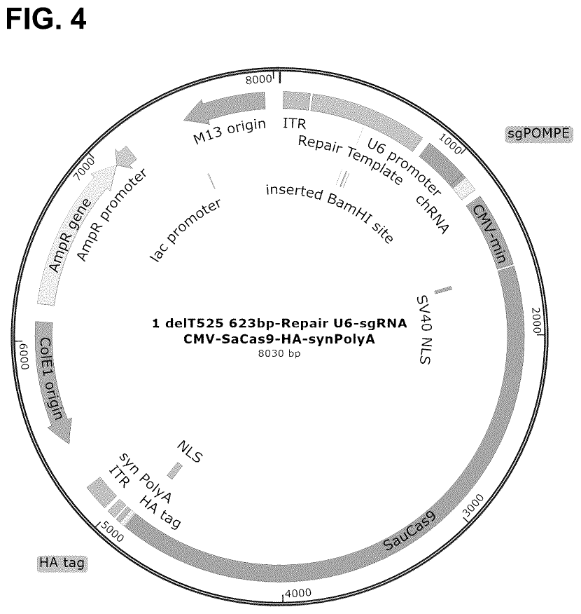

Construct 1 is titled “delT525 623 bp-Repair U6-sgRNA CMV-SaCas9-HA-synPolyA”. (FIG. 4) This was the original single vector gene editing construct constructed for Pompe disease. The repair template (623 bp) contains the T residue to restore the reading frame of GAA (e.g., WT GAA sequence or WT GAA exon 2 sequence). This repair template (del.T525 repair template) also has silent mutations to remove each PAM (for guides B and C (worked better than B)) and introduce a BamHI restriction site (this is to detect integration in vitro). The sequence includes the sequence for the U6 promoter, the CRISPR/transactivating RNA (chRNA), the CMV promoter, the SaCas9, the NLS, the HA tags, and a synthetic PolyA. This construct worked well in HEK293T cells but its size made it difficult to package into AAV.

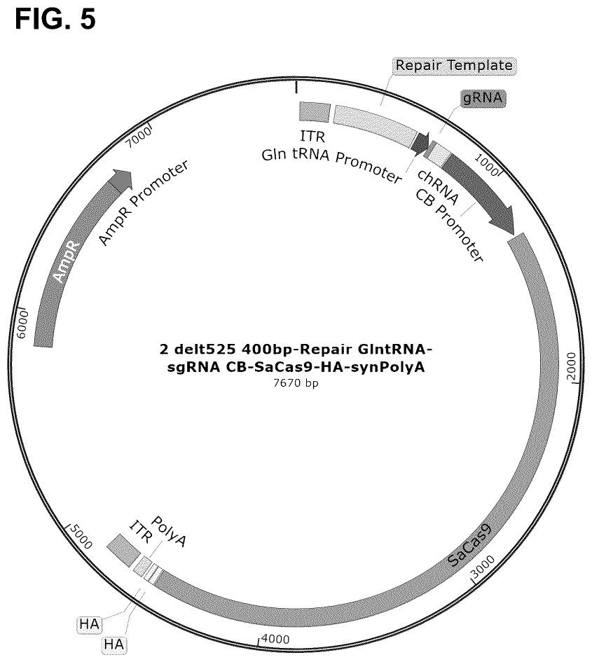

Construct 2 is titled “delT525 400 bp-Repair GlntRNA-sgRNA CB-SaCas9-HA-synPolyA”. (FIG. 5) This plasmid contains a smaller repair template (some of the 5′ and 3′ sequence was removed). The “center” of the repair template was the sgRNA PAM site, which left approximately 200 bp of homologous sequence on either side of the double stranded break in the genome. The repair template still contained the T at position 525 and silent mutations for the PAM and BamHI site. A glutamine tRNA sequence was used to express the sgRNA. Separately, a minimal CB promoter was used to try to achieve better expression in muscle. This vector was appropriate for genome editing across many tissues including the brain (using IV delivery of an AAV9 vector that would cross the blood-brain barrier at high dosages).

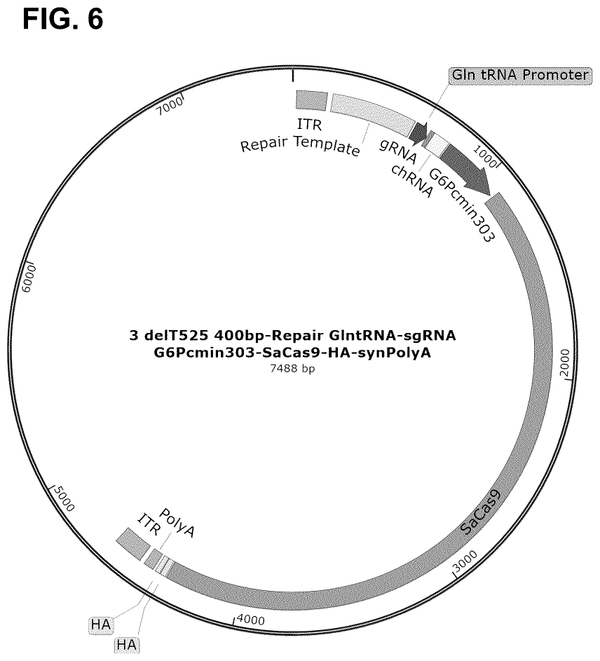

Construct 3 is titled “delT525 400 bp-Repair GlntRNA-sgRNA G6Pcmin303-SaCas9-HA-synPolyA”. (FIG. 6) Here, the 303 bp G6Pc minimal promoter replaced the CMV promoter. The 303 bp G6Pc minimal promoter previously worked well in mice so was employed here to achieve better expression in skeletal and cardiac muscle. This vector was appropriate for the editing of heart and skeletal muscle to treat muscle involvement in Pompe disease.

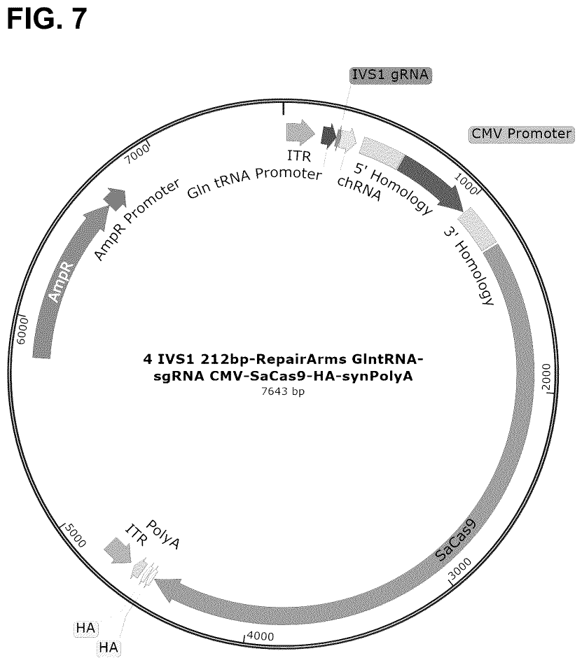

Construct 4 is titled “IVS1 212 bp-RepairArms GlntRNA-sgRNA CMV-SaCas9-HA-synPolyA”. (FIG. 7) Due to the limited size of AAV, the CMV promoter in the repair template also expressed Cas9. Here, integration with homology arms around 200 bp—around 215 bp each was achieved. This repair template was made and tested with 3 sgRNAs so the “center” of the template was in the middle of each of the 3 PAMs. The upstream homology arm was completely within intron 1. The downstream homology arm started in intron 1 and continued into exon 2. This sequence contained the best sgRNA. This vector was appropriate for use in genome editing across many tissues including the brain (using IV delivery of an AAV9 vector that would cross the blood-brain barrier at high dosages). Here, the more preferred promoter would be CB, LP1, or G6PC (instead of EF1a). An alternative vector can have a polyA signal upstream of the promoter to prevent read-through transcription from the endogenous GAA promoter. In an aspect, a polyA signal can be optional.

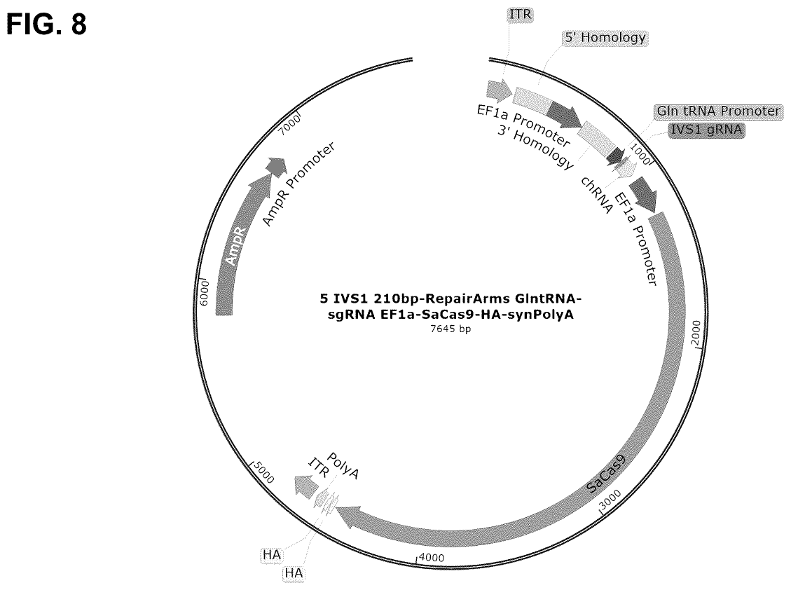

Construct 5 is titled “IVS1 210 bp-RepairArms GlntRNA-sgRNA EF1a-SaCas9-HA-synPolyA”. (FIG. 8) The plasmid had separate promoters in the repair template and expressing Cas9. The EF1a promoter was used because of its small size and ubiquitous expression. The homology arms for the repair template were nearly identical to the CMV IVS1 construct except that the homology arms were shifted about 30 bp downstream in the genome so that the “center” of the repair templet was directly over the PAM site for the best sgRNA. These homology arms in the IVS1 repair templates worked well. The template was used in the experiments represented in FIG. 1A-FIG. 21 and FIG. 1E (with AAV in human cardiomyocytes)

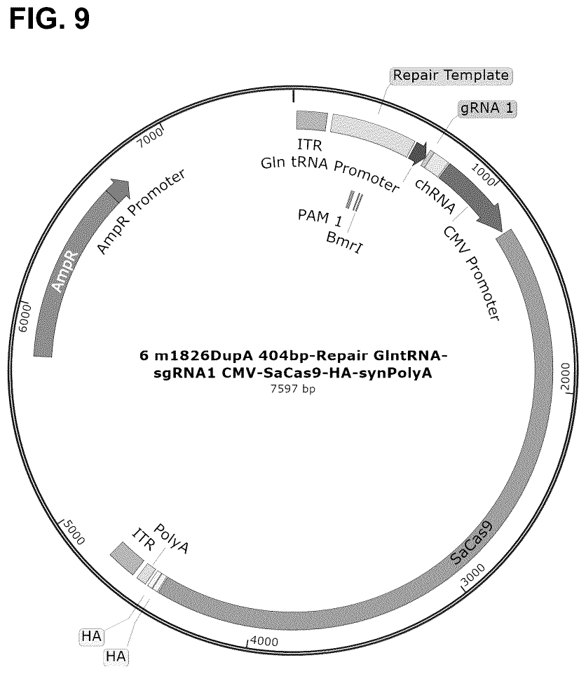

Construct 6 is titled “m1826DupA 404 bp-Repair GlntRNA-sgRNA1 CMV-SaCas9-HA-synPolyA” (FIG. 9). Plasmid 6 represents a single CRISPR vector for editing a mutation in mice. Another version contained a G6Pc minimal promoter expressing Cas9. Again there is about 200 bp of homology in the repair template to each side of the double stranded genomic DNA break. The data was represented in FIG. 3B; but it did not achieve editing.

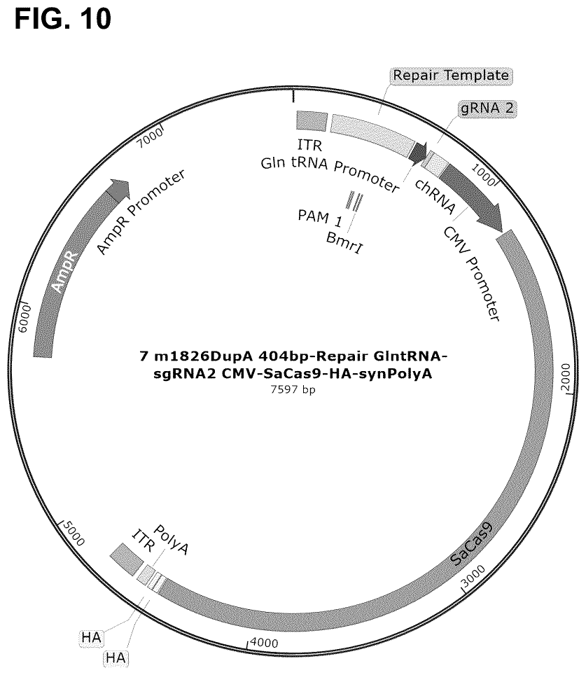

Construct 7 is titled “m1826DupA 404 bp-Repair GlntRNA-sgRNA2 CMV-SaCas9-HA-synPolyA”. (FIG. 10) This vector had a different sgRNA targeting the same mutation in mice. There is another version made with the G6Pc minimal promoter.

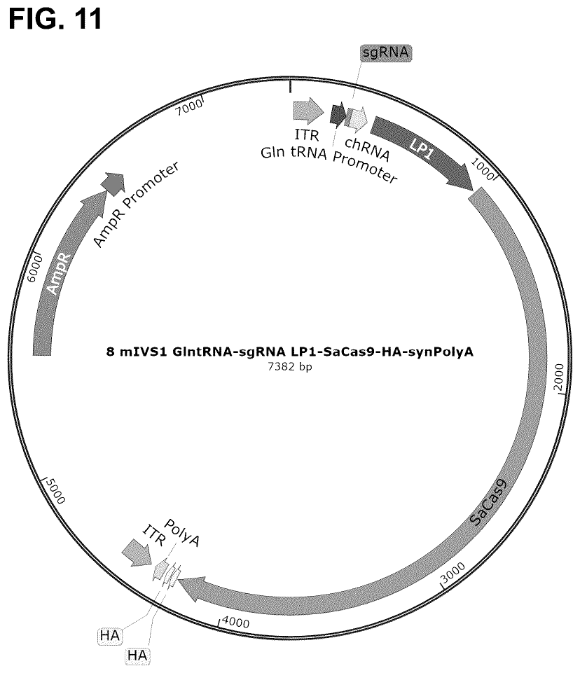

Construct 8 is titled “mIVS1 GlntRNA-sgRNA1 LP1-SaCas9-HA-synPolyA”. (FIG. 11) This construct contained the LP1 promoter (573 bp) sequence and the sgRNA for editing in mice. The same region of GAA in mice (corresponding to the human IVS1 editing) was targeted. The target sequence and PAM was just upstream of Exon 2, which would correct the IVS1 variant (most common), and thereby created a liver depot for GAA production. This was likely the best of all strategies in terms of feasibility and number of patients to be treated. The dose would be low for liver-targeted genome editing, which is critical to avoid immune responses that interfere with efficacy from gene therapy/editing with AAV vectors. This construct worked with vector 9 (described below).

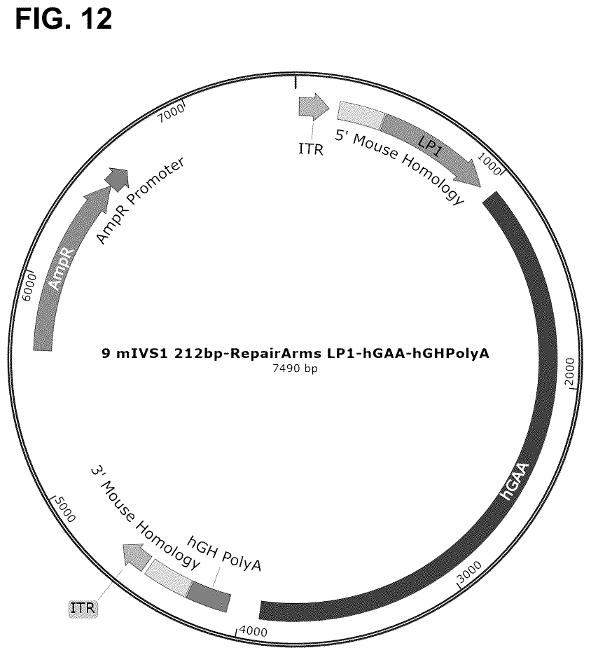

Construct 9 is titled “mIVS1 212 bp-RepairArms LP1-hGAA-hGHPolyA”. (FIG. 12) This construct contained the hGAA cDNA sequence as well as a shorter sequence (556 bp), which was due to the use of a restriction site at the 3′ end during cloning.

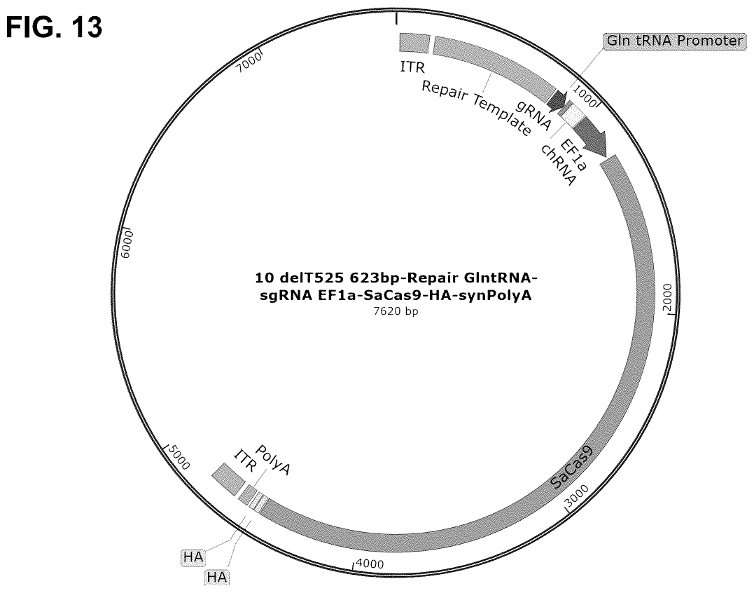

Construct 10 is titled “delT525 623 bp-Repair GlntRNA-sgRNA EF1a-SaCas9-HA-synPolyA”. Here, the EF1a promoter was used due to its small size and ubiquitous expression, features that allowed for packaging into AAV and transgene expression in liver and skeletal and cardiac muscle. This construct contained a 623 bp repair template to correct the ΔT525 mutation, the GlntRNA promoter to drive transcription of the sgRNA, and the EF1a promoter to drive expression of SaCas9. (FIG. 13)

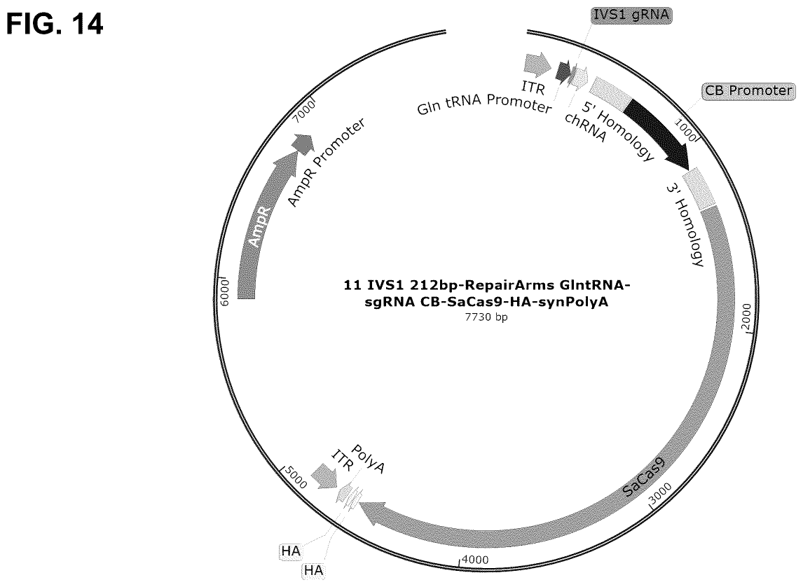

Construct 11 is titled “IVS1 212 bp-RepairArms GlntRNA-sgRNA CB-SaCas9-HA-synPolyA”. Here, this construct contained 212 bp repair arms flanking the CB promoter, which allow insertion of the CB promoter in intron 1. The CB promoter also expressed SaCas9 and provided stronger expression in skeletal and cardiac muscle. (FIG. 14)

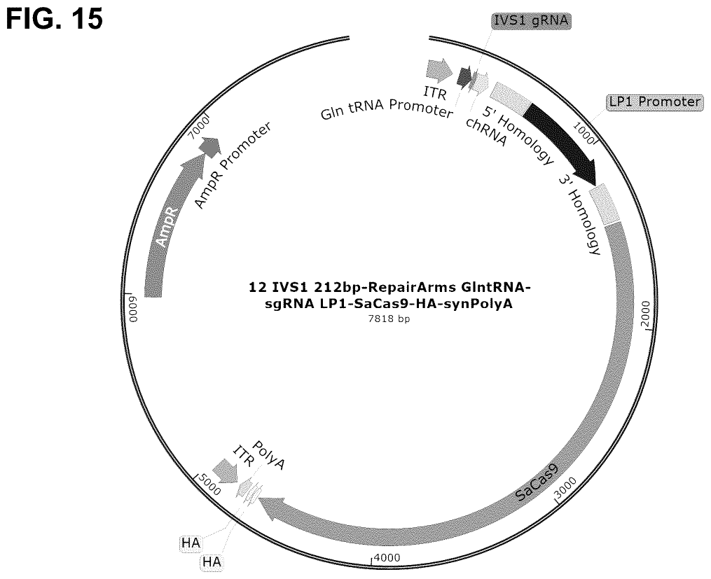

Construct 12 is titled “IVS1 212 bp-RepairArms GlntRNA-sgRNA LP1-SaCas9-HA-synPolyA”. This construct contained 212 bp repair arms flanking the LP1 promoter, which allow insertion of the LP1 promoter in intron 1. The LP1 promoter also expressed SaCas9. the LP1 promoter provided strong transgene expression and editing in the liver. Then, high levels of GAA expression in the liver served a depot and was able to send GAA to other tissues. (FIG. 15)

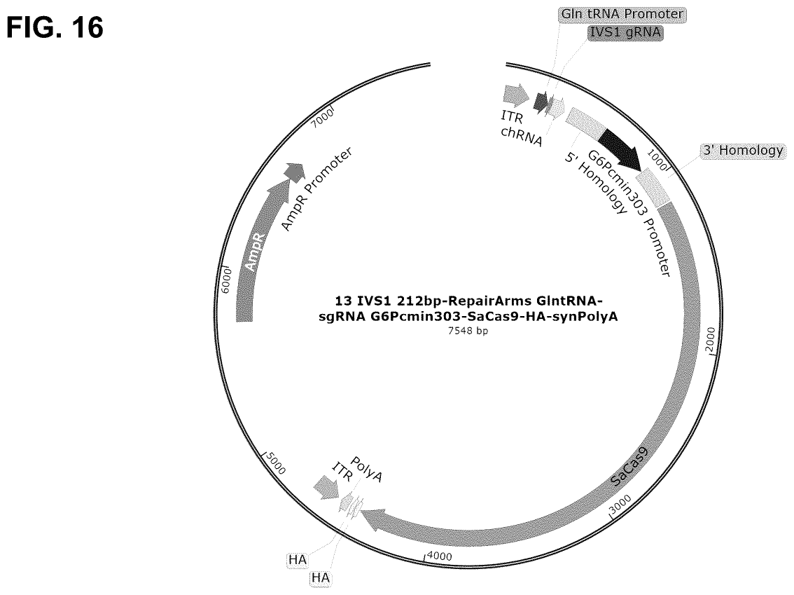

Construct 13 is titled IVS1 212 bp-RepairArms GlntRNA-sgRNA G6Pcmin303-SaCas9-HA-synPolyA”. In this construct, the G6PC promoter was used due to its strong expression in skeletal and cardiac muscle. This construct contained 212 bp repair arms flanking the G6Pcmin303 promoter, which allow insertion of the G6Pcmin303 promoter in intron 1. The G6Pcmin303 promoter also expressed SaCas9. (FIG. 16)

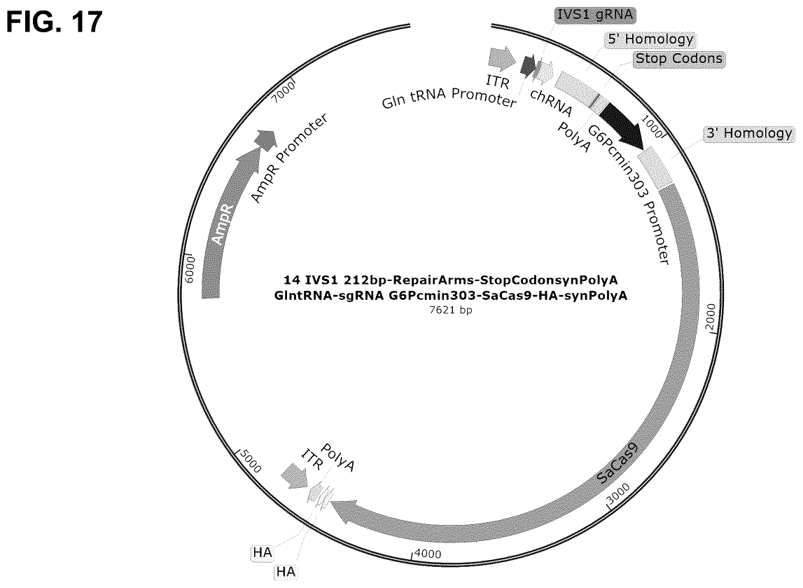

Construct 14 is titled “IVS1 212 bp-RepairArms-StopCodonsynPolyA GlntRNA-sgRNA G6Pcmin303-SaCas9-HA-synPolyA”. This construct contained 212 bp repair arms flanking the G6Pcmin303 promoter allowed insertion of the G6Pcmin303 promoter in intron 1. A polyadenylation sequence and stop codon are inserted upstream of the G6Pcmin303 to prevent expression of mutant GAA from the endogenous GAA promoter. The G6Pcmin303 promoter also expressed SaCas9. (FIG. 17)

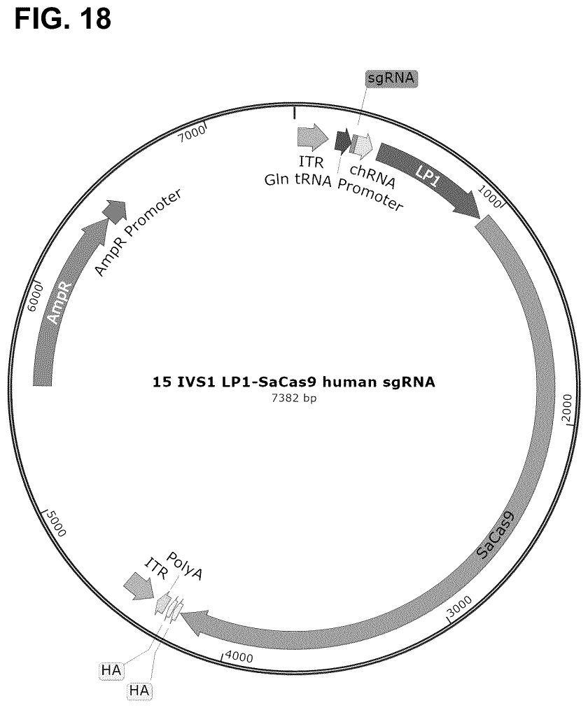

Construct 15 is titled “IVS1 LP1-SaCas9 human sgRNA”. This construct was made to demonstrate the feasibility of the liver depot gene editing approach in vivo. This construct contained the LP1 promoter to drive SaCas9 expression and a sgRNA that directed cleavage in intron 1 of the human GAA gene, which achieved integration of a GAA transgene when used with Construct 16. The in vivo editing occurs mostly in the liver where the transgenic GAA is expressed. (FIG. 18)

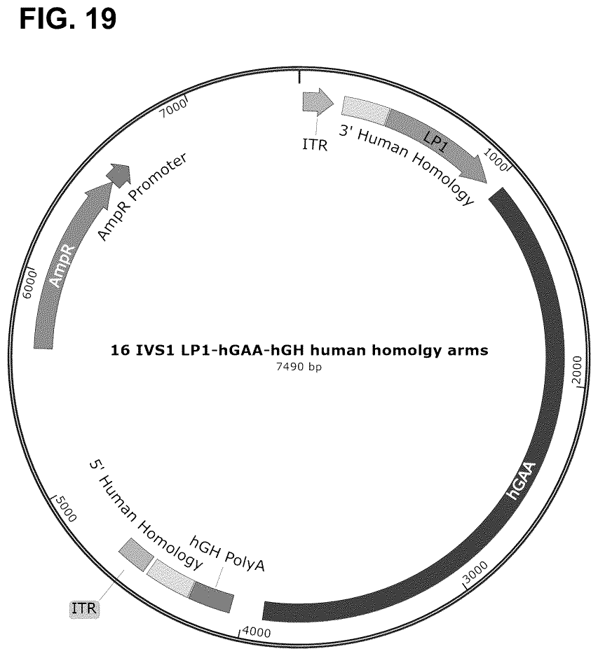

Construct 16 is titled “IVS1 LP1-hGAA-hGH human homolgy arms”. This construct contained a GAA transgene including the LP1 promoter, human GAA cDNA, and a PolyA sequence to drive human GAA expression. The transgene was flanked by short GAA repair arms from intron 1 that direct integration of the transgene into intron 1 of the GAA gene, when used with Construct 15. (FIG. 19)

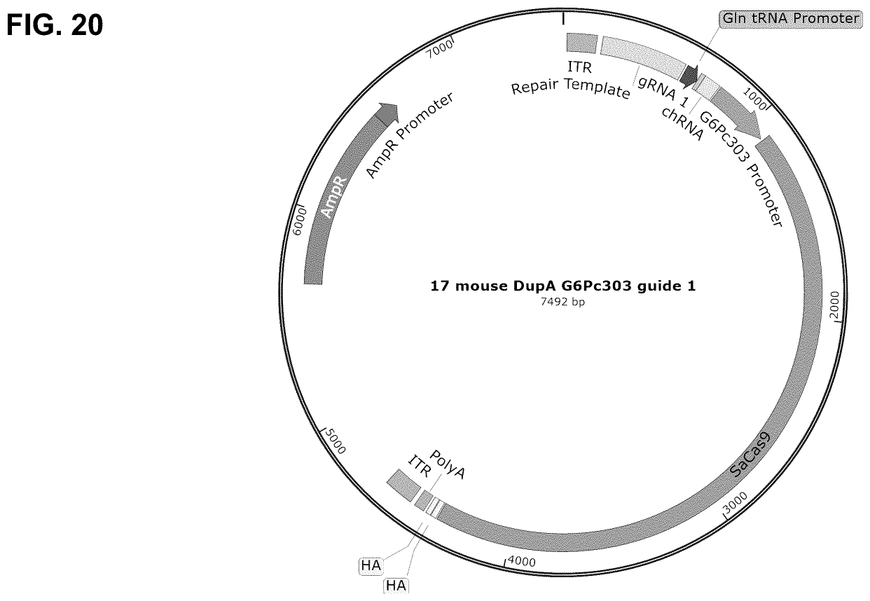

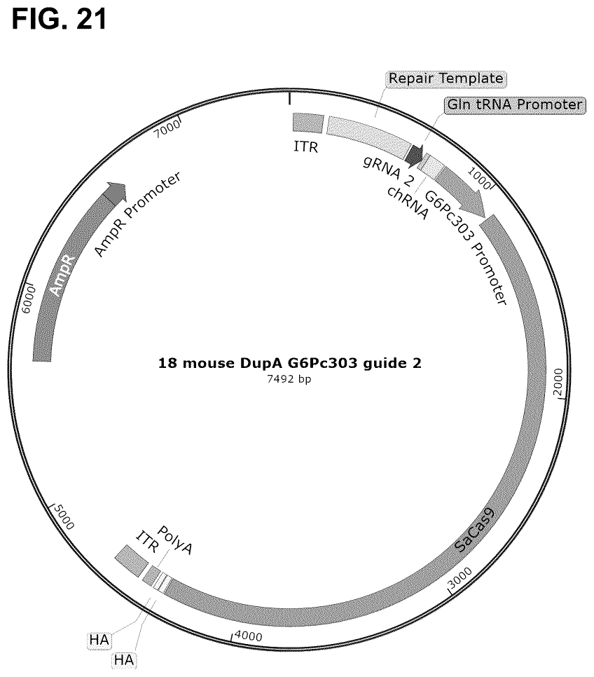

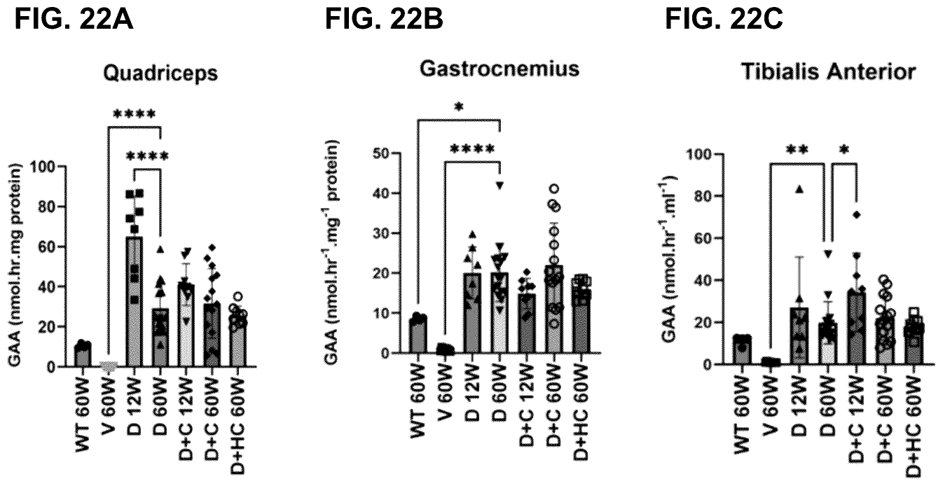

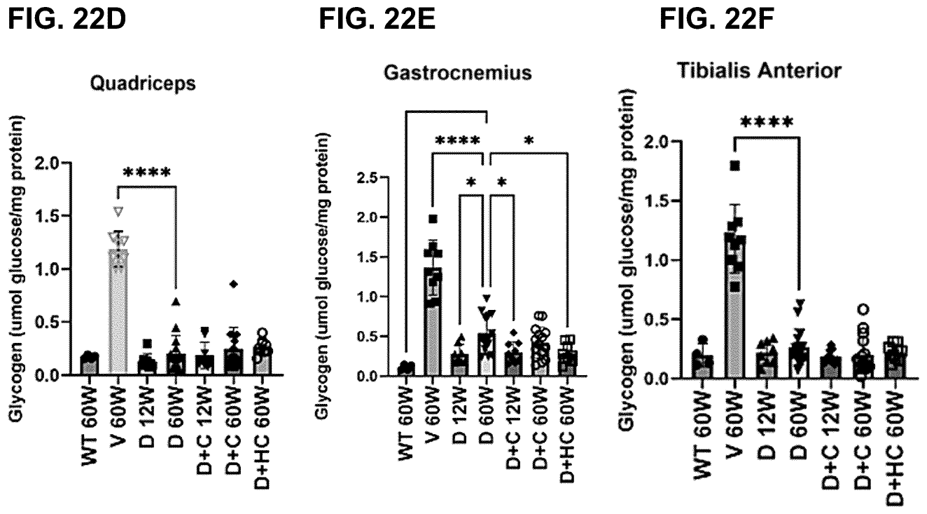

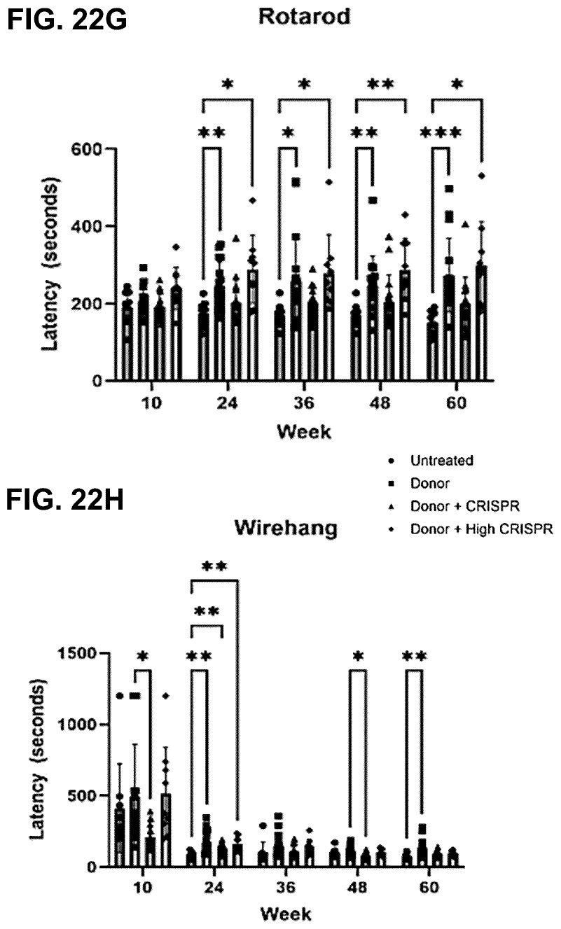

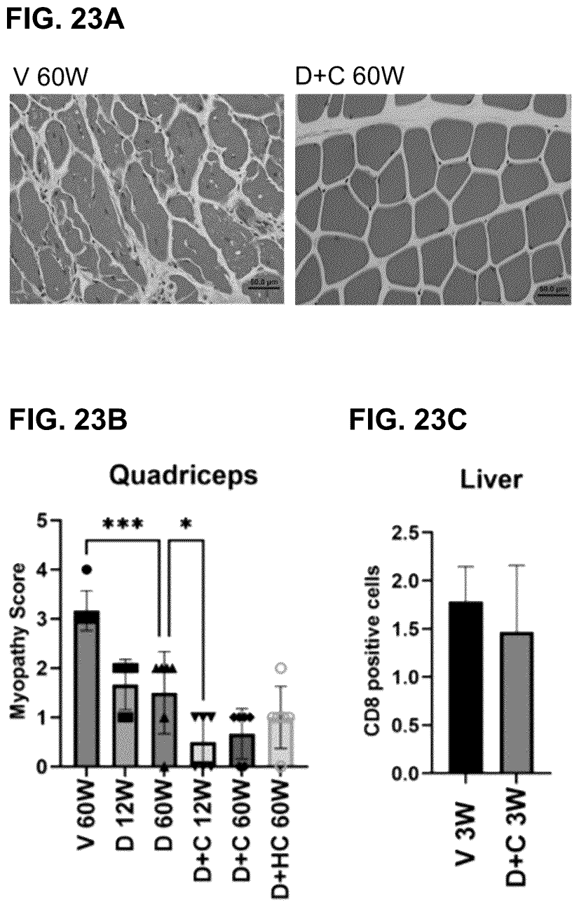

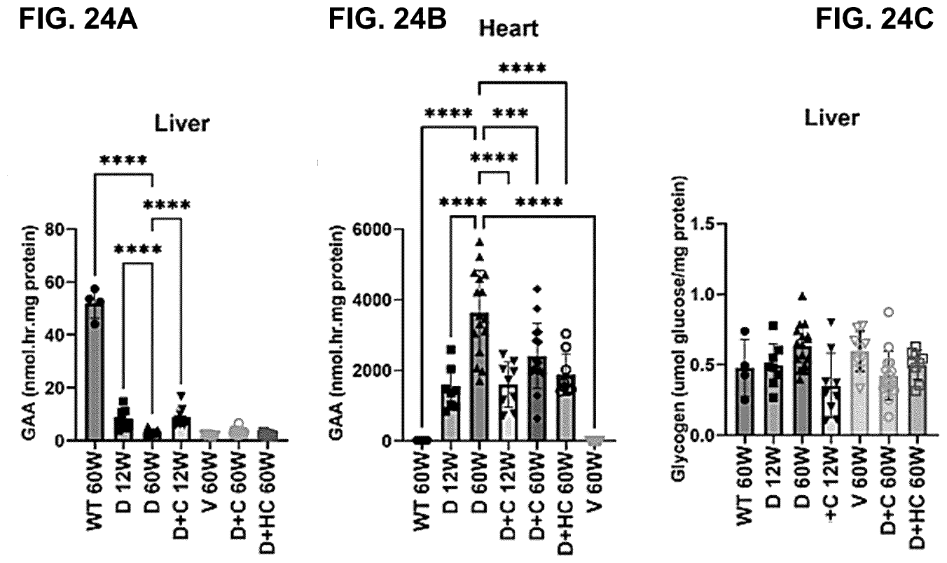

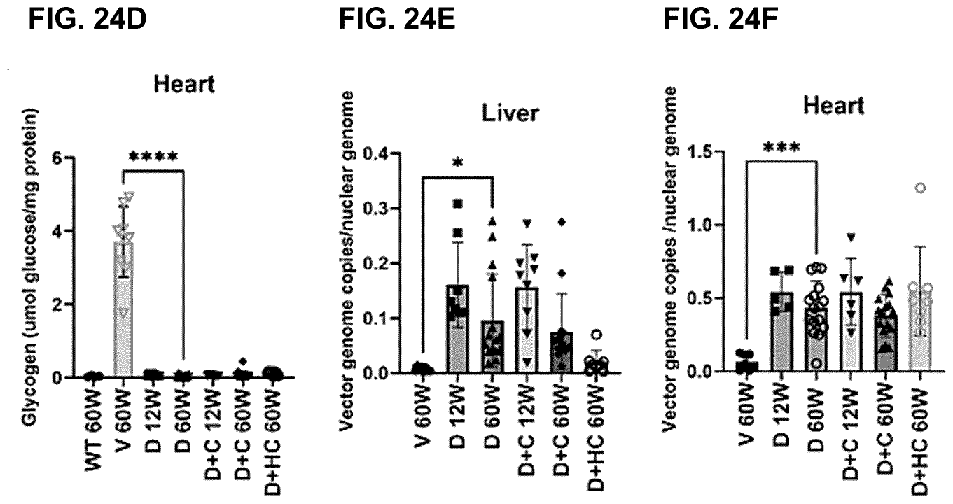

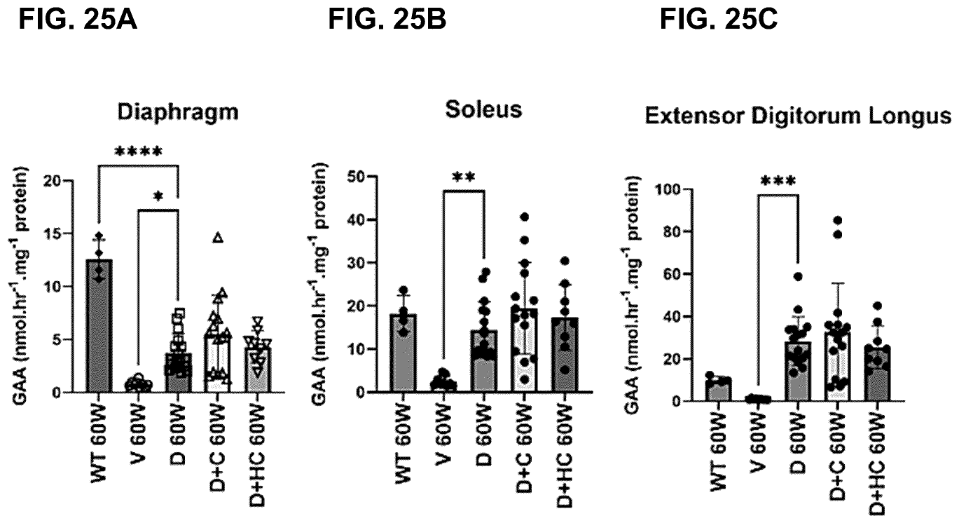

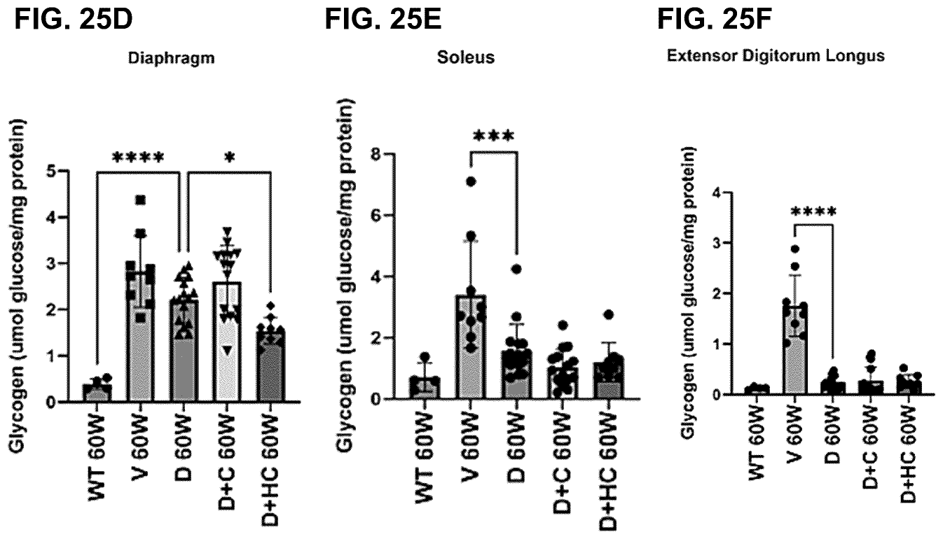

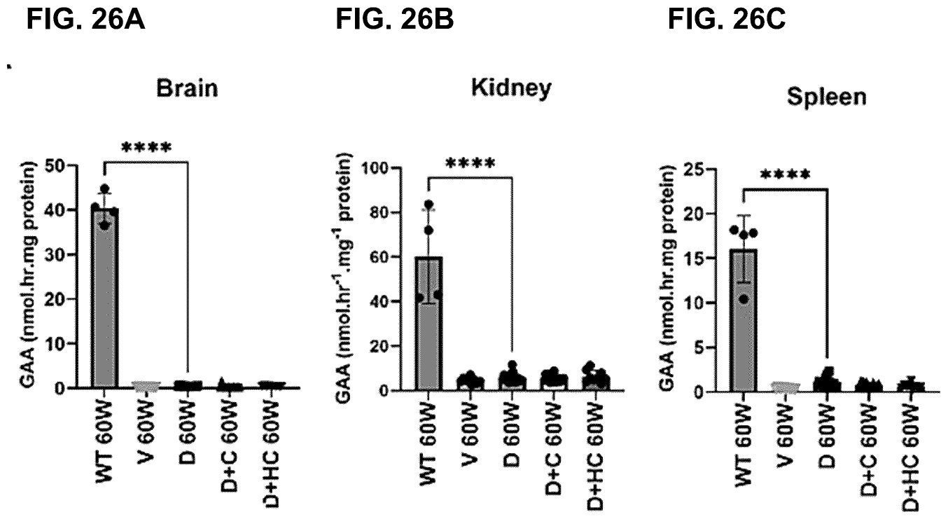

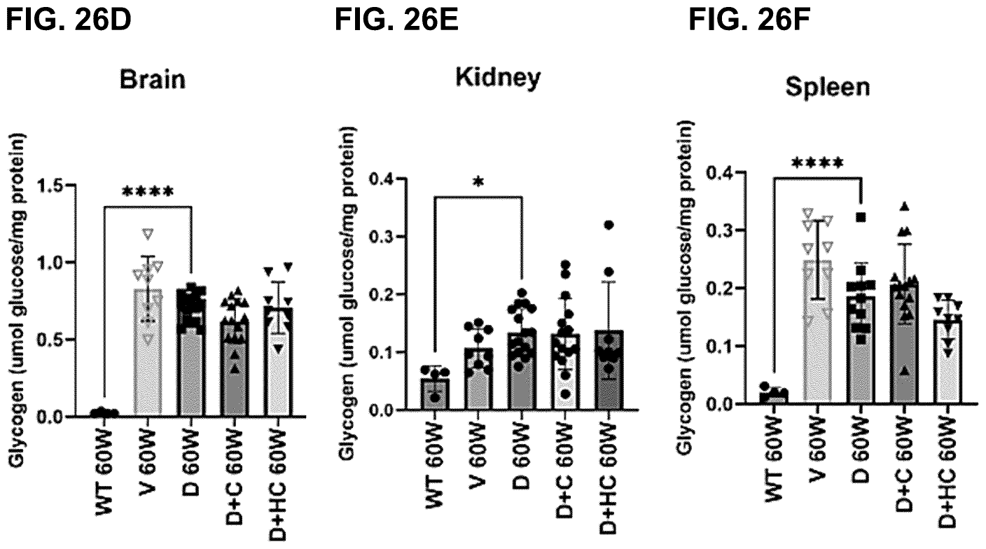

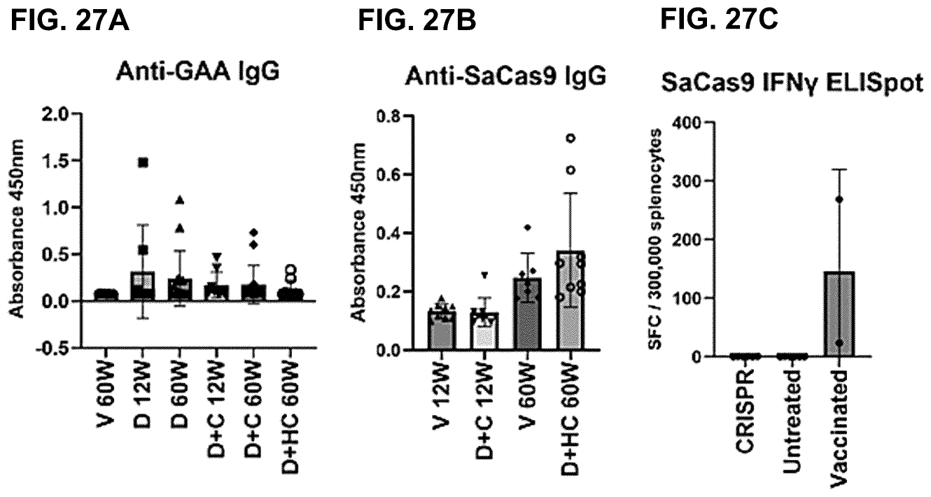

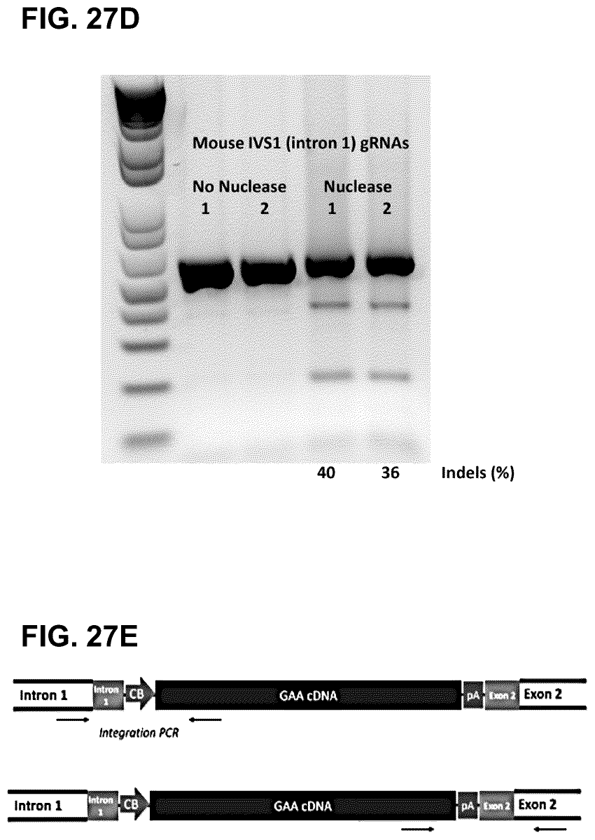

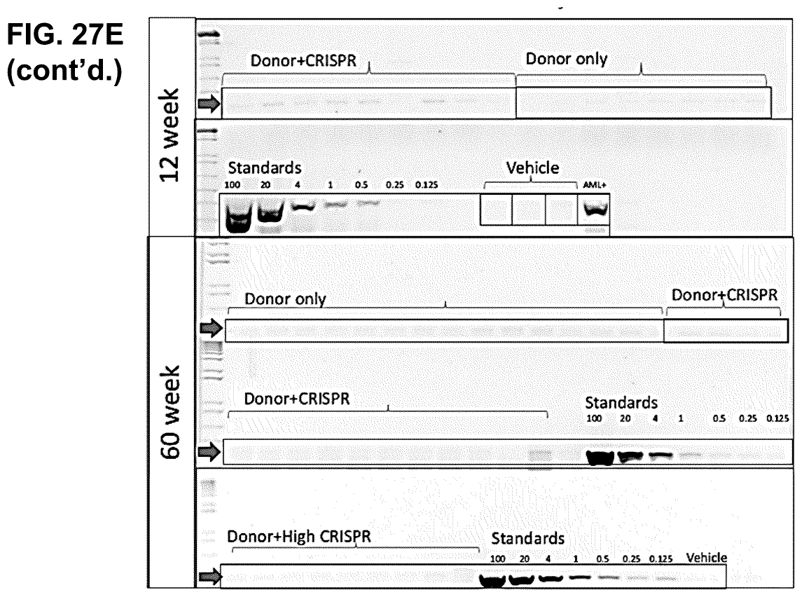

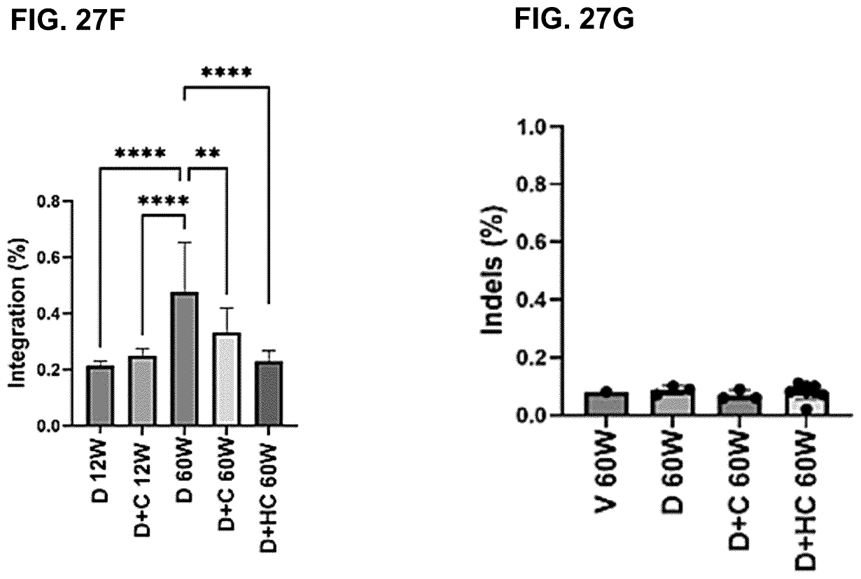

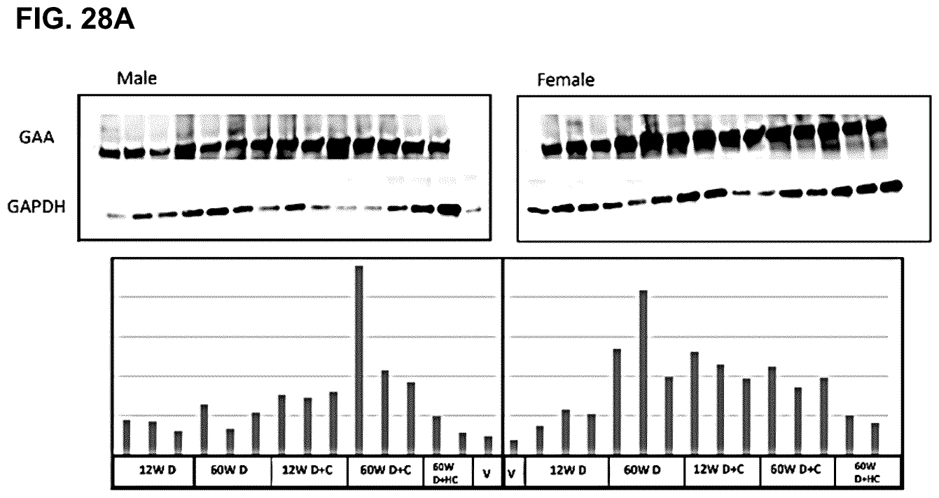

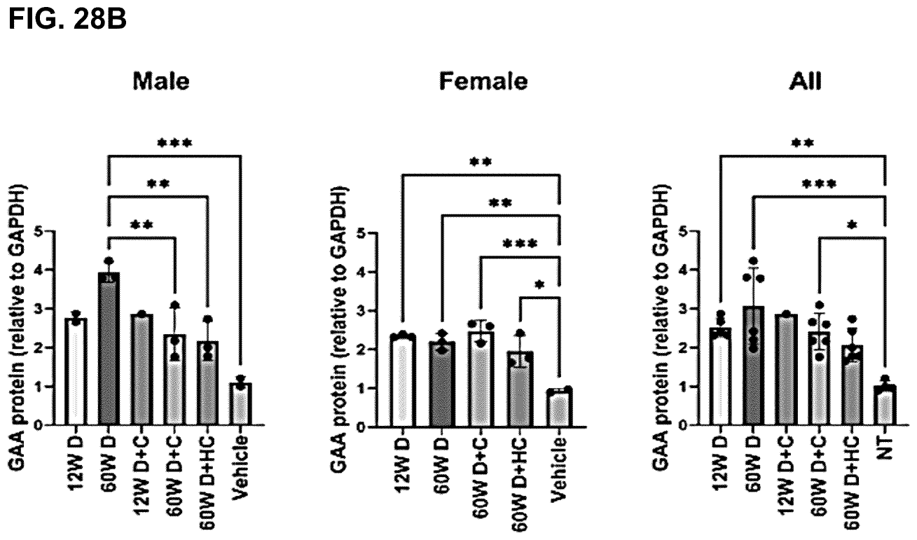

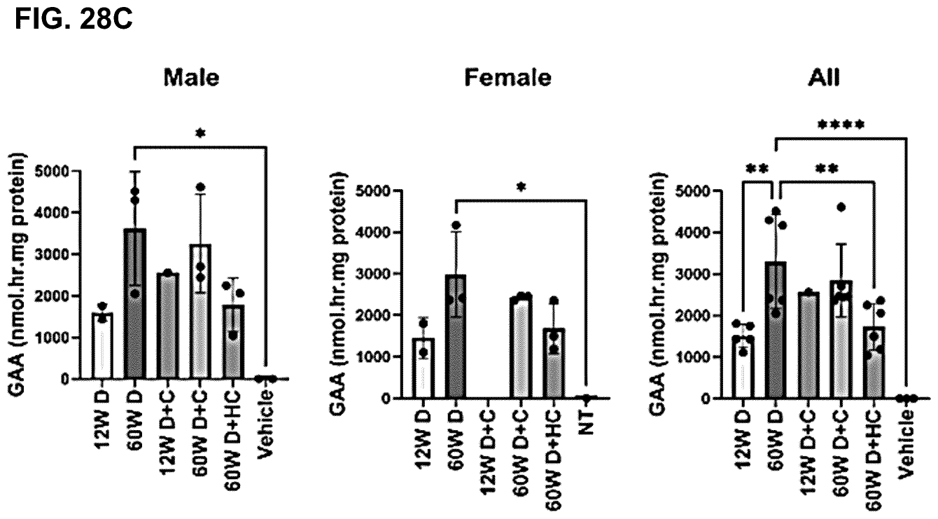

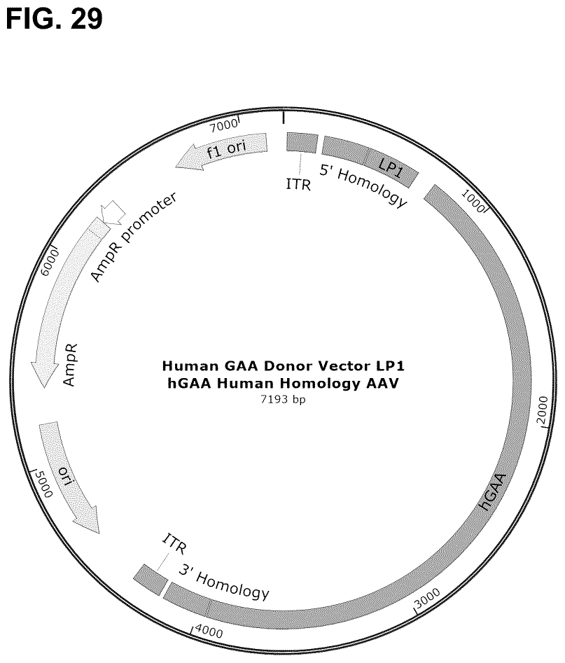

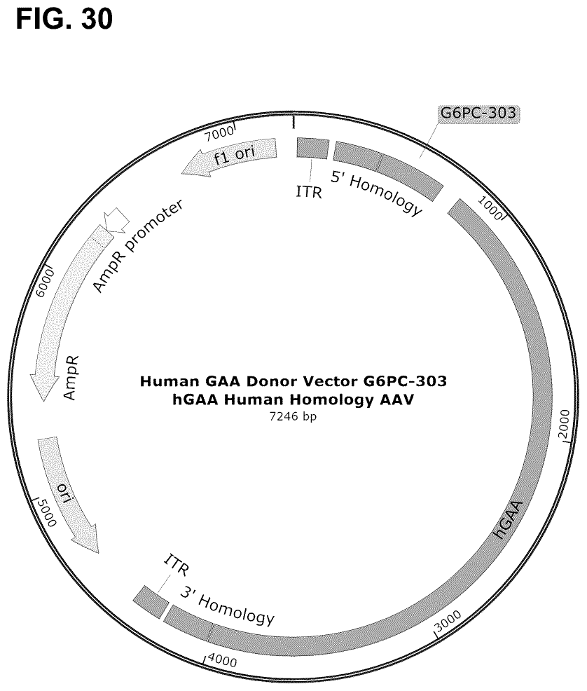

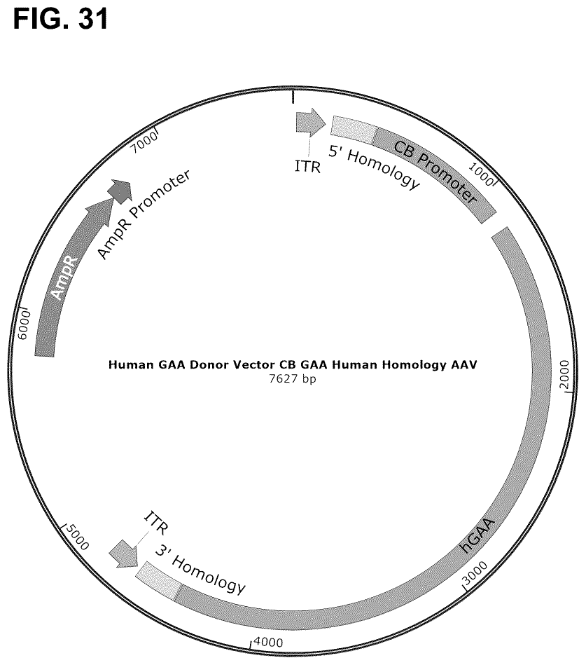

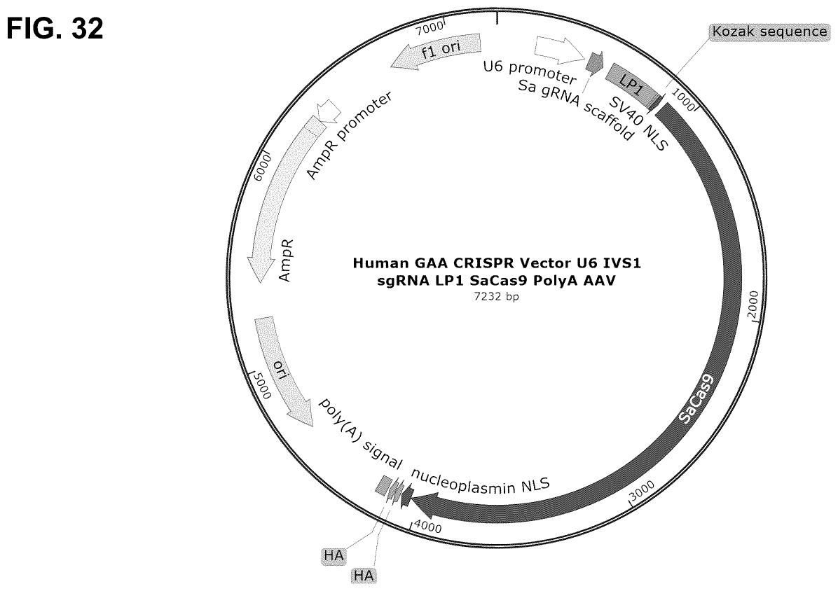

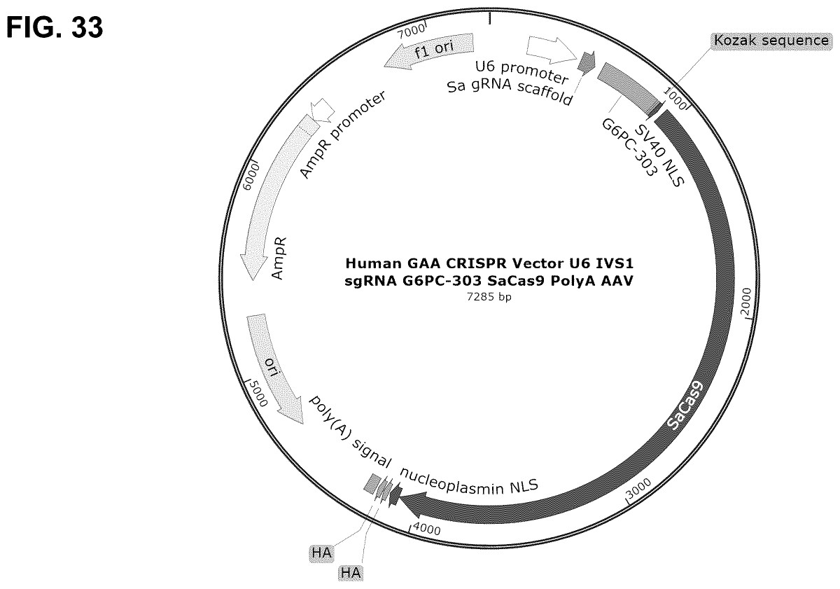

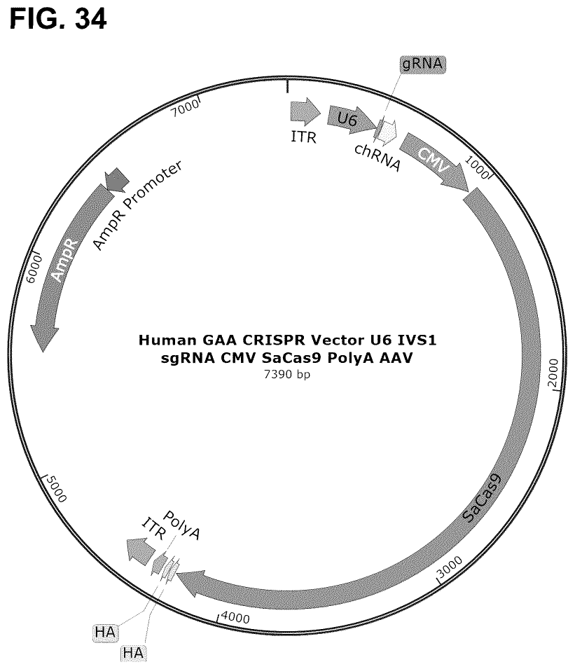

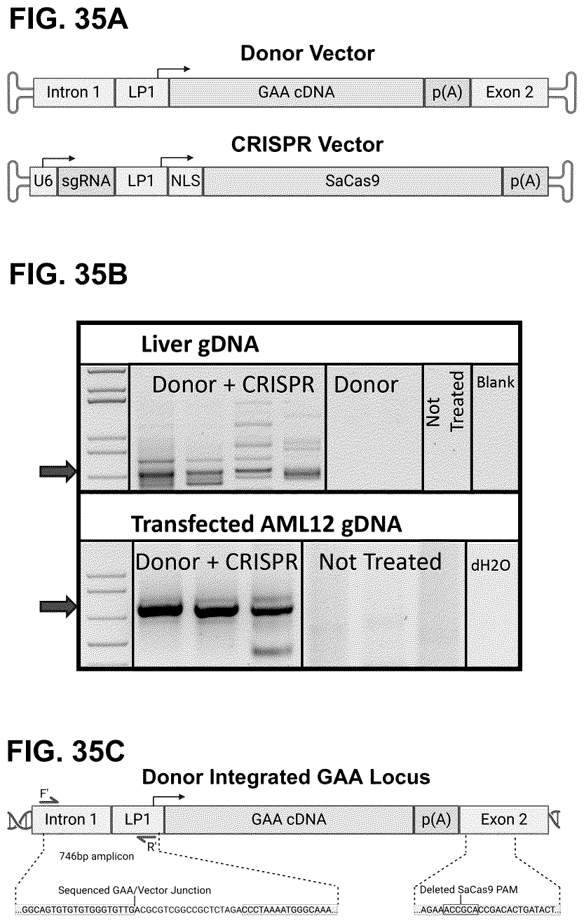

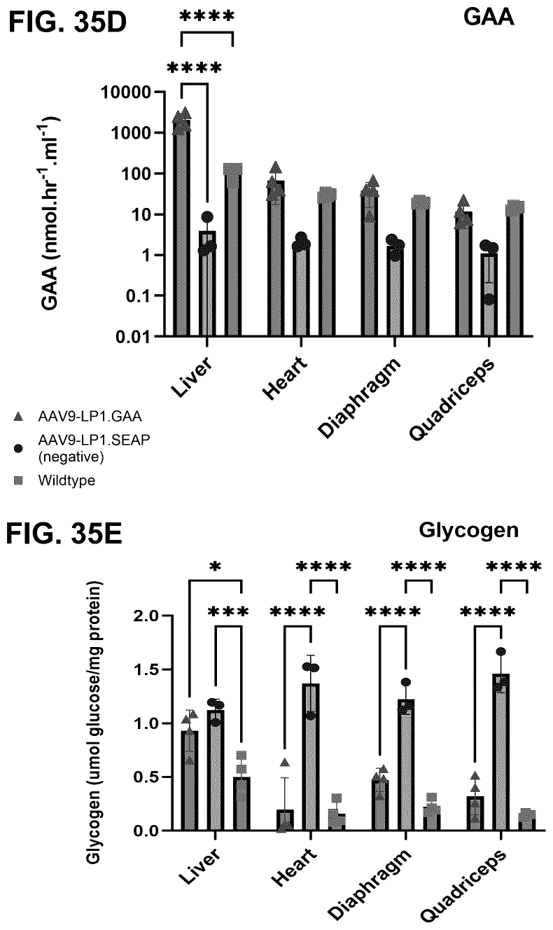

Construct 17 is titled “mouse DupA G6Pc303 guide 1”. Here, the G6PC promoter was used to generate stronger expression in skeletal and cardiac muscle. This construct contained a repair template consisting of mouse GAA sequence flanking the dupA1826 mutation, a sgRNA to cleave near the dupA1826 mutation, and a G6PC303 min promoter to drive SaCas9 expression. The substitution of the human GAA sequence would be correct the dupA1826 mutation in Pompe disease. (FIG. 20)

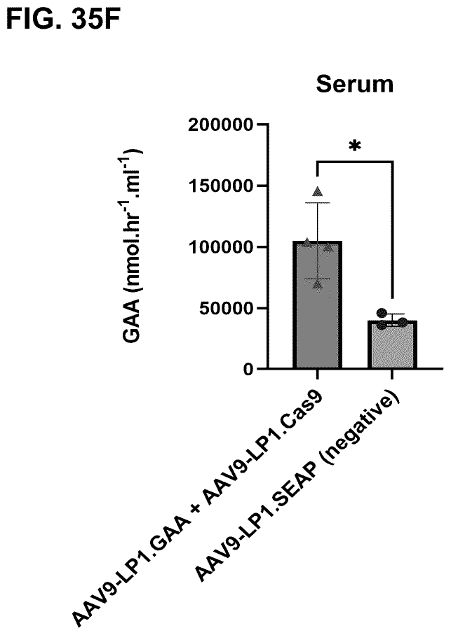

Construct 18 is titled “mouse DupA G6Pc303 guide 2”. This construct contained a repair template consisting of mouse GAA sequence flanking the dupA1826 mutation, a sgRNA to cleave near the dupA1826 mutation (different from the one in Construct 17), and a G6PC303 min promoter to drive SaCas9 expression. The substitution of the human GAA sequence would be correct the dupA1826 mutation in Pompe disease. (FIG. 21)

TABLE 1
Summary of Sequences

| ID# | Description |
|---|---|
| 1 | delT525 623 bp-Repair U6-sgRNA CMV-SaCas9-HA-synPolyA |
| 2 | delt525 400 bp-Repair GlntRNA-sgRNA CB-SaCas9-HA-synPolyA |
| 3 | delT525 400 bp-Repair GlntRNA-sgRNA G6Pcmin303-SaCas9-HA-synPolyA |
| 4 | IVS1 212 bp-RepairArms GlntRNA-sgRNA CMV-SaCas9-HA-synPolyA |
| 5 | IVS1 210 bp-RepairArms GlntRNA-sgRNA EF1a-SaCas9-HA-synPolyA |
| 6 | m1826DupA 404 bp-Repair GlntRNA-sgRNA1 CMV-SaCas9-HA-synPolyA |
| 7 | m1826DupA 404 bp-Repair GlntRNA-sgRNA2 CMV-SaCas9-HA-synPolyA |
| 8 | mIVS1 GlntRNA-sgRNA LP1-SaCas9-HA-synPolyA |
| 9 | mIVS1 212 bp-RepairArms LP1-hGAA-hGHPolyA |
| 10 | delT525 623 bp-Repair GlntRNA-sgRNA EF1a-SaCas9-HA-synPolyA |
| 11 | IVS1 212 bp-RepairArms GlntRNA-sgRNA CB-SaCas9-HA-synPolyA |
| 12 | IVS1 212 bp-RepairArms GlntRNA-sgRNA LP1-SaCas9-HA-synPolyA |
| 13 | IVS1 212 bp-RepairArms GlntRNA-sgRNA G6Pcmin303-SaCas9-HA-synPolyA |
| 14 | IVS1 212 bp-RepairArms-StopCodonsynPolyA GlntRNA-sgRNA G6Pcmin303- |
| | SaCas9-HA-synPolyA |
| 15 | IVS1 LP1-SaCas9 human sgRNA |
| 16 | IVS1 LP1-hGAA-hGH human homolgy arms |
| 17 | mouse DupA G6Pc303 guide 1 |
| 18 | mouse DupA G6Pc303 guide 2 |
| 19 | Cas9 |
| 20 | Inverted Terminal Repeat |
| 21 | Inverted Terminal Repeat |
| 22 | Inverted Terminal Repeat |
| 23 | HA Tag |
| 24 | HA Tag |
| 25 | chRNA |
| 26 | GAA Repair Template |
| 27 | GAA Repair Template |
| 28 | GAA Repair Template |
| 29 | Human delT525 gRNA |
| 30 | IVS1 gRNA |
| 31 | m1826DupA gRNA1 |
| 32 | m1826DupA gRNA1 + plus PAM |
| 33 | m1826DupA gRNA2 |
| 34 | m1826DupA gRNA2 + PAM |
| 35 | mIVS1 gRNA |
| 36 | mIVS1 gRNA + PAM |
| 37 | scrambled sgRNA |
| 38 | PAM1 |
| 39 | PAM2 |
| 40 | U6 Promoter |
| 41 | CMV-min Promoter |
| 42 | CMV Promoter |
| 43 | CMV Promoter |
| 44 | CB Promoter |
| 45 | G6PC min303 Promoter |
| 46 | EF1α Promoter |
| 47 | Liver Promoter |
| 48 | Gln tRNA Promoter |
| 49 | Inserted BamHI Site |
| 50 | SV40 NLS |
| 51 | Nuclear Localization Signal |
| 52 | PolyA |
| 53 | PolyA |
| 54 | 5′ Homology Arm |
| 55 | 5′ Homology Arm |
| 56 | 5′ Homology Arm |
| 57 | 5′ Mouse Homology Arm |
| 58 | 3′ Mouse Homology Arm |
| 59 | 3′ Homology Arm |
| 60 | 3′ Homology Arm |
| 61 | 3′ Homology Arm |
| 62 | hGAA |

### Summary of Examples

The ability to correct individual mutations with a single AAV vector will repair a patient's GAA gene to correct GAA deficiency in corrected cells and in the daughter cell progeny from these corrected cells. The repaired GAA gene will have normal function and will treat Pompe disease. The single vector strategy is more efficient than other strategies for genome editing that require two AAV vectors to transduce each cell to achieve gene correction.

The underlying strategy depends upon the stable transduction of hepatocytes through genome editing, which prevents the loss of episomal AAV genomes due to cell division that limits the efficacy of gene replacement therapy. Increasingly genome editing studies use CRISPR/Cas9 as a nuclease, due to its flexibility and high nuclease activity. Nuclease-free strategies have been developed for genome editing in hemophilia B (Barzel A, et al. (2015) Nature. 517:360-364); however, the HDR efficiency was less than 1% and too low to treat inherited metabolic diseases like Pompe disease. Thus, that the disclosed CRISPR/Cas9-mediated genome editing composition and methods can create a liver depot for the treatment of Pompe disease will enhance the treatment of very young patients.

The disclosed strategy was developed specifically to integrate the transgene at the site for the c.-32-13T->G variant that is present in ˜90% of patients with late-onset Pompe disease and comprises 45% of all variants (i.e., the “IVS1” variant (Kroos M A, et al. (2007) Neurology. 68:110-115; Reuser A J, et al. (2019b) Hum Mutat. 40:2146-2164a)). However, the two-vector strategy disclosed herein to insert a functional GAA transgene within a mutant GAA gene can be use to treat any Pompe patient, regardless of pathogenic variant or variants causing Pompe disease. (FIG. 3A-FIG. 3B). The disclosed strategy for genome editing addresses the limitations of gene therapy by stably integrating the transgene in chromosomal DNA. Genome editing has been initiated to correct a mutation or integrate a transgene as a method to stably treat liver metabolic diseases and hemophilia, including GSD Ia, hemophilia B, ornithine transcarbamylase deficiency, and phenylketonuria (Landau D J, et al. (2016) Mol Ther. 24:697-706; Yang Y, et al. (2016) Nat Biotechnol. 34:334-338; Ohmori T, et al. (2017) Sci Rep. 7:4159; Richards D Y, et al. (2020) Mol Ther Meth Clin Dev. 17:234-245).

The only type of mutation not corrected by the single vector method would be a large GAA gene deletion; however, large deletions of an entire exon comprise a small fraction of pathogenic variants on Pompe disease. Furthermore, patients are typically compound heterozygotes and would have a repairable variant paired with any large deletion, in which case the patient with a large deletion could be treated by repairing the other pathogenic variants in their cells. The rationale is that being a carrier for Pompe disease causes no symptoms. In a large study of 1,079 patients with Pompe disease, only one patient was homozygous for a large deletion and would not be treatable by a single vector genome editing strategy (Reuser A J, et al. (2019b) Hum Mutat. 40:2146-2164).

These experiments demonstrated that genome editing can be used to treat Pompe disease, either by correcting individual mutations or by stably producing GAA regardless of mutation. Either method addressed the limitation of episomal AAV vector genomes being lost from cells during cell division. Stable treatment of Pompe disease with genome editing will provide treatment for young patients, addressing the need for treatment of Pompe disease early in life. A single dose of an AAV vector or AAV vectors to accomplish genome editing will replace ERT with its limitations (e.g., incomplete treatment, need for frequent treatment, immune responses prevent benefits) and overcomes the need for repeated administration of AAV vectors for standard gene replacement therapy.

### KEY TAKEAWAYS

The compositions and methods disclosed herein provided several key takeaways.

Importantly, all the disclosed gene editing components (including the repair template) can be successfully packaged into a single AAV. By designing a single AAV vector to efficiently deliver all the components needed to achieve genome editing, the repair template used here—which was approximately 200 bp homology arms on each side of the mutation to be corrected—was smaller than any previous sequence used in previous genome editing experiments. Small repair templates and small promoters enabled the single AAV vector design. By correcting the common IVS1 and 525deltaT mutations, this method can stably treat most Pompe patients with genome editing. This makes delivery of the vector more efficient and improves the efficacy of the gene editing.

Moreover, the insertion of a small promoter in intron 1 to drive normal GAA expression is groundbreaking. The GAA gene has a start codon in exon 2, which allows the insertion of a promoter in intron 1 to drive full-length GAA expression to treat Pompe disease. Simultaneously, the disclosed methods corrected the most common mutation in Pompe disease, known at the IVS1 mutation, by inserting wildtype GAA sequence to replace the IVS1 mutation. Inserting a strong promoter in intron 1 drives expression from the liver to secrete GAA into the blood accompanied by mannose-6-phosphate receptor mediated uptake into the heart and skeletal muscle to treat Pompe disease. Insertion of a strong, liver-specific promoter in intron 1 overexpressed GAA and converted the liver into a depot for GAA production. This approach represents a unique and potentially curative approach to GAA gene editing and addresses the main issue for GAA gene therapy—the loss of AAV vectors from transduced tissues over time. This was only possible because the approximately 200 bp homology arms in the repair template fit into an AAV vector with the GAA transgene (as longer homology arms would exceed AAV's packaging capacity).

Accordingly, the disclosed compositions and methods demonstrated that a transgene (having smaller repair/homology arms) can be incorporated into the host genome, which affords the skilled clinician the ability to treat any mutation in the GAA gene that gives rise to Pompe disease. The genome editing achieved by the disclosed compositions and methods is permanent and the resulting expression of GAA is sustained.

Finally, while the disclosed compositions and methods successfully targeted multiple and different mutations in the GAA locus (e.g., Pompe disease), the disclosed compositions and methods can be tailored to successfully target other genetic diseases and disorders (see, for example, Section VII (F) titled “Methods of Treating and/or Preventing a Genetic Disease or Disorder” provided supra).

These accomplishments represent surprising and unexpected contributions to the Pompe community (as well as the community focused on treating and/or preventing and/or curing other genetic diseases and defects) and exceeded the reasonable expectations of the skilled person.

What is claimed is:

1. A nucleic acid molecule, comprising:
a nucleic acid sequence encoding a repair template, wherein the repair template encodes acid alpha-glucosidase (GAA) or a fragment thereof;
a promoter operably linked to the repair template;
a first nucleic acid sequence having homology to a sequence in intron 1 of GAA, wherein the first homologous sequence is 5′ of the repair template; and
a second nucleic acid sequence having homology to a sequence in exon 2 of GAA, wherein the second homologous sequence is 3′ of the repair template.

2. The nucleic acid molecule of claim 1, wherein the promoter operably linked to the repair template comprises a sequence having at least 90% identity to the sequence set forth in any one of SEQ ID NO:44-SEQ ID NO:58.

3. The nucleic acid molecule of claim 1, wherein the repair template encoding GAA or a fragment thereof comprises a sequence having at least 90% identity to the sequence set forth in SEQ ID NO: 73, SEQ ID NO:74, SEQ ID NO:75, SEQ ID NO:76, or SEQ ID NO:100.

4. The nucleic acid molecule of claim 1,
wherein the first homologous sequence comprises about 175 base pairs (bp) to about 225 bp, and
wherein the second homologous sequence comprises about 175 bp to about 225 bp.

5. The nucleic acid molecule of claim 4,
wherein the first homologous sequence comprises the sequence set forth in any one of SEQ ID NO:54-SEQ ID NO:56, and
wherein the second homologous sequence comprises the sequence set forth in any one of SEQ ID NO:59-SEQ ID NO:61.

6. The nucleic acid molecule of claim 1, further comprising one or more inverted terminal repeats (ITRs), one or more poly A sequences, or any combination thereof.

7. A nucleic acid molecule, comprising:
a promoter operably linked to a nucleic acid sequence encoding a Cas9 nuclease, and
a promoter operably linked to a nucleic acid sequence encoding a guide RNA (gRNA).

8. The nucleic acid molecule of claim 7,
wherein the gRNA targets a protospacer adjacent motif (PAM) sequence at or near the IVS1 variant near the boundary between intron 1 and exon 2 in the GAA gene; and
wherein the Cas9 creates a double-strand break within or near the IVS1 variant in the GAA locus that results in a permanent integration of a repair template, and
wherein the repair template encodes GAA or a fragment thereof.

9. The nucleic acid molecule of claim 7, wherein the promoter operably linked to the nucleic acid sequence encoding the Cas9 nuclease comprises a sequence having at least 90% identity to the sequence set forth in any one of SEQ ID NO:44-SEQ ID NO:58.

10. The nucleic acid molecule of claim 7, wherein the promoter operably linked to the nucleic acid sequence encoding the guide RNA comprises a sequence having at least 90% identity to the sequence set forth in SEQ ID NO:73, SEQ ID NO:74, SEQ ID NO:75, SEQ ID NO: 76, or SEQ ID NO:100.

11. The nucleic acid molecule of claim 7, further comprising one or more nuclear localization signals (NLS), one or more poly A sequences, one or more hemagglutinin (HA) tags, one or more CRISPR/RNA activating sequences (chRNA), or any combination thereof.

12. A recombinant adeno-associated virus (rAAV) vector, comprising: the nucleic acid molecule of claim 1.

13. A recombinant adeno-associated virus (rAAV) vector, comprising: the nucleic acid molecule of claim 7.

14. A gene editing system, comprising:
(i) a first rAAV vector comprising a nucleic acid sequence encoding a repair template, wherein the repair template encodes acid alpha-glucosidase (GAA), a promoter operably linked to the repair template, and a first nucleic acid sequence having homology to a sequence in intron 1 of GAA, wherein the first homologous sequence is 5′ of the repair template, and a second nucleic acid sequence having homology to a sequence in exon 2 of GAA wherein the second homologous sequence is 3′ of the repair template; and
(ii) a second rAAV vector comprising a promoter operably linked to a nucleic acid sequence encoding a Cas9 nuclease, and a promoter operably linked to a nucleic acid sequence encoding a guide RNA (gRNA).

15. A pharmaceutical formulation comprising the gene editing system of claim 14.

16. A method of treating a subject, the method comprising:
administering to a subject having Pompe disease a therapeutically effective amount of the pharmaceutical formulation of claim 15;
wherein, following expression of the encoded nucleic acids, the expression level and/or the activity level of GAA in one or more cells in the subject is increased.

17. The method of claim 16, further comprising administering to the subject one or more additional therapeutic agents, wherein the one or more additional therapeutic agents comprise enzyme replacement therapy, gene therapy, mRNA therapy, small molecule therapy, substrate reduction therapy, or any combination thereof.

18. The method of claim 16, further comprising administering to the subject one or more immune modulators, wherein the one or more immune modulators comprise methotrexate, rituximab, intravenous gamma globulin, or bortezomib, or any combination thereof.

19. The method of claim 16, further comprising administering to the subject one or more proteasome inhibitors, wherein the one or more proteasome inhibitors comprise bortezomib, carfilzomib, marizomib, ixazomib, oprozomib, or any combination thereof.

20. The method of claim 16, further comprising restoring one or more aspects of cellular homeostasis and/or cellular functionality and/or metabolic dysregulation.


SEQUENCE LISTING (109 sequences)

SEQ ID NO: 1 - DNA, 8030 bp, synthetic construct
  source 1..8030: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctgaattcctagacagcagggcaacacccaccctggccaccttaccccacctgcctgggtgctgcagtgccagccgcggttgatgtctcagagctgctttgagagccccgtgagtgccgcccctcccgcctccctgctgagcccgctttcttctcccgcaggcctgtaggagctgtccaggccatctccaaccatgggagtgaggcacccgccctgctcccaccggctcctggccgtctgcgccctcgtgtccttggcaaccgctgcactcctggggcacatcctactacatgatttcctgctggttccccgagagctgagtggatcctccccagtcctggaggagacgcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatggcatgggtaccgaagtattatgcacgacatctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggctccctgctggtgagctgggttttagtactctggaaacagaatctactaaaacaaggcaaaatgccgtgtttatctcgtcaacttgttggcgagattttttctcgaggtcggaggatccacaaaggctagccggtctatgcatgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagctcgtttagtgaaccgtcagatcgcctggagacaccatccacgctgttttgacctccatagaagagaccgggaccgatccagcctaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 2 - DNA, 7670 bp, synthetic construct
  source 1..7670: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccaatttcgagctcggtaccccgcaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggatcctccccagtcctggaggagacgcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccatctagacatgggttccatggtgtaatggttagcactctggactctgaatccagcgatccgagttcaaatctcggtggaacctggctccctgctggtgagctgggttttagtactctggaaacagaatctactaaaacaaggcaaaatgccgtgtttatctcgtcaacttgttggcgagattttttctcgagccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 3 - DNA, 7488 bp, synthetic construct
  source 1..7488: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccaatttcgagctcggtaccccgcaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggatcctccccagtcctggaggagacgcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccatctagacatgggttccatggtgtaatggttagcactctggactctgaatccagcgatccgagttcaaatctcggtggaacctggctccctgctggtgagctgggttttagtactctggaaacagaatctactaaaacaaggcaaaatgccgtgtttatctcgtcaacttgttggcgagattttttctcgagctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 4 - DNA, 7643 bp, synthetic construct
  source 1..7643: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagacatgggttccatggtgtaatggttagcactctggactctgaatccagcgatccgagttcaaatctcggtggaacctcagagctgctttgagagccccgttttagtactctggaaacagaatctactaaaacaaggcaaaatgccgtgtttatctcgtcaacttgttggcgagattttttctcgaggtcggaggatccacaaaggctagcttcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcggacgcgttgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagctcgtttagtgaaccgtcagatcgcctggagacaccatccacgctgttttgacctccatagaagagaccgggaccgatccagcctaactagtttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactccaaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 5 - DNA, 7645 bp, synthetic construct
  source 1..7645: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacccctggggacattctaagcgtgtttgatttgtaacattttagcagactgtgcaagtgctctgcactcccctgctggagcttttctcgcccttccttctggccctctccccagtctagacagcagggcaacacccaccctggccaccttaccccacctgcctgggtgctgcagtgccagccgcggttgatgtctcagagctgctttgagagcgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagcccgtcagtgccgcccctcccgcctccctgctgagcccgctttcttctcccgcaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagtctagacatgggttccatggtgtaatggttagcactctggactctgaatccagcgatccgagttcaaatctcggtggaacctcagagctgctttgagagccccgttttagtactctggaaacagaatctactaaaacaaggcaaaatgccgtgtttatctcgtcaacttgttggcgagattttttctcgaggtcggaggatccacaaaggctagccagtgggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 6 - DNA, 7597 bp, synthetic construct
  source 1..7597: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccaatttcgagctcggtaccgcctgactgtacctagcatactttaaagatggcctctctttcccacccattgtgatctactaaactatcagttcctcttggccctgatccccttgctgctggcccccatgctctgttccagcacacgggtgtgtatgacccaggtgagcccaggtctctgtgttctcatcccactcagggccctggtcaagacgcggggaacacgaccgtttgtgatctcccgctcaaccttctcgggccacggaagatacgctggtcactggactggggatgtgcggagctcttgggagcatcttgcatactctgtgccaggtgagtacaagctgcctgcagggctctggggtcttttctgggggtctttgggctgtgggctcaccactgcagatggtattgtgatagcttggtctagacatgggttccatggtgtaatggttagcactctggactctgaatccagcgatccgagttcaaatctcggtggaacctgatcacaaagggtcgtgttccgttttagtactctggaaacagaatctactaaaacaaggcaaaatgccgtgtttatctcgtcaacttgttggcgagattttttctcgagacggtctatgcatgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagctcgtttagtgaaccgtcagatcgcctggagacaccatccacgctgttttgacctccatagaagagaccgggaccgatccagcctagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 7 - DNA, 7597 bp, synthetic construct
  source 1..7597: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccaatttcgagctcggtaccgcctgactgtacctagcatactttaaagatggcctctctttcccacccattgtgatctactaaactatcagttcctcttggccctgatccccttgctgctggcccccatgctctgttccagcacacgggtgtgtatgacccaggtgagcccaggtctctgtgttctcatcccactcagggccctggtcaagacgcggggaacacgaccgtttgtgatctcccgctcaaccttctcgggccacggaagatacgctggtcactggactggggatgtgcggagctcttgggagcatcttgcatactctgtgccaggtgagtacaagctgcctgcagggctctggggtcttttctgggggtctttgggctgtgggctcaccactgcagatggtattgtgatagcttggtctagacatgggttccatggtgtaatggttagcactctggactctgaatccagcgatccgagttcaaatctcggtggaacctaaggttgagcgggagatcacagttttagtactctggaaacagaatctactaaaacaaggcaaaatgccgtgtttatctcgtcaacttgttggcgagattttttctcgagacggtctatgcatgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagctcgtttagtgaaccgtcagatcgcctggagacaccatccacgctgttttgacctccatagaagagaccgggaccgatccagcctagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 8 - DNA, 7382 bp, synthetic construct
  source 1..7382: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagacatgggttccatggtgtaatggttagcactctggactctgaatccagcgatccgagttcaaatctcggtggaacctttccacctcctggcagtgtgtgttttagtactctggaaacagaatctactaaaacaaggcaaaatgccgtgtttatctcgtcaacttgttggcgagattttttctcgaggtcggaggatccacaaaggctagcgtgcactgatcacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcaattgaaaccggtctctaaggtaaatataaaatttttaagtgtataatgtgttaaactactgattctaattgtttctctcttttagattccaacctttggaactgaattctcgaggtgcacaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 9 - DNA, 7490 bp, synthetic construct
  source 1..7490: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcccttgcggccaattcgagctcggtacctccggacgtacgtctagacttttctcttacacagaaggccctgcgataaagtcctggctccctcacatctgtctgtacctgtgttgtgggcccatgtctgcagagacaccctctgcctctgaggagtctcccattgtctcctgtacctggcttctgtcttcccttccgttgctctggggaagatgctctggctgcctccttccacctcctggcagtgtgtgtgggtgttgacgcgtgtgcactgatcacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcaattgaaaccggtctctaaggtaaatataaaatttttaagtgtataatgtgttaaactactgattctaattgtttctctcttttagattccaacctttggaactggtaccgagctcggatccactagtaacggccgccagtgtgctggaattcggctttccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaatcaaagaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttggactagttgtgagaaaccccacccacactgatactggctcctcctctcctcaggccttcagaagtattcatgctgccccgaaccaacaggctttcaccatgaatatacggaagcccctctgttcgaactccgtggttggggcctgcacccttatctctctgactacagcggtcatcctgggtcatctcatgcttcgggagttaatgctgcttccccaaggcatgctggggacgccgcagaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 10 - DNA, 7620 bp, synthetic construct
  source 1..7620: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccaatttcgagctcggtaccctagacagcagggcaacacccaccctggccaccttaccccacctgcctgggtgctgcagtgccagccgcggttgatgtctcagagctgctttgagagccccgtgagtgccgcccctcccgcctccctgctgagcccgctttcttctcccgcaggcctgtaggagctgtccaggccatctccaaccatgggagtgaggcacccgccctgctcccaccggctcctggccgtctgcgccctcgtgtccttggcaaccgctgcactcctggggcacatcctactacatgatttcctgctggttccccgagagctgagtggatcctccccagtcctggaggagacgcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgtctagacatgggttccatggtgtaatggttagcactctggactctgaatccagcgatccgagttcaaatctcggtggaacctggctccctgctggtgagctgggttttagtactctggaaacagaatctactaaaacaaggcaaaatgccgtgtttatctcgtcaacttgttggcgagattttttctcgaggggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 11 - DNA, 7730 bp, synthetic construct
  source 1..7730: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagacatgggttccatggtgtaatggttagcactctggactctgaatccagcgatccgagttcaaatctcggtggaacctcagagctgctttgagagccccgttttagtactctggaaacagaatctactaaaacaaggcaaaatgccgtgtttatctcgtcaacttgttggcgagattttttctcgaggtcggaggatccacaaaggctagcttcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcggacgcgtccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgactagtttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactccaaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 12 - DNA, 7818 bp, synthetic construct
  source 1..7818: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagacatgggttccatggtgtaatggttagcactctggactctgaatccagcgatccgagttcaaatctcggtggaacctcagagctgctttgagagccccgttttagtactctggaaacagaatctactaaaacaaggcaaaatgccgtgtttatctcgtcaacttgttggcgagattttttctcgaggtcggaggatccacaaaggctagcttcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcggacgcgtgtgcactgatcacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcaattgaaaccggtctctaaggtaaatataaaatttttaagtgtataatgtgttaaactactgattctaattgtttctctcttttagattccaacctttggaactgaattctcgaggtgcacactagtttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactccaaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 13 - DNA, 7548 bp, synthetic construct
  source 1..7548: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagacatgggttccatggtgtaatggttagcactctggactctgaatccagcgatccgagttcaaatctcggtggaacctcagagctgctttgagagccccgttttagtactctggaaacagaatctactaaaacaaggcaaaatgccgtgtttatctcgtcaacttgttggcgagattttttctcgaggtcggaggatccacaaaggctagcttcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcggacgcgtctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcactagtttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactccaaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 14 - DNA, 7621 bp, synthetic construct
  source 1..7621: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagacatgggttccatggtgtaatggttagcactctggactctgaatccagcgatccgagttcaaatctcggtggaacctcagagctgctttgagagccccgttttagtactctggaaacagaatctactaaaacaaggcaaaatgccgtgtttatctcgtcaacttgttggcgagattttttctcgaggtcggaggatccacaaaggctagcttcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcggacgcgttgactgactgaaccggtaataaaatatctttattttcattacatctgtgtgttggttttttgtgtgtgaattcctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcactagtttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactccaaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 15 - DNA, 7382 bp, synthetic construct
  source 1..7382: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagacatgggttccatggtgtaatggttagcactctggactctgaatccagcgatccgagttcaaatctcggtggaacctcagagctgctttgagagccccgttttagtactctggaaacagaatctactaaaacaaggcaaaatgccgtgtttatctcgtcaacttgttggcgagattttttctcgaggtcggaggatccacaaaggctagcgtgcactgatcacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcaattgaaaccggtctctaaggtaaatataaaatttttaagtgtataatgtgttaaactactgattctaattgtttctctcttttagattccaacctttggaactgaattctcgaggtgcacaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 16 - DNA, 7490 bp, synthetic construct
  source 1..7490: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcccttgcggccaattcgagctcggtacctccggacgtacgtctagattcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcggacgcgtgtgcactgatcacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcaattgaaaccggtctctaaggtaaatataaaatttttaagtgtataatgtgttaaactactgattctaattgtttctctcttttagattccaacctttggaactggtaccgagctcggatccactagtaacggccgccagtgtgctggaattcggctttccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaatcaaagaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttggactagtttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactccagcatgctggggacgccgcagaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 17 - DNA, 7492 bp, synthetic construct
  source 1..7492: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccaatttcgagctcggtaccgcctgactgtacctagcatactttaaagatggcctctctttcccacccattgtgatctactaaactatcagttcctcttggccctgatccccttgctgctggcccccatgctctgttccagcacacgggtgtgtatgacccaggtgagcccaggtctctgtgttctcatcccactcagggccctggtcaagacgcggggaacacgaccgtttgtgatctcccgctcaaccttctcgggccacggaagatacgctggtcactggactggggatgtgcggagctcttgggagcatcttgcatactctgtgccaggtgagtacaagctgcctgcagggctctggggtcttttctgggggtctttgggctgtgggctcaccactgcagatggtattgtgatagcttggtctagacatgggttccatggtgtaatggttagcactctggactctgaatccagcgatccgagttcaaatctcggtggaacctgatcacaaagggtcgtgttccgttttagtactctggaaacagaatctactaaaacaaggcaaaatgccgtgtttatctcgtcaacttgttggcgagattttttctcgagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 18 - DNA, 7492 bp, synthetic construct
  source 1..7492: mol_type = other DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctgcggccaatttcgagctcggtaccgcctgactgtacctagcatactttaaagatggcctctctttcccacccattgtgatctactaaactatcagttcctcttggccctgatccccttgctgctggcccccatgctctgttccagcacacgggtgtgtatgacccaggtgagcccaggtctctgtgttctcatcccactcagggccctggtcaagacgcggggaacacgaccgtttgtgatctcccgctcaaccttctcgggccacggaagatacgctggtcactggactggggatgtgcggagctcttgggagcatcttgcatactctgtgccaggtgagtacaagctgcctgcagggctctggggtcttttctgggggtctttgggctgtgggctcaccactgcagatggtattgtgatagcttggtctagacatgggttccatggtgtaatggttagcactctggactctgaatccagcgatccgagttcaaatctcggtggaacctaaggttgagcgggagatcacagttttagtactctggaaacagaatctactaaaacaaggcaaaatgccgtgtttatctcgtcaacttgttggcgagattttttctcgagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 19 - DNA, 3255 bp, synthetic construct
  source 1..3255: mol_type = other DNA
atggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaa

SEQ ID NO: 20 - protein, 1085 aa, Staphylococcus aureus
MAPKKKRKVGIHGVPAAKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKGKRPAATKKAGQAKKKK

SEQ ID NO: 21 - protein, 3156 aa, Staphylococcus aureus
AAGAGGAATTACATCCTCGGACTGGACATCGGCATTACCAGCGTCGGCTACGGCATCATCGATTACGAGACAAGGGACGTGATTGACGCTGGCGTCAGGCTGTTCAAGGAGGCCAACGTGGAGAATAATGAGGGCAGGAGAAGCAAGAGGGGCGCCAGGAGGCTGAAGAGGAGGAGAAGGCACAGAATCCAGAGGGTCAAGAAGCTCCTGTTCGACTACAACCTGCTGACCGACCATAGCGAGCTGAGCGGCATTAACCCCTACGAGGCTAGGGTCAAAGGCCTCTCCCAGAAACTGTCCGAGGAGGAGTTTAGCGCTGCTCTCCTGCACCTGGCCAAGAGGAGGGGAGTGCACAATGTGAACGAGGTCGAAGAGGACACCGGCAATGAGCTCAGCACCAAGGAGCAGATTAGCAGAAACAGCAAAGCCCTGGAGGAAAAATATGTCGCCGAGCTACAGCTGGAGAGACTCAAGAAGGATGGCGAGGTCAGAGGCTCCATTAACAGGTTCAAGACCTCCGACTACGTGAAAGAAGCCAAGCAGCTGCTGAAGGTGCAAAAGGCCTATCATCAGCTGGACCAGAGCTTCATTGATACCTATATCGACCTGCTCGAAACCAGGAGAACCTACTATGAGGGCCCTGGAGAAGGAAGCCCCTTCGGATGGAAAGACATTAAGGAGTGGTACGAAATGCTGATGGGACACTGCACCTACTTCCCCGAGGAGCTGAGGAGCGTGAAATATGCCTACAACGCCGATCTCTACAACGCCCTGAACGACCTGAACAATCTCGTGATTACAAGGGATGAGAACGAAAAGCTGGAATATTACGAAAAATTTCAGATCATTGAAAACGTGTTTAAGCAAAAGAAGAAGCCCACCCTGAAGCAGATTGCCAAGGAGATCCTGGTGAACGAAGAGGACATTAAAGGCTACAGAGTGACCTCCACAGGCAAACCCGAATTTACCAATCTGAAGGTGTATCACGATATCAAAGATATCACAGCTAGAAAGGAGATCATCGAGAATGCTGAGCTGCTCGACCAAATCGCCAAGATTCTCACCATCTACCAGAGCAGCGAGGACATCCAGGAAGAACTGACAAACCTGAACTCCGAGCTCACCCAGGAAGAAATCGAGCAGATTTCCAACCTGAAAGGCTACACCGGCACCCACAACCTGTCCCTGAAGGCCATCAACCTGATCCTCGATGAACTCTGGCACACCAATGACAATCAAATCGCTATCTTCAACAGACTGAAGCTCGTGCCCAAGAAAGTGGACCTGAGCCAGCAGAAGGAAATCCCTACAACCCTCGTGGACGATTTCATCCTCAGCCCCGTCGTGAAGAGGAGCTTCATCCAGTCCATCAAGGTGATTAACGCTATCATCAAGAAGTATGGCCTCCCCAACGACATCATCATCGAGCTCGCTAGGGAAAAAAACTCCAAGGATGCTCAGAAAATGATCAACGAGATGCAGAAAAGGAACAGACAAACCAACGAAAGGATTGAGGAGATTATCAGGACCACCGGCAAGGAGAATGCTAAGTATCTGATTGAAAAAATTAAACTCCACGATATGCAGGAGGGCAAGTGTCTCTATAGCCTGGAAGCCATCCCTCTGGAGGATCTCCTGAACAATCCTTTTAACTACGAAGTGGACCACATTATCCCCAGGAGCGTGTCCTTCGATAACTCCTTTAATAATAAGGTCCTGGTCAAACAGGAGGAGAACTCCAAGAAGGGAAACAGGACACCCTTCCAATATCTCTCCAGCAGCGACTCCAAAATAAGCTACGAAACCTTTAAAAAACACATTCTGAACCTGGCCAAGGGCAAAGGCAGGATCAGCAAAACCAAAAAGGAATACCTCCTGGAGGAGAGAGACATCAACAGGTTCTCCGTGCAGAAAGACTTCATCAACAGGAACCTGGTGGACACCAGGTATGCCACCAGGGGCCTGATGAACCTCCTGAGGTCCTATTTTAGAGTCAATAATCTCGACGTGAAAGTCAAATCCATTAATGGCGGCTTTACCAGCTTCCTGAGGAGAAAATGGAAGTTCAAGAAGGAGAGAAACAAAGGCTACAAACACCACGCCGAAGATGCCCTCATCATTGCTAACGCCGACTTCATCTTTAAGGAGTGGAAGAAGCTCGATAAGGCCAAGAAAGTCATGGAGAATCAAATGTTCGAGGAGAAACAAGCCGAATCCATGCCCGAGATTGAAACGGAGCAAGAGTACAAGGAAATATTCATCACCCCTCACCAGATCAAACACATCAAGGACTTCAAAGACTACAAATATAGCCATAGAGTCGATAAGAAGCCCAACAGGGAGCTGATCAACGACACACTCTATTCCACCAGAAAGGATGATAAGGGCAACACCCTGATTGTGAACAACCTGAACGGCCTCTACGATAAGGATAACGACAAGCTGAAGAAGCTGATTAACAAGAGCCCCGAGAAACTGCTCATGTACCACCACGACCCCCAAACATACCAGAAACTGAAGCTGATCATGGAACAGTATGGCGACGAGAAAAACCCCCTGTATAAGTACTATGAAGAAACCGGCAACTACCTCACCAAGTACAGCAAAAAGGATAACGGACCCGTGATCAAAAAGATTAAGTATTACGGCAATAAGCTGAATGCCCACCTCGATATCACCGATGATTACCCCAACTCCAGAAATAAAGTCGTGAAACTGTCCCTGAAACCTTACAGGTTTGACGTGTACCTGGACAATGGCGTGTACAAGTTCGTGACCGTGAAGAACCTCGACGTGATTAAGAAGGAGAATTACTATGAGGTCAACAGCAAATGCTATGAGGAAGCCAAGAAACTGAAGAAGATTTCCAATCAGGCCGAGTTCATCGCCAGCTTTTATAACAACGACCTGATTAAGATCAATGGCGAACTGTATAGGGTGATCGGCGTCAACAACGACCTCCTGAACAGGATTGAGGTCAATATGATCGATATTACCTACAGAGAGTACCTCGAAAACATGAATGACAAGAGGCCCCCCAGGATTATCAAAACCATCGCCAGCAAGACCCAGAGCATTAAGAAATACAGCACCGACATCCTCGGCAACCTGTACGAGGTCAAGTCCAAAAAGCACCCCCAGATTATCAAAAAGGGA

SEQ ID NO: 22 - protein, 1052 aa, Staphylococcus aureus
KRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 23 - DNA, 143 bp, synthetic construct
  source 1..143: mol_type = other DNA
ttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttc

SEQ ID NO: 24 - DNA, 142 bp, synthetic construct
  source 1..142: mol_type = other DNA
aacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 25 - DNA, 143 bp, synthetic construct
  source 1..143: mol_type = other DNA
gaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaa

SEQ ID NO: 26 - protein, 27 aa, synthetic construct
TACCCATACGATGTTCCTGACTATGCG

SEQ ID NO: 27 - protein, 24 aa, synthetic construct
TATCCCTATGACGTCCCGGACTAT

SEQ ID NO: 28 - RNA, 82 nt, synthetic construct
  source 1..82: mol_type = other RNA
gttttagtactctggaaacagaatctactaaaacaaggcaaaatgccgtgtttatctcgtcaacttgttggcgagatttttt

SEQ ID NO: 29 - RNA, 76 nt, synthetic construct
  source 1..76: mol_type = other RNA
gttttagtactctggaaacagaatctactaaaacaaggcaaaatgccgtgtttatctcgtcaacttgttggcgaga

SEQ ID NO: 30 - protein, 623 aa, Homo sapiens
CTAGACAGCAGGGCAACACCCACCCTGGCCACCTTACCCCACCTGCCTGGGTGCTGCAGTGCCAGCCGCGGTTGATGTCTCAGAGCTGCTTTGAGAGCCCCGTGAGTGCCGCCCCTCCCGCCTCCCTGCTGAGCCCGCTTTCTTCTCCCGCAGGCCTGTAGGAGCTGTCCAGGCCATCTCCAACCATGGGAGTGAGGCACCCGCCCTGCTCCCACCGGCTCCTGGCCGTCTGCGCCCTCGTGTCCTTGGCAACCGCTGCACTCCTGGGGCACATCCTACTACATGATTTCCTGCTGGTTCCCCGAGAGCTGAGTGGATCCTCCCCAGTCCTGGAGGAGACGCACCCAGCTCACCAGCAGGGAGCCAGCAGACCAGGGCCCCGGGATGCCCAGGCACACCCCGGCCGTCCCAGAGCAGTGCCCACACAGTGCGACGTCCCCCCCAACAGCCGCTTCGATTGCGCCCCTGACAAGGCCATCACCCAGGAACAGTGCGAGGCCCGCGGCTGTTGCTACATCCCTGCAAAGCAGGGGCTGCAGGGAGCCCAGATGGGGCAGCCCTGGTGCTTCTTCCCACCCAGCTACCCCAGCTACAAGCTGGAGAACCTGAGCTCCTCTGAAATG

SEQ ID NO: 31 - DNA, 400 bp, Homo sapiens
  source 1..400: mol_type = other DNA
ccgcaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggatcctccccagtcctggaggagacgcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagccca

SEQ ID NO: 32 - DNA, 404 bp, Homo sapiens
  source 1..404: mol_type = genomic DNA
gcctgactgtacctagcatactttaaagatggcctctctttcccacccattgtgatctactaaactatcagttcctcttggccctgatccccttgctgctggcccccatgctctgttccagcacacgggtgtgtatgacccaggtgagcccaggtctctgtgttctcatcccactcagggccctggtcaagacgcggggaacacgaccgtttgtgatctcccgctcaaccttctcgggccacggaagatacgctggtcactggactggggatgtgcggagctcttgggagcatcttgcatactctgtgccaggtgagtacaagctgcctgcagggctctggggtcttttctgggggtctttgggctgtgggctcaccactgcagatggtattgtgatagcttgg

SEQ ID NO: 33 - RNA, 21 nt, synthetic construct
  source 1..21: mol_type = other RNA
ggctccctgctggtgagctgg

SEQ ID NO: 34 - RNA, 21 nt, synthetic construct
  source 1..21: mol_type = other RNA
cagagctgctttgagagcccc

SEQ ID NO: 35 - RNA, 21 nt, synthetic construct
  source 1..21: mol_type = other RNA
gatcacaaagggtcgtgttcc

SEQ ID NO: 36 - RNA, 27 nt, synthetic construct
  source 1..27: mol_type = other RNA
gatcacaaagggtcgtgttccccgagt

SEQ ID NO: 37 - RNA, 21 nt, synthetic construct
  source 1..21: mol_type = other RNA
aaggttgagcgggagatcaca

SEQ ID NO: 38 - DNA, 27 bp, synthetic construct
  source 1..27: mol_type = other DNA
aaggttgagcgggagatcacaaagggt

SEQ ID NO: 39 - RNA, 21 nt, synthetic construct
  source 1..21: mol_type = other RNA
ttccacctcctggcagtgtgt

SEQ ID NO: 40 - RNA, 27 nt, synthetic construct
  source 1..27: mol_type = other RNA
ttccacctcctggcagtgtgtgtgggt

SEQ ID NO: 41 - RNA, 21 nt, synthetic construct
  source 1..21: mol_type = other RNA
gcactaccagagctaactcag

SEQ ID NO: 42-43 - skipped (2 entries)

SEQ ID NO: 44 - DNA, 240 bp, synthetic construct
  source 1..240: mol_type = other DNA
tttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 45 - DNA, 249 bp, synthetic construct
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 46 - DNA, 398 bp, synthetic construct
  source 1..398: mol_type = other DNA
tgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagctcgtttagtgaaccgtcagatcgcctggagacaccatccacgctgttttgacctccatagaagagaccgggaccgatccagccta

SEQ ID NO: 47 - DNA, 400 bp, synthetic construct
  source 1..400: mol_type = other DNA
ttgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagctcgtttagtgaaccgtcagatcgcctggagacaccatccacgctgttttgacctccatagaagagaccgggaccgatccagcctaa

SEQ ID NO: 48 - DNA, 408 bp, synthetic construct
  source 1..408: mol_type = other DNA
cggtctatgcatgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagctcgtttagtgaaccgtcagatcgcctggagacaccatccacgctgttttgacctccatagaagagaccgggaccgatccagcct

SEQ ID NO: 49 - DNA, 398 bp, synthetic construct; identical to SEQ ID NO: 46
  source 1..398: mol_type = other DNA

SEQ ID NO: 50 - DNA, 485 bp, Gallus gallus
  source 1..485: mol_type = genomic DNA
ccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcg

SEQ ID NO: 51 - DNA, 691 bp, Gallus gallus
  source 1..691: mol_type = genomic DNA
atatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgaccgtgccttcgccccgtg

SEQ ID NO: 52 - DNA, 1284 bp, Homo sapiens
  source 1..1284: mol_type = genomic DNA
agctcaggaattcaagaccagcctgggcaacatggaaaaaccccatctctacaaaagatagaaaaattagccaggcatggtggcgtgtgcctgtggtcccagctactcaggaggctgaggtgggaggatcacattagcccaggaggttgaggctgcagtgagccgtgattatgccactgcactccagcctgggagacagagtgagaccctgtttcaaaaaaaagagagagaaaatttaaaaaagaaaacaacaccaagggctgtaactttaaggtcattaaatgaattaatcactgcattcaaaaacgattactttctggccctaagagacatgaggccaataccaggaagggggttgatctcccaaaccagaggcagaccctagactctaatacagttaaggaaagaccagcaagatgatagtccccaatacaatagaagttactatattttatttgttgtttttcttttgttttgttttgttttgttttgttttgttttagagactggggtcttgctcgattgcccaggctgtagtgcagcggtgggacaatagctcactgcagactccaactcctgggctcaagcaatcctcctgcctcagcctcctgaatagctgggactacaagggtacaccatcacacacaccaaaacaattttttaaatttttgtgtagaaacgagggtcttgctttgttgcccaggctggtctccaactcctggcttcaagggatcctcccacctcagcctcccaaattgctgggattacaggtgtgagccaccacaaccagccagaactttactaattttaaaattaagaacttaaaacttgaatagctagagcaccaagatttttctttgtccccaaataagtgcagttgcaggcatagaaaatctgacatctttgcaagaatcatcgtggatgtagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttc

SEQ ID NO: 53 - DNA, 303 bp, synthetic construct
  source 1..303: mol_type = other DNA
ctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagc

SEQ ID NO: 54 - DNA, 356 bp, synthetic construct
  source 1..356: mol_type = other DNA
ctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttc

SEQ ID NO: 55 - DNA, 212 bp, synthetic construct
  source 1..212: mol_type = other DNA
gggcagagcgcacatcgcccacagtccccgagaagttggggggaggggtcggcaattgatccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 56 - DNA, 573 bp, synthetic construct
  source 1..573: mol_type = other DNA
gtgcactgatcacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcaattgaaaccggtctctaaggtaaatataaaatttttaagtgtataatgtgttaaactactgattctaattgtttctctcttttagattccaacctttggaactgaattctcgaggtgcac

SEQ ID NO: 57 - DNA, 250 bp, synthetic construct
  source 1..250: mol_type = other DNA
ccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccg

SEQ ID NO: 58 - RNA, 72 nt, synthetic construct
  source 1..72: mol_type = other RNA
ggttccatggtgtaatggttagcactctggactctgaatccagcgatccgagttcaaatctcggtggaacct

SEQ ID NO: 59 - skipped

SEQ ID NO: 60 - DNA, 24 bp, synthetic construct
  source 1..24: mol_type = other DNA
cccaagaagaagcggaaagttggt

SEQ ID NO: 61 - DNA, 48 bp, synthetic construct
  source 1..48: mol_type = other DNA
aagcgacctgccgccacaaagaaggctggacaggctaagaagaagaaa

SEQ ID NO: 62 - DNA, 21 bp, synthetic construct
  source 1..21: mol_type = other DNA
cccaagaagaagcggaaagtt

SEQ ID NO: 63 - DNA, 50 bp, synthetic construct
  source 1..50: mol_type = other DNA
aataaaatatctttattttcattacatctgtgtgttggttttttgtgtgt

SEQ ID NO: 64 - DNA, 194 bp, Homo sapiens
  source 1..194: mol_type = genomic DNA
ctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttgg

SEQ ID NO: 65 - DNA, 212 bp, Homo sapiens
  source 1..212: mol_type = genomic DNA
ttcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcgg

SEQ ID NO: 66 - DNA, 210 bp, Homo sapiens
  source 1..210: mol_type = genomic DNA
cctggggacattctaagcgtgtttgatttgtaacattttagcagactgtgcaagtgctctgcactcccctgctggagcttttctcgcccttccttctggccctctccccagtctagacagcagggcaacacccaccctggccaccttaccccacctgcctgggtgctgcagtgccagccgcggttgatgtctcagagctgctttgagagc

SEQ ID NO: 67 - DNA, 212 bp, Homo sapiens
  source 1..212: mol_type = genomic DNA
ttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactcca

SEQ ID NO: 68 - DNA, 212 bp, Mus musculus
  source 1..212: mol_type = genomic DNA
cttttctcttacacagaaggccctgcgataaagtcctggctccctcacatctgtctgtacctgtgttgtgggcccatgtctgcagagacaccctctgcctctgaggagtctcccattgtctcctgtacctggcttctgtcttcccttccgttgctctggggaagatgctctggctgcctccttccacctcctggcagtgtgtgtgggtgttg

SEQ ID NO: 69 - DNA, 212 bp, Homo sapiens
  source 1..212: mol_type = genomic DNA
tgtgagaaaccccacccacactgatactggctcctcctctcctcaggccttcagaagtattcatgctgccccgaaccaacaggctttcaccatgaatatacggaagcccctctgttcgaactccgtggttggggcctgcacccttatctctctgactacagcggtcatcctgggtcatctcatgcttcgggagttaatgctgcttccccaag

SEQ ID NO: 70 - DNA, 212 bp, Homo sapiens; identical to SEQ ID NO: 67
  source 1..212: mol_type = genomic DNA

SEQ ID NO: 71 - DNA, 210 bp, Homo sapiens
  source 1..210: mol_type = genomic DNA
cccgtcagtgccgcccctcccgcctccctgctgagcccgctttcttctcccgcaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagag

SEQ ID NO: 72 - DNA, 212 bp, Homo sapiens; identical to SEQ ID NO: 65
  source 1..212: mol_type = genomic DNA

SEQ ID NO: 73 - DNA, 2869 bp, Homo sapiens
  source 1..2869: mol_type = genomic DNA
atctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 74 - DNA, 3204 bp, Homo sapiens
  source 1..3204: mol_type = genomic DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaatcaaagaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttgg

SEQ ID NO: 75 - DNA, 2859 bp, Homo sapiens
  source 1..2859: mol_type = genomic DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttag

SEQ ID NO: 76 - DNA, 2856 bp, Homo sapiens
  source 1..2856: mol_type = genomic DNA
atgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 77 - protein, 939 aa, Homo sapiens
ISNHGSEAPALLPPAPGRLRPRVLGNRCTPGAHPTPFPAGSPRAEWLLPSPGGDSPSSPAGSQQTRAPGCPGTPRPSQSSAHTVRRPPQQPLRLRPQGHHPGTVRGPRLLLHPCKAGAAGSPDGAALVLLPTQLPQLQAGEPELLNGLHGHPDPYHPHLLPQGHPDPAAGRDDGDEPPPLHDQRSSQALRGALGDPACPQPGTVPTLQRGVLRGALRGDRAPAAGRPRAAEHDGGAPVLCGPVPSAVHLAALAVYHRPRRAPQSPDAQHQLDQDHPVEPGPCAHARCEPLRVSPFLPGAGGRRVGTRGVPAKQQCHGCGPAAEPCPLEVDRWDPGCLHLPGPRAQERGAAVPGRCGIPVHAAILGPGLPPVPLGLLLHRYHPPGGGEHDQGPLPPGRPVERPGLHGLPEGLHVQQGWLPGLPGHGAGAAPGRPALHDDRGSCHQQLGPCRELQALRRGSAEGGFHHQRDRPAADWEGMARVHCLPRLHQPHSPGLVGGHGGVPPGALRRHVDHERAFQLHQGLGRLPQQAGEPTLRAWGGWGDPPGGHHLCLQPPVSLHTLQPAQPLRPDRSHRLPQGAGEGSGDTPICDLPLDLCWPRPIRRPLDGGRVELLGAARLLRARNPAVPAGGASGRGRRLRLPGQHLRGAVCALDPAGGLLPLHAEPQQPAQSAPGAVQLQRAGPAGHEEGPHPALRTPPPPLHTVPPGPRRGGDRGPAPLPGVPQGLHLDCGPPAPVGGGPAHHPSAPGREGRSDWLLPLGHMVRPADGANRGPWQPPTPTCSSPASHPQRGAVGDAAGPPGHHQRPPPGWVHHPPAGPWPHNHRVPPAAHGPGCGPDQGWGGPRGAVLGRWREPGSAGARGLHTGHLPGQEHDREAGTCDQGSWPAAAEGDCPGRGHGAPAGPLQRCPCLQLHLQPRHQGPGHLCLAVDGRAVSRQLVL

SEQ ID NO: 78 - protein, 1064 aa, Homo sapiens
MGVRHPPCSHRLLAVCALVSLATAALLGHILLHDFLLVPRELSGSSPVLEETHPAHQQGASRPGPRDAQAHPGRPRAVPTQCDVPPNSRFDCAPDKAITQEQCEARGCCYIPAKQGLQGAQMGQPWCFFPPSYPSYKLENLSSSEMGYTATLTRTTPTFFPKDILTLRLDVMMETENRLHFTIKDPANRRYEVPLETPHVHSRAPSPLYSVEFSEEPFGVIVRRQLDGRVLLNTTVAPLFFADQFLQLSTSLPSQYITGLAEHLSPLMLSTSWTRITLWNRDLAPTPGANLYGSHPFYLALEDGGSAHGVFLLNSNAMDVVLQPSPALSWRSTGGILDVYIFLGPEPKSVVQQYLDVVGYPFMPPYWGLGFHLCRWGYSSTAITRQVVENMTRAHFPLDVQWNDLDYMDSRRDFTFNKDGFRDFPAMVQELHQGGRRYMMIVDPAISSSGPAGSYRPYDEGLRRGVFITNETGQPLIGKVWPGSTAFPDFTNPTALAWWEDMVAEFHDQVPFDGMWIDMNEPSNFIRGSEDGCPNNELENPPYVPGVVGGTLQAATICASSHQFLSTHYNLHNLYGLTEAIASHRALVKARGTRPFVISRSTFAGHGRYAGHWTGDVWSSWEQLASSVPEILQFNLLGVPLVGADVCGFLGNTSEELCVRWTQLGAFYPFMRNHNSLLSLPQEPYSFSEPAQQAMRKALTLRYALLPHLYTLFHQAHVAGETVARPLFLEFPKDSSTWTVDHQLLWGEALLITPVLQAGKAEVTGYFPLGTWYDLQTVPIEALGSLPPPPAAPREPAIHSEGQWVTLPAPLDTINVHLRAGYIIPLQGPGLTTTESRQQPMALAVALTKGGEARGELFWDDGESLEVLERGAYTQVIFLARNNTIVNELVRVTSEGAGLQLQKVTVLGVATAPQQVLSNGVPVSNFTYSPDTKVLDICVSLLMGEQFLVSWCPGGVCSLQREAGSPGKQSLCAGSSCVRAWGLLSAIIFNKRGIWNQRILQICGFLPGWHPCDPSPVPLLALEVATPVPTSLVLIKLSCIILSDVSSIILWGGGGWFGARGPSW

SEQ ID NO: 79 - RNA, 7390 nt, Homo sapiens
  source 1..7390: mol_type = genomic RNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccagagctgctttgagagccccgttttagtactctggaaacagaatctactaaaacaaggcaaaatgccgtgtttatctcgtcaacttgttggcgagattttttctcgaggtcggaggatccacaaaggctagccggtctatgcatgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagctcgtttagtgaaccgtcagatcgcctggagacaccatccacgctgttttgacctccatagaagagaccgggaccgatccagcctaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 80 - RNA, 7285 nt, Homo sapiens
  source 1..7285: mol_type = genomic RNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccagagctgctttgagagccccgttttagtactctggaaacagaatctactaaaacaaggcaaaatgccgtgtttatctcgtcaacttgttggcgagattttttctcgaggtcggaggatccacaaaggctagcctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 81 - RNA, 7232 nt, Homo sapiens
  source 1..7232: mol_type = genomic RNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagagagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacccagagctgctttgagagccccgttttagtactctggaaacagaatctactaaaacaaggcaaaatgccgtgtttatctcgtcaacttgttggcgagattttttctcgaggtcggaggatccacaaaggctagcccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgagctacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 82 - DNA, 7627 bp, Homo sapiens
  source 1..7627: mol_type = genomic DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagattcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcggacgcgtatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcgggcgaggcggagaggtgcggcggcagccaatcagagcggcgcgctccgaaagtttccttttatggcgaggcggcggcggcggcggccctataaaaagcgaagcgcgcggcgggcgggagtcgctgcgaccgtgccttcgccccgtggtaccgaattcaggtatcgccaagtctgtaatagctgcaaaggctcagctgtagcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaatcaaagaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttggactagtttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactccagcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 83 - DNA, 7246 bp, Homo sapiens
  source 1..7246: mol_type = genomic DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagattcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcggacgcgtctctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcaagcttggtaccgaattcaggtatcgccaagtctgtaatagctgcaaaggctcagctgtagcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaatcaaagaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttggactagtttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactccagcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 84 - DNA, 7193 bp, Homo sapiens
  source 1..7193: mol_type = genomic DNA
ggggggggggggggggggttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctagatctggtacctccggacgtacgtctagattcaagtgtctacctgccttgctggtcttcctggggacattctaagcgtgtttgatttgtaacattttagcagactgtgcaagtgctctgcactcccctgctggagcttttctcgcccttccttctggccctctccccagtctagacagcagggcaacacccaccctggccaccttaccgcacctgcctggctgctgcagtgccagccgcggacgcgtccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgaagcttggtaccgaattcaggtatcgccaagtctgtaatagctgcaaaggctcagctgtagcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaatcaaagaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttggactagtttgatgtctcagagctgctttgagagccccgtcagtgccgcccctcccgcctccctgctgagcccgctttcttctcccgcaggcctgtaggagctgtccaggccatctccaaccttgggagtgaggcacccgccctgctcccaccggctcctggccgtctgcgccctcgtgtccttggcaaccgctgcactcctggggcacatcctactccagcatgctggggagagatctgaggaacccctagtgatggagttggccactccctctctgcgcgctcgctcgctcactgaggccgcccgggcaaagcccgggcgtcgggcgacctttggtcgcccggcctcagtgagcgagcgagcgcgcagagagggagtggccaacccccccccccccccccctgcagccctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcaatgctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccatatgcggtgtgaaataccgcacagatgcgtaaggagaaaataccgcatcaggaaattgtaaacgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcgcgccattcgccattcaggctacgcaactgttgggaagggcgatcggtgcgggcctcttcgctattacgccaggctgca

SEQ ID NO: 85 - DNA, 22 bp, Mus musculus
  source 1..22: mol_type = genomic DNA
actcccaacttcctccccttgg

SEQ ID NO: 86 - DNA, 22 bp, Mus musculus
  source 1..22: mol_type = genomic DNA
tctgctcctggatgtgcagagg

SEQ ID NO: 87 - DNA, 20 bp, Mus musculus
  source 1..20: mol_type = genomic DNA
atgttcacgtccttcaggcc

SEQ ID NO: 88 - DNA, 18 bp, Mus musculus
  source 1..18: mol_type = genomic DNA
agaggacgcgagctgatg

SEQ ID NO: 89 - DNA, 20 bp, synthetic construct
  source 1..20: mol_type = other DNA
gtgtctacctgccttgctgg

SEQ ID NO: 90 - DNA, 20 bp, synthetic construct
  source 1..20: mol_type = other DNA
agtctccatcatcacgtcca

SEQ ID NO: 91 - DNA, 19 bp, synthetic construct
  source 1..19: mol_type = other DNA
ggaagttgccactccagtg

SEQ ID NO: 92 - DNA, 21 bp, synthetic construct
  source 1..21: mol_type = other DNA
cccattgtctcctgtacctgg

SEQ ID NO: 93 - DNA, 23 bp, synthetic construct
  source 1..23: mol_type = other DNA
gtcctgaagaggactcatgaagg

SEQ ID NO: 94 - DNA, 19 bp, synthetic construct
  source 1..19: mol_type = other DNA
gcagctagtgaaccctgtg

SEQ ID NO: 95 - DNA, 20 bp, synthetic construct
  source 1..20: mol_type = other DNA
acgtcaatagggggcgtact

SEQ ID NO: 96 - DNA, 21 bp, synthetic construct
  source 1..21: mol_type = other DNA
ctcttcactcccaacttcctc

SEQ ID NO: 97 - DNA, 23 bp, synthetic construct
  source 1..23: mol_type = other DNA
gggccatttaccgtaagttatgt

SEQ ID NO: 98 - DNA, 55 bp, synthetic construct
  source 1..55: mol_type = other DNA
ggcagtgtgtgtgggtgttgacgcgtcggccgctctagaccctaaaatgggcaaa

SEQ ID NO: 99 - DNA, 24 bp, synthetic construct
  source 1..24: mol_type = other DNA
agaaaccgcaccgacactgatact

SEQ ID NO: 100 - DNA, 2863 bp, synthetic construct
  source 1..2863: mol_type = other DNA
atctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgt

SEQ ID NO: 101 - DNA, 6233 bp, synthetic construct
  source 1..6233: mol_type = other DNA
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcggccgcttaagctcgagttctgagtcctctaaggtccctcactcccaactcagccccatgtcctgtcaattcccactcagtgtctgatctccttctcctcacctttcccatctcccgtttgacccagcttcctgagctctcctcccattcccctttttggagtcctcctcctctcccagaacccagtaataagtgggctcctccctggcctggacccccgtggtaaccctataaggcgaggcagctgctgtctgaggcagggaggggctggtgtgggaggctaagggcagctgctaagtttagggtggctccttctctcttcttagagacaacaggtggctggggcctcagtgcccagaaaagaaaatgtcttagaggtatcggcatgggcctggaggaggggggacagggcagggggaggcatcttcctcaggacatcgggtcctagaggctcgagtctagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcggtacccgccaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttaggaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttgggaagacacaccatattggtcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatctgcgacgcgaggctggatggccttccccattatgattcttctcgcttccggcggcatcgggatgcccgcgttgcaggccatgctgtccaggcaggtagatgacgaccatcagggacagcttcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 102 - DNA, 5740 bp, synthetic construct
  source 1..5740: mol_type = other DNA
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcggccgcttaagctcgagtctagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcggtacccgccaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttaggaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttgggaagacacaccatattggtcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatctgcgacgcgaggctggatggccttccccattatgattcttctcgcttccggcggcatcgggatgcccgcgttgcaggccatgctgtccaggcaggtagatgacgaccatcagggacagcttcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 103 - DNA, 5793 bp, synthetic construct
  source 1..5793: mol_type = other DNA
cagatctttggccactccctctctgcgcgctcgctcgctcactgaggccgggcgaccaaaggtcgcccgacgcccgggctttgcccgggcggcctcagtgagcgagcgagcgcgcagagagggagtggccaactccatcactaggggttcctggaggggtggagtcgtgacgtgaattacgtcatagggttagggaggtcggccgcttaagctcgagtctagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcagagcaatcaccaccaagcctggaataactgcaagggctctgctgacatcttcggtacccgccaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcatgtccacagccgggcaccgtccccactctacagcgtggagttctccgaggagcccttcggggtgatcgtgcgccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccagtggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcaggggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccagtagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggggaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttaggaattctgcagatctgtggcttctagctgcccgggtggcatccctgtgacccctccccagtgcctctcctggccttggaagttgccactccagtgcccaccagccttgtcctaataaaattaagttgcatcattttgtctgactaggtgtcctctataatattatggggtggaggggggtggtttggagcaaggggcccaagttgggaagacacaccatattggtcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaaagatctgcgacgcgaggctggatggccttccccattatgattcttctcgcttccggcggcatcgggatgcccgcgttgcaggccatgctgtccaggcaggtagatgacgaccatcagggacagcttcagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctg

SEQ ID NO: 104 - DNA, 5950 bp, synthetic construct
  source 1..5950: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctggaggggtggagtcgtgacgtgaattacgtcatagggttagggaggtcggccgctctagactctgtcctgtgtctctggcctggtttcggggaccaggagggcagacccttgcactgccaagaagcatgccaaagttaatcattggccctgctgagtacatggccgatcaggctgtttttgtgtgcctgtttttctattttacgtaaatcaccctgaacatgtttgcatcaacctactggtgatgcacctttgatcaatacattttagacaaacgtggtttttgagtccaaagatcagggctgggttgacctgaatactggatacagggcatataaaacaggggcaaggcacagactcatagcggtaccgagctcggatccactagtaacggccgccagtgtgctggaattcggctttccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgagttgttgggattccaggcatcgagtagataagtagcatggcgggttaatcattaactacaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggacagatctgcgacgcgaggctggatggccttccccattatgattcttctcgcttccggcggcatcgggatgcccgcgttgcaggccatgctgtccaggcaggtagatgacgaccatcagggacagcttcaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctgcaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaacacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgcagatctgggccactccctct

SEQ ID NO: 105 - DNA, 6922 bp, synthetic construct
  source 1..6922: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccaattcgagctcggtacctccggacgtacgtctagacttttctcttacacagaaggccctgcgataaagtcctggctccctcacatctgtctgtacctgtgttgtgggcccatgtctgcagagacaccctctgcctctgaggagtctcccattgtctcctgtacctggcttctgtcttcccttccgttgctctggggaagatgctctggctgcctccttccacctcctggcagtgtgtgtcggtgttgacgcgtcggccgctctagaccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcttaagcttcggctttccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaggaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcaagttgggaagacaacctgagttgttgggattccaggcatcgagtagataagtagcatggcgggttaatcattaactagttgtgagaaaccgcaccgacactgatactggctcctcctctcctcaggccttcagaagtattcatgctgccccgaaccaataggctttcaccatgaatatacggaagcccctctgttcgaactccgtggttggggcctgcacccttatctctctgactacagcggtcatcctgggtcatctcatgcttcgggagttaatgctgcttccccaaggcatgctggggacggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagccaagagtccactattaaagaacgtggactccaacgtcaaagggcgaaaaaccgtctatcagggcgatggcccactacgtgaaccatcacccaaatcaagttttttggggctcaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 106 - RNA, 7067 nt, Mus musculus
  source 1..7067: mol_type = genomic RNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccaattcgagctcggtacctccggacgtacgtctagacatgggttccatggtgtaatggttagcactctggactctgaatccagcgatccgagttcaaatctcggtggaacctaggagccagtatcagtgtggggttttagtactctggaaacagaatctactaaaacaaggcaaaatgccgtgtttatctcgtcaacttgttggcgagattttttctcgaggtgcactgatcacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcaattgaaaccggtctctaaggtaaatataaaatttttaagtgtataatgtgttaaactactgattctaattgtttctctcttttagattccaacctttggaactgaattctcgaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgaactacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggacggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 107 - DNA, 7067 bp, synthetic construct
  source 1..7067: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccaattcgagctcggtacctccggacgtacgtctagacatgggttccatggtgtaatggttagcactctggactctgaatccagcgatccgagttcaaatctcggtggaacctcagagctgctttgagagccccgttttagtactctggaaacagaatctactaaaacaaggcaaaatgccgtgtttatctcgtcaacttgttggcgagattttttctcgaggtgcactgatcacccctaaaatgggcaaacattgcaagcagcaaacagcaaacacacagccctccctgcctgctgaccttggagctggggcagaggtcagagacctctctgggcccatgccacctccaacatccactcgaccccttggaatttcggtggagaggagcagaggttgtcctggcgtggtttaggtagtgtgagaggggaatgactcctttcggtaagtgcagtggaagctgtacactgcccaggcaaagcgtccgggcagcgtaggcgggcgactcagatcccagccagtggacttagcccctgtttgctcctccgataactggggtgaccttggttaatattcaccagcagcctcccccgttgcccctctggatccactgcttaaatacggacgaggacagggccctgtctcctcagcttcaggcaccaccactgacctgggcaattgaaaccggtctctaaggtaaatataaaatttttaagtgtataatgtgttaaactactgattctaattgtttctctcttttagattccaacctttggaactgaattctcgaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgaactacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggacggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 108 - DNA, 7687 bp, synthetic construct
  source 1..7687: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccaattcgagctcggtacctccggacgtacgtctagacttttctcttacacagaaggccctgcgataaagtcctggctccctcacatctgtctgtacctgtgttgtgggcccatgtctgcagagacaccctctgcctctgaggagtctcccattgtctcctgtacctggcttctgtcttcccttccgttgctctggggaagatgctctggctgcctccttccacctcctggcagtgtgtgtcggtgttgacgcgtatatatagttgctctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgcatggtcgaggtgagccccacgttctgcttcactctccccatctcccccccctccccacccccaattttgtatttatttattttttaattattttgtgcagcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcggtaccgagctcggatccactagtaacggccgccagtgtgctggaattcggctttccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggctcctccccagtcctggaggagactcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgctgctacatccctgcaaagcaggggctgcagggagcccagatggggcagccctggtgcttcttcccacccagctaccccagctacaagctggagaacctgagctcctctgaaatgggctacacggccaccctgacccgtaccacccccaccttcttccccaaggacatcctgaccctgcggctggacgtgatgatggagactgagaaccgcctccacttcacgatcaaagatccagctaacaggcgctacgaggtgcccttggagaccccgcgtgtccacagccgggcaccgtccccactctacagcgtggagttctccgaggagcccttcggggtgatcgtgcaccggcagctggacggccgcgtgctgctgaacacgacggtggcgcccctgttctttgcggaccagttccttcagctgtccacctcgctgccctcgcagtatatcacaggcctcgccgagcacctcagtcccctgatgctcagcaccagctggaccaggatcaccctgtggaaccgggaccttgcgcccacgcccggtgcgaacctctacgggtctcaccctttctacctggcgctggaggacggcgggtcggcacacggggtgttcctgctaaacagcaatgccatggatgtggtcctgcagccgagccctgcccttagctggaggtcgacaggtgggatcctggatgtctacatcttcctgggcccagagcccaagagcgtggtgcagcagtacctggacgttgtgggatacccgttcatgccgccatactggggcctgggcttccacctgtgccgctggggctactcctccaccgctatcacccgccaggtggtggagaacatgaccagggcccacttccccctggacgtccaatggaacgacctggactacatggactcccggagggacttcacgttcaacaaggatggcttccgggacttcccggccatggtgcaggagctgcaccagggcggccggcgctacatgatgatcgtggatcctgccatcagcagctcgggccctgccgggagctacaggccctacgacgagggtctgcggaggggggttttcatcaccaacgagaccggccagccgctgattgggaaggtatggcccgggtccactgccttccccgacttcaccaaccccacagccctggcctggtgggaggacatggtggctgagttccatgaccaggtgcccttcgacggcatgtggattgacatgaacgagccttccaacttcatcagaggctctgaggacggctgccccaacaatgagctggagaacccaccctacgtgcctggggtggttggggggaccctccaggcggccaccatctgtgcctccagccaccagtttctctccacacactacaacctgcacaacctctacggcctgaccgaagccatcgcctcccacagggcgctggtgaaggctcgggggacacgcccatttgtgatctcccgctcgacctttgctggccacggccgatacgccggccactggacgggggacgtgtggagctcctgggagcagctcgcctcctccgtgccagaaatcctgcagtttaacctgctgggggtgcctctggtcggggccgacgtctgcggcttcctgggcaacacctcagaggagctgtgtgtgcgctggacccagctgggggccttctaccccttcatgcggaaccacaacagcctgctcagtctgccccaggagccgtacagcttcagcgagccggcccagcaggccatgaggaaggccctcaccctgcgctacgcactcctcccccacctctacacactgttccaccaggcccacgtcgcgggggagaccgtggcccggcccctcttcctggagttccccaaggactctagcacctggactgtggaccaccagctcctgtggggggaggccctgctcatcaccccagtgctccaggccgggaaggccgaagtgactggctacttccccttgggcacatggtacgacctgcagacggtgccaatagaggcccttggcagcctcccacccccacctgcagctccccgtgagccagccatccacagcgaggggcagtgggtgacgctgccggcccccctggacaccatcaacgtccacctccgggctgggtacatcatccccctgcagggccctggcctcacaaccacagagtcccgccagcagcccatggccctggctgtggccctgaccaagggtggagaggcccgaggggagctgttctgggacgatggagagagcctggaagtgctggagcgaggggcctacacacaggtcatcttcctggccaataacacgatcgtgaatgagctggtacgtgtgaccagtgagggagctggcctgcagctgcagaaggtgactgtcctgggcgtggccacggcgccccagcaggtcctctccaacggtgtccctgtctccaacttcacctacagccccgacaccaaggtcctggacatctgtgtctcgctgttgatgggagagcagtttctcgtcagctggtgttagccgggcggagtgtgttagtctctccagagggaggctggttccccagggaagcagagcctgtgtgcgggcagcagctgtgtgcgggcctgggggttgttaagtgcaattatttttaataaaaggggcatttggaaaaaaaaaaaaaggtagcagtcgacagatgaattctgcagatctgtggcttctagctgcccgggtggcatccctgtgacccctccccagtgcctctcctggccctggaagttgccactccagtgcccaccagccttgtcctaataaaattaagttgcatcattttgtctgactaggtgtccttctataatattatggggtggaggggggtggtatggagcaaggggcccaagttggacgcgttgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacgggtgggaggtctatataagcagagctcgtttagtgaaccgtcagatcgcctggagacaccatccacgctgttttgacctccatagaagagaccgggaccgatccagcctaactagttgtgagaaaccgcaccgacactgatactggctcctcctctcctcaggccttcagaagtattcatgctgccccgaaccaataggctttcaccatgaatatacggaagcccctctgttcgaactccgtggttggggcctgcacccttatctctctgactacagcggtcatcctgggtcatctcatgcttcgggagttaatgctgcttccccaaggcatgctggggacggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatcaggcgcc

SEQ ID NO: 109 - DNA, 7336 bp, synthetic construct
  source 1..7336: mol_type = other DNA
cctgcaggcagctgcgcgctcgctcgctcactgaggccgcccgggcaaagcccgggcgtcgggcgacctttggtcgcccggcctcagtgagcgagcgagcgcgcagagagggagtggccaactccatcactaggggttcctgcggccaattcgagctcggtaccgtgagtgccgcccctcccgcctccctgctgagcccgctttcttctcccgcaggcctgtaggagctgtccaggccatctccaaccatgggagtgaggcacccgccctgctcccaccggctcctggccgtctgcgccctcgtgtccttggcaaccgctgcactcctggggcacatcctactccatgatttcctgctggttccccgagagctgagtggatcctccccagtcctggaggagacgcacccagctcaccagcagggagccagcagaccagggccccgggatgcccaggcacaccccggccgtcccagagcagtgcccacacagtgcgacgtcccccccaacagccgcttcgattgcgcccctgacaaggccatcacccaggaacagtgcgaggcccgcggctgttgctacatccctgctctagacatgggttccatggtgtaatggttagcactctggactctgaatccagcgatccgagttcaaatctcggtggaacctaggagccagtatcagtgtggggttttagtactctggaaacagaatctactaaaacaaggcaaaatgccgtgtttatctcgtcaacttgttggcgagattttttctcgaggtcggaggatccacaaaggctagccggtctatgcatgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgtttcggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacaccatccacgctgttttgacctccatagaagagaccgggaccgatccagcctaagcttgccaccatggcacccaagaagaagcggaaagttggtatacacggtgtgcctgcagccaagaggaattacatcctcggactggacatcggcattaccagcgtcggctacggcatcatcgattacgagacaagggacgtgattgacgctggcgtcaggctgttcaaggaggccaacgtggagaataatgagggcaggagaagcaagaggggcgccaggaggctgaagaggaggagaaggcacagaatccagagggtcaagaagctcctgttcgactacaacctgctgaccgaccatagcgagctgagcggcattaacccctacgaggctagggtcaaaggcctctcccagaaactgtccgaggaggagtttagcgctgctctcctgcacctggccaagaggaggggagtgcacaatgtgaacgaggtcgaagaggacaccggcaatgagctcagcaccaaggagcagattagcagaaacagcaaagccctggaggaaaaatatgtcgccgaactacagctggagagactcaagaaggatggcgaggtcagaggctccattaacaggttcaagacctccgactacgtgaaagaagccaagcagctgctgaaggtgcaaaaggcctatcatcagctggaccagagcttcattgatacctatatcgacctgctcgaaaccaggagaacctactatgagggccctggagaaggaagccccttcggatggaaagacattaaggagtggtacgaaatgctgatgggacactgcacctacttccccgaggagctgaggagcgtgaaatatgcctacaacgccgatctctacaacgccctgaacgacctgaacaatctcgtgattacaagggatgagaacgaaaagctggaatattacgaaaaatttcagatcattgaaaacgtgtttaagcaaaagaagaagcccaccctgaagcagattgccaaggagatcctggtgaacgaagaggacattaaaggctacagagtgacctccacaggcaaacccgaatttaccaatctgaaggtgtatcacgatatcaaagatatcacagctagaaaggagatcatcgagaatgctgagctgctcgaccaaatcgccaagattctcaccatctaccagagcagcgaggacatccaggaagaactgacaaacctgaactccgagctcacccaggaagaaatcgagcagatttccaacctgaaaggctacaccggcacccacaacctgtccctgaaggccatcaacctgatcctcgatgaactctggcacaccaatgacaatcaaatcgctatcttcaacagactgaagctcgtgcccaagaaagtggacctgagccagcagaaggaaatccctacaaccctcgtggacgatttcatcctcagccccgtcgtgaagaggagcttcatccagtccatcaaggtgattaacgctatcatcaagaagtatggcctccccaacgacatcatcatcgagctcgctagggaaaaaaactccaaggatgctcagaaaatgatcaacgagatgcagaaaaggaacagacaaaccaacgaaaggattgaggagattatcaggaccaccggcaaggagaatgctaagtatctgattgaaaaaattaaactccacgatatgcaggagggcaagtgtctctatagcctggaagccatccctctggaggatctcctgaacaatccttttaactacgaagtggaccacattatccccaggagcgtgtccttcgataactcctttaataataaggtcctggtcaaacaggaggagaactccaagaagggaaacaggacacccttccaatatctctccagcagcgactccaaaataagctacgaaacctttaaaaaacacattctgaacctggccaagggcaaaggcaggatcagcaaaaccaaaaaggaatacctcctggaggagagagacatcaacaggttctccgtgcagaaagacttcatcaacaggaacctggtggacaccaggtatgccaccaggggcctgatgaacctcctgaggtcctattttagagtcaataatctcgacgtgaaagtcaaatccattaatggcggctttaccagcttcctgaggagaaaatggaagttcaagaaggagagaaacaaaggctacaaacaccacgccgaagatgccctcatcattgctaacgccgacttcatctttaaggagtggaagaagctcgataaggccaagaaagtcatggagaatcaaatgttcgaggagaaacaagccgaatccatgcccgagattgaaacggagcaagagtacaaggaaatattcatcacccctcaccagatcaaacacatcaaggacttcaaagactacaaatatagccatagagtcgataagaagcccaacagggagctgatcaacgacacactctattccaccagaaaggatgataagggcaacaccctgattgtgaacaacctgaacggcctctacgataaggataacgacaagctgaagaagctgattaacaagagccccgagaaactgctcatgtaccaccacgacccccaaacataccagaaactgaagctgatcatggaacagtatggcgacgagaaaaaccccctgtataagtactatgaagaaaccggcaactacctcaccaagtacagcaaaaaggataacggacccgtgatcaaaaagattaagtattacggcaataagctgaatgcccacctcgatatcaccgatgattaccccaactccagaaataaagtcgtgaaactgtccctgaaaccttacaggtttgacgtgtacctggacaatggcgtgtacaagttcgtgaccgtgaagaacctcgacgtgattaagaaggagaattactatgaggtcaacagcaaatgctatgaggaagccaagaaactgaagaagatttccaatcaggccgagttcatcgccagcttttataacaacgacctgattaagatcaatggcgaactgtatagggtgatcggcgtcaacaacgacctcctgaacaggattgaggtcaatatgatcgatattacctacagagagtacctcgaaaacatgaatgacaagaggccccccaggattatcaaaaccatcgccagcaagacccagagcattaagaaatacagcaccgacatcctcggcaacctgtacgaggtcaagtccaaaaagcacccccagattatcaaaaagggaaagcgacctgccgccacaaagaaggctggacaggctaagaagaagaaaggatcctacccatacgatgttcctgactatgcgggctatccctatgacgtcccggactatgcatgagtcgacaataaaatatctttattttcattacatctgtgtgttggttttttgtgtgtgcatgctggggacggccgcaggaacccctagtgatggagttggccactccctctctgcgcgctcgctcgctcactgaggccgggcgaccaaaggtcgcccgacgcccgggcggcctcagtgagcgagcgagcgcgcagctgcctgcaggacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccataaaattgtaaacgttaatattttgttaaaattcgcgttaaatttttgttaaatcagctcattttttaaccaatagaccgaaatcggcaaaatcccttataaatcaaaagaatagcccgagatagagttgagtgttgttccagtttggaacaagagtccactattaaagaacgtggactccaacgtcaaagggcgaaaaaccgtctatcagggcgatggcccactacgtgaaccatcacccaaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctaaggcgctggcaagtgtagcggtcacgctgcgcgtaaccaccacacccgccgcgcttaatgcgccgctacagggcgcgtactatggttgctttgacgtatgcggtgtgaaataccgcacagatgcgtaaggagaaaataccgcatc